(19) United States
(12) Patent Application Publication
(10) Pub. No.: US 2019/0113522 A1
(43) Pub. Date: Apr. 18, 2019

(54) ARRAY-BASED PEPTIDE LIBRARIES FOR THERAPEUTIC ANTIBODY CHARACTERIZATION

(71) Applicant: HealthTell Inc., San Ramon, CA (US)

(72) Inventors: Matthew GREVING, Phoenix, AZ (US); David SMITH, Scottsdale, AZ (US); Gaurav SAINI, Chandler, AZ (US)

(21) Appl. No.: 16/090,549

(22) PCT Filed: Mar. 31, 2017
(86) PCT No.: PCT/US2017/025546

§ 371 Date: Oct. 1, 2018

Related U.S. Application Data
(60) Provisional application No. 62/317,353, filed on Apr. 1, 2016.
(60) Provisional application No. 62/472,504, filed on Mar. 16, 2017.

(51) Int. Cl. G01N 33/68; B01J 19/00; C40B 50/18; C40B 40/10

(52) U.S. Cl. CPC G01N 33/6854; B01J 19/0046; C40B 50/18; C40B 40/10; G01N 2333/71; B01J 2219/00675; B01J 2219/00623; B01J 2219/00725; B01J 2219/00711; C40B 50/02

(57) ABSTRACT

Provided herein are methods, chemical library and simulation system for performing in situ patterned chemistry. Methods, systems and assays comprising the use of the synthesized chemical libraries, which increase explored protein space in a knowledge-based manner, are also provided for characterizing antibody-target interactions including: identifying target proteins of antibodies, characterizing antibody-binding regions in target proteins, identifying linear and structural epitopes in target proteins, and determining the propensity of antibody binding to target proteins.

### CROSS-REFERENCE TO RELATED APPLICATIONS

This application claims the benefit of U.S. Provisional Application No. 62/317,353, filed Apr. 1, 2016, and the benefit of U.S. Provisional Application No. 62/472,504, filed Mar. 16, 2017, both of which are incorporated herein by reference in their entirety.

### BACKGROUND OF THE INVENTION

Cancer is the second most common cause of death in the United States, with more than 1,600 cancer related deaths per day, nearly 600,000 per year, in the U.S. Approximately 1.65 million new cases of cancer were diagnosed in 2015 and cancer incidence is increasing due to demographic and lifestyle factors. Sensitive and effective methods for detection and treatment of cancer is needed.

### SUMMARY OF THE INVENTION

Cancer deaths have been on the decline with recent improvements in diagnostics and therapeutics, and cancer is moving towards a chronic disease with continual monitoring and follow-on treatment. While the fraction of cancer patient deaths are declining, the financial burden of cancer treatment is increasing rapidly due to the high cost of breakthrough therapeutics and prolonged chronic care that includes cancer relapse and additional therapeutic treatments. This rapid increase in the cost of cancer treatment is on an unsustainable trajectory and at the current rate, out-of-pocket cost for the patient will be 100% median household income by the year 2028. As a result of rising costs, particularly the cost of cancer immunotherapeutics and antibody therapeutics, patients are required to make difficult choices between treatment and financial stability.

Immunotherapy and antibody-based treatment of cancer have been two major therapeutic breakthroughs in extending patient survival. Immunotherapy activates and utilizes the patient's immune system to kill cancer cells, whereas antibody-based therapeutics target specific pathways that inhibit or kill cancer cells. Each of these approaches rely heavily or exclusively on the discovery and development of highly target-specific antibodies or biologics and more recently, multi target-specific antibodies or biologics with multivalent binding. Even with the significant advancement in patient survival offered by immunotherapy and antibody-based treatment, specific major challenges remain. First, immunotherapeutic and antibody-based treatments have limited patient groups that respond favorably due to the high occurrence of major off-target side-effects. For example, two of the most prescribed antibody therapeutics Humira and Remicade are only effective in 25% of the patient population. Second, high discovery and development costs are entry barriers that limit the number of immunotherapy and antibody-based R&D programs and competitors in the market. Both the high occurrence of off-target effects in a significant fraction of patients, and the high R&D costs, result in a very high price for immunotherapy and antibody-based treatments that in many cases are prohibitively expensive for a patient.

One of the major threats to current pharmaceutical R&D is decreasing productivity due to escalating R&D costs. Alleviating this decrease in productivity will require innovations that reduce costs, increase the number of candidate molecules in-progress and reduce R&D cycle-time. To reduce high R&D costs and off-target risks associated with immunotherapy and antibody-based treatments, innovative platforms are needed to enable comprehensive screening and characterization of therapeutic antibody leads from early in the discovery process to late-stage pre-clinical development. In addition, new lower-cost and higher-throughput antibody characterization platforms will allow for more candidates to enter the discovery pipeline and enable additional companies to enter into immunotherapeutic discovery programs, which will increase innovation, competition and market potential.

Immunotherapy is a breakthrough in cancer treatment and one of the fastest growing pharmaceutical market areas. Antibody library screening and on-/off-target binding characterization are essential activities in immunotherapy development. Currently a large gap exists between the capability to routinely screen large antibody libraries against therapeutic targets and the limited ability to characterize on-/off-target binding of the screen-selected therapeutic antibody candidates. This gap is widening with the advent of multi-specific therapeutic antibodies and biologics where the number of candidates is much larger than mono-specific antibodies. A major limitation in therapeutic antibody on-/off-target binding characterization is the miniscule fraction of epitope interactions that can be profiled relative to the total possible epitopes (e.g. 10-mer peptide epitope implies 10 trillion possible sequences). Current antibody characterization platforms, including microarrays, surface plasmon resonance (SPR) and interferometry, have practical limitations of 10,000-50,000 epitope interaction measurements. Such limited therapeutic antibody binding profiles increase the risk of undetected off-target effects.

New platforms are disclosed herein to dramatically increase the number of detected therapeutic antibody interactions, which may reduce this risk of undetected off-target effects. The technologies are based on merged peptide synthesis chemistry with semiconductor manufacturing processes by utilizing mask-based photolithography to pattern, in situ, libraries containing more than 40 million peptides (potential epitopes) on an eight-inch wafer. This wafer is diced into 13 microscope-slide dimensioned chips for downstream analysis. With such a peptide library chips described herein, antibody binding profile assays can be scaled to more than 10 million antibody-target interactions per day at a fraction of the cost of current antibody characterization platforms. Antibody epitope point-variant analysis demonstrates the applicability of the peptide chips to antibody characterization.

In one aspect, disclosed herein is a method of in situ synthesizing a chemical library on a substrate, the chemical library comprising a plurality of molecules, the method comprising: (a) receiving a biological sequence and a number of synthesis steps; (b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask; (c) assigning at least one monomer to each patterned mask; and (d) coupling the monomers onto the features to form molecules; wherein (c) and (d) assembles one said synthesis step and the synthesis step is repeated. In some embodiments, the number of synthesis steps is larger than 50%, 60%, 70%, 80%, 90%, 100%, 110%, 120%, 130%, 140%, 150%, 160%, 170%, 180%, 190%, or 200% of a length of the biological sequence. In some embodiments, the input biological sequence comprises a disease-related epitope. In some embodiments, the input biological sequence comprises a peptide sequence. In some embodiments, the input biological sequence comprises an epitope sequence. In some embodiments, the input biological sequence comprises a random sequence. In some embodiments, the method comprises deriving an ordered list of monomers from the input biological sequence. In additional embodiments, a size of the ordered list is the number of the synthesis steps. In some embodiments, the ordered list of monomers comprises the input biological sequence. In some embodiments, the ordered list of monomers comprises the input biological sequence in a reversed order. In some embodiments, molecules are peptides or nucleic acids. In some embodiments, the ordered list of monomers comprises a sequence of amino acids. In some embodiments, the ordered list of monomers comprises a sequence of nucleotides. In some embodiments, a number of the plurality of the patterned masks is less than 10, 15, 20, 25, 30, 35, 40, 45, 50, 60, 70, 80, 90, or 100. In some embodiments, a number of the plurality of the patterned masks is the number of the synthesis steps. In some embodiments, about 20% to about 50% of the activated designation features in each sequential patterned mask overlaps with the activated designation features of an immediately preceding patterned mask. In some embodiments, about 30% to about 45% of the activated designation features in each sequential patterned mask overlaps with the activated designation features of an immediately preceding patterned mask. In some embodiments, the synthesis step is based on photolithography. In some embodiments, a feature on the substrate is about 0.5 micron to about 200 microns in diameter and a center-to-center distance of about 1 micron to about 300 microns on center. In some embodiments, at least 40% of the molecules in the library are distinct. In some embodiments, at least 50% of the molecules in the library are distinct. In some embodiments, at least 60% of the molecules in the library are distinct. In some embodiments, at least 70% of the molecules in the library are distinct. In some embodiments, at least 80% of molecules in the library are distinct. In some embodiments, at least 90% of molecules in the library are distinct. In some embodiments, at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers. In some embodiments, the library comprises a median monomer length equal to a length of the biological sequence. In some embodiments, the library comprises a median monomer length longer than 40%, 50%, 60%, 70%, 80%, or 90% of a length of the biological sequence. In some embodiments, the library comprises a median monomer length shorter than 60%, 70%, 80%, 90%, 100%, 110%, 120%, 130%, 140%, 150%, 160%, 170%, 180%, 190%, or 200% of a length of the biological sequence. In some embodiments, the substrate is selected from the group consisting of arrays, wafers, slides and beads. In some embodiments, the synthesized chemical library comprises peptides, nucleotides or a combination thereof. In some embodiments, the peptides are about 5 to about 25 amino acids in length. In some embodiments, the amino acids C, I, and M, and optionally Q and E, are not included in the amino acids available for peptide synthesis. In some embodiments, the chemical library is synthesized with a surface spacer capable of cyclizing under oxidizing conditions. In some embodiments, the surface spacer is Cys-Gly-Pro-Gly-Xaan-Gly-Pro-Gly-Cys or Cys-(PEG3)-Xaan-(PEG3)-Cys. In some embodiments, the chemical library is synthesized with a surface spacer capable of cyclizing with an ester linkage. In some embodiments, the ester linkage is a homobifunctional di-NHS ester linkage. In some embodiments, the surface spacer is Lys-(PEG3)-Xaan-(PEG3)-Lysine. In some embodiments, the substrate is coated with a hydrophilic monolayer. In some embodiments, the hydrophilic monolayer comprises polyethylene glycol (PEG), polyvinyl alcohol, carboxymethyl dextran, and combinations thereof. In some embodiments, the hydrophilic monolayer is homogeneous.

In another aspect, disclosed herein is an in situ synthesized chemical library, wherein the synthesis uses patterned steps to construct the library on a substrate, the chemical library comprising a plurality of molecules, comprising: (a) receiving a biological sequence and a number of synthesis steps; (b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask; (c) assigning at least one monomer to each patterned mask; and (d) coupling the monomers onto the features to form molecules; wherein (c) and (d) assembles one said synthesis step and the synthesis step is repeated. In some embodiments, the number of synthesis steps is larger than 50%, 60%, 70%, 80%, 90%, 100%, 110%, 120%, 130%, 140%, 150%, 160%, 170%, 180%, 190%, or 200% of a length of the biological sequence. In some embodiments, the input biological sequence comprises a disease-related epitope. In some embodiments, the input biological sequence comprises a peptide sequence. In some embodiments, the input biological sequence comprises an epitope sequence. In some embodiments, the input biological sequence comprises a random sequence. In some embodiments, the method comprises deriving an ordered list of monomers from the input biological sequence. In additional embodiments, a size of the ordered list is the number of the synthesis steps. In some embodiments, the ordered list of monomers comprises the input biological sequence. In some embodiments, the ordered list of monomers comprises the input biological sequence in a reversed order. In some embodiments, molecules are peptides or nucleic acids. In some embodiments, the ordered list of monomers comprises a sequence of amino acids. In some embodiments, the ordered list of monomers comprises a sequence of nucleotides. In some embodiments, a number of the plurality of the patterned masks is less than 10, 15, 20, 25, 30, 35, 40, 45, 50, 60, 70, 80, 90, or 100. In some embodiments, a number of the plurality of the patterned masks is the number of the synthesis steps. In some embodiments, about 20% to about 50% of the activated designation features in each sequential patterned mask overlaps with the activated designation features of an immediately preceding patterned mask. In some embodiments, about 30% to about 45% of the activated designation features in each sequential patterned mask overlaps with the activated designation features of an immediately preceding patterned mask. In some embodiments, the synthesis step is based on photolithography. In some embodiments, a feature on the substrate is about 0.5 micron to about 200 microns in diameter and a center-to-center distance of about 1 micron to about 300 microns on center. In some embodiments, at least 40% of the molecules in the library are distinct. In some embodiments, at least 50% of the molecules in the library are distinct. In some embodiments, at least 60% of the molecules in the library are distinct. In some embodiments, at least 70% of the molecules in the library are distinct. In some embodiments, at least 80% of molecules in the library are distinct. In some embodiments, at least 90% of molecules in the library are distinct. In some embodiments, at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers. In some embodiments, the library comprises a median monomer length equal to a length of the biological sequence. In some embodiments, the library comprises a median monomer length longer than 40%, 50%, 60%, 70%, 80%, or 90% of a length of the biological sequence. In some embodiments, the library comprises a median monomer length shorter than 60%, 70%, 80%, 90%, 100%, 110%, 120%, 130%, 140%, 150%, 160%, 170%, 180%, 190%, or 200% of a length of the biological sequence. In some embodiments, the substrate is selected from the group consisting of arrays, wafers, slides and beads. In some embodiments, the synthesized chemical library comprises peptides, nucleotides or a combination thereof. In some embodiments, the peptides are about 5 to about 25 amino acids in length. In some embodiments, the amino acids C, I, and M, and optionally Q and E, are not included in the amino acids available for peptide synthesis. In some embodiments, the chemical library is synthesized with a surface spacer capable of cyclizing under oxidizing conditions. In some embodiments, the surface spacer is Cys-Gly-Pro-Gly-Xaan-Gly-Pro-Gly-Cys or Cys-(PEG3)-Xaan-(PEG3)-Cys. In some embodiments, the chemical library is synthesized with a surface spacer capable of cyclizing with an ester linkage. In some embodiments, the ester linkage is a homobifunctional di-NHS ester linkage. In some embodiments, the surface spacer is Lys-(PEG3)-Xaan-(PEG3)-Lysine. In some embodiments, the substrate is coated with a hydrophilic monolayer. In some embodiments, the hydrophilic monolayer comprises polyethylene glycol (PEG), polyvinyl alcohol, carboxymethyl dextran, and combinations thereof. In some embodiments, the hydrophilic monolayer is homogeneous.

In another aspect, disclosed herein is a computing system for simulating in situ synthesis of a chemical library on a substrate, the chemical library comprising a plurality of molecules, comprising: (a) a processor and a memory; (b) a computer program including instructions executable by the processor, the computer program comprising: (1) a receiving module configured to receive a biological sequence and a number of synthesis steps; (2) a simulation module configured to: (i) determine a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask; (ii) assign at least one monomer to each patterned mask; and (iii) couple the monomers onto the features to form molecules; wherein (i), (ii) and (iii) assembles one said synthesis step and the synthesis step is repeated. In some embodiments, the number of synthesis steps is larger than 50%, 60%, 70%, 80%, 90%, 100%, 110%, 120%, 130%, 140%, 150%, 160%, 170%, 180%, 190%, or 200% of a length of the biological sequence. In some embodiments, the input biological sequence comprises a disease-related epitope. In some embodiments, the input biological sequence comprises a peptide sequence. In some embodiments, the input biological sequence comprises an epitope sequence. In some embodiments, the input biological sequence comprises a random sequence. In some embodiments, the simulation module comprises deriving an ordered list of monomers from the input biological sequence. In additional embodiments, a size of the ordered list is the number of the synthesis steps. In some embodiments, the ordered list of monomers comprises the input biological sequence. In some embodiments, the ordered list of monomers comprises the input biological sequence in a reversed order. In some embodiments, molecules are peptides or nucleic acids. In some embodiments, the ordered list of monomers comprises a sequence of amino acids. In some embodiments, the ordered list of monomers comprises a sequence of nucleotides. In some embodiments, a number of the plurality of the patterned masks is less than 10, 15, 20, 25, 30, 35, 40, 45, 50, 60, 70, 80, 90, or 100. In some embodiments, a number of the plurality of the patterned masks is the number of the synthesis steps. In some embodiments, about 20% to about 50% of the activated designation features in each sequential patterned mask overlaps with the activated designation features of an immediately preceding patterned mask. In some embodiments, about 30% to about 45% of the activated designation features in each sequential patterned mask overlaps with the activated designation features of an immediately preceding patterned mask. In some embodiments, the synthesis step is based on photolithography. In some embodiments, a feature on the substrate is about 0.5 micron to about 200 microns in diameter and a center-to-center distance of about 1 micron to about 300 microns on center. In some embodiments, at least 40% of the molecules in the library are distinct. In some embodiments, at least 50% of the molecules in the library are distinct. In some embodiments, at least 60% of the molecules in the library are distinct. In some embodiments, at least 70% of the molecules in the library are distinct. In some embodiments, at least 80% of molecules in the library are distinct. In some embodiments, at least 90% of molecules in the library are distinct. In some embodiments, at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length. In some embodiments, the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers. In some embodiments, the library comprises a median monomer length equal to a length of the biological sequence. In some embodiments, the library comprises a median monomer length longer than 40%, 50%, 60%, 70%, 80%, or 90% of a length of the biological sequence. In some embodiments, the library comprises a median monomer length shorter than 60%, 70%, 80%, 90%, 100%, 110%, 120%, 130%, 140%, 150%, 160%, 170%, 180%, 190%, or 200% of a length of the biological sequence. In some embodiments, the substrate is selected from the group consisting of arrays, wafers, slides and beads. In some embodiments, the synthesized chemical library comprises peptides, nucleotides or a combination thereof. In some embodiments, the peptides are about 5 to about 25 amino acids in length. In some embodiments, the amino acids C, I, and M, and optionally Q and E, are not included in the amino acids available for peptide synthesis. In some embodiments, the chemical library is synthesized with a surface spacer capable of cyclizing under oxidizing conditions. In some embodiments, the surface spacer is Cys-Gly-Pro-Gly-Xaan-Gly-Pro-Gly-Cys or Cys-(PEG3)-Xaan-(PEG3)-Cys. In some embodiments, the chemical library is synthesized with a surface spacer capable of cyclizing with an ester linkage. In some embodiments, the ester linkage is a homobifunctional di-NHS ester linkage. In some embodiments, the surface spacer is Lys-(PEG3)-Xaan-(PEG3)-Lysine. In some embodiments, the substrate is coated with a hydrophilic monolayer. In some embodiments, the hydrophilic monolayer comprises polyethylene glycol (PEG), polyvinyl alcohol, carboxymethyl dextran, and combinations thereof. In some embodiments, the hydrophilic monolayer is homogeneous.

Also included are methods and assays for characterizing antibody binding against at least one protein target, the method comprising: (a) contacting a peptide array with said antibody at one or more concentrations in the presence and absence of a plurality of competitor peptides at one or more concentrations to identify one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides; (b) aligning the individual peptides to said at least one protein target, wherein the alignments between the individual peptides of step (a) and at least one protein target are assigned alignment scores; and (c) characterizing binding of the antibody against the at least one protein target using the alignment scores of step (b).

Also disclosed herein are methods and assays for identifying an antibody epitope in a target protein, the method comprising: (a) contacting a peptide array with said antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (b) aligning the individual peptides to said at least one protein target, wherein the alignments between the individual peptides of step (a) and at least one protein target are assigned alignment scores; and (c) determining conserved amino acids in the individual peptides of step (a) to identify a conserved binding peptide motif and aligning the individual motifs to said at least one target protein in order to identify at least one antibody epitope of the target protein.

Disclosed herein are methods and assays for characterizing antibody binding regions in a target protein, the method comprising: (a) contacting a first peptide array with said antibody in the presence and absence of a plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides; (b) creating a second peptide array using an input peptide sequence chosen from at least one of the individual peptides in step (a), a conserved motif derived from an alignment of the individuals peptides in step (a) or an aligned motif derived from an alignment of the individual peptides in step (a), the second peptide array synthesized by: i. determining a number of synthesis steps; ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask; iii. assigning at least one monomer to each patterned mask; and iv. coupling the monomers onto the features, wherein (c) and (d) assembles one said synthesis step and said synthesis step is repeated to form the peptide array; (c) contacting said second peptide array with said antibody to identify a second set of peptides; (d) contacting said second peptide array with said antibody in the presence of a plurality of competitor peptides, and identifying a second set of individual peptides from step (c) that exhibit a binding signal within a second predetermined threshold of the binding signal in step (c); and (e) aligning said second set of individual peptides to said target protein and identifying regions in the target protein which align to the second set of individual peptides identified, thereby characterizing antibody binding regions in the target protein.

Also included herein are methods and assays for identifying a target protein of an antibody, the method comprising: (a) contacting a first peptide array with said antibody at one or more concentrations in the presence and absence of a plurality of competitor peptides at one or more concentrations to obtain one or more input amino acid sequences, wherein the identified input amino acid sequences exhibit a binding signal in the presence of the plurality of competitor peptides within a first predetermined threshold of the binding signal in the absence of the plurality of competitor peptides; (b) obtaining one or more secondary peptide array(s) using one or more input amino acid sequences chosen from at least one of the individual peptides in step (a), a conserved motif derived from an alignment of the individuals peptides in step (a) or an aligned motif derived from an alignment of the individual peptides in step (a), the one or more secondary peptide arrays synthesized by: (i) determining a number of synthesis steps; (ii) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask; (iii) assigning at least one monomer to each patterned mask; and (iv) coupling the monomers onto the features, wherein (iii) and (iv) assembles one said synthesis step and said synthesis step is repeated to form the peptide array; (c) contacting each of said secondary peptide array(s) with said antibody in the presence and absence of the plurality of competitor peptides to obtain a set of peptide sequences, wherein the identified set of peptide sequences exhibit a binding signal measured in the presence of the plurality of competitor peptides within a second predetermined threshold of the binding signal measured in the absence of the plurality of competitor peptides; (d) aligning said set of peptide sequences with each other to obtain at least one predictive binding motif; and (e) aligning said predictive binding motif as a search criteria against a protein database, thereby identifying target proteins of the antibody based on the protein database search results score.

Also included herein are methods for determining the propensity of antibody binding to at least one protein target, the method comprising: (a) contacting a peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (b) aligning the individual peptides of step (a) to a first protein target, wherein the alignments between the individual peptides of step (a) and the first protein target are assigned alignment scores; (c) repeating the alignment of individual peptides of step (a) with at least one additional protein target(s), wherein the alignments between the individual peptides of step (a) and the additional protein targets are assigned alignment scores; and (d) comparing the alignment scores from steps (b) and (c) to obtain a relative propensity of the antibody to bind to said protein targets.

Disclosed herein are methods and assays for determining the propensity of antibody binding to at least one protein target, the method comprising: (a) contacting a first peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (b) aligning the one or more individual peptides of step (a) to obtain at least one predictive target motif; (c) aligning the at least one predictive target motif to a first protein target, wherein the alignments between the individual peptides of step (a) and the first protein target are assigned alignment scores; (d) repeating the alignment of at least one predictive target motif of step (b) with at least one additional protein target(s), wherein the alignments between the at least one predictive target motif of step (b) and the additional protein target(s) are assigned alignment scores; and (e) comparing the alignment scores from steps (c) and (d) to obtain a relative propensity of the antibody to bind to said protein targets.

Also disclosed herein are kits and systems for characterizing antibody binding against at least one protein target, the kits and systems comprising: (a) providing a peptide array, (b) providing a plurality of competitor peptides, (c) providing instructions for a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides; (d) providing instructions for the user to align the individual peptides to said at least one protein target, wherein the alignments between the individual peptides of step (c) and at least one protein target are assigned alignment scores; and (e) providing instructions for the user to characterize binding of the antibody against the at least one protein target using the alignment scores of step (d).

Additionally, kits and systems are disclosed herein for identifying an antibody epitope in a target protein, the kits and systems comprising: (a) providing a peptide array; (b) providing a plurality of competitor peptides; (c) providing instructions for a user to contact the peptide array with said antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (d) providing instructions for the user to align the individual peptides to said at least one protein target, wherein the alignments between the individual peptides of step (c) and at least one protein target are assigned alignment scores; and (e) providing instructions for the user to determine conserved amino acids in the individual peptides of step (c) to identify a conserved binding peptide motif and aligning the individual motifs to said at least one target protein in order to identify at least one antibody epitope of the target protein.

Also disclosed herein are kits and systems for identifying an antibody epitope in a target protein, the kits and systems comprising: (a) providing a peptide array; (b) providing a plurality of competitor peptides; (c) providing instructions for a user to contact the peptide array with said antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (d) providing instructions for the user to align the individual peptides to said at least one protein target, wherein the alignments between the individual peptides of step (c) and at least one protein target are assigned alignment scores; and (e) providing instructions for the user to determine conserved amino acids in the individual peptides of step (c) to identify a conserved binding peptide motif and aligning the individual motifs to said at least one target protein in order to identify at least one antibody epitope of the target protein.

Further disclosed herein are kits and systems for characterizing antibody binding regions in a target protein, the kits and systems comprising: (a) providing a first peptide array; (b) providing a plurality of competitor peptides; (c) providing instructions for a user to contact a first peptide array with an antibody in the presence and absence of the plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides; (d) providing instructions for a user to create a second peptide array using an input peptide sequence chosen from at least one of the individual peptides in step (c), a conserved motif derived from an alignment of the individuals peptides in step (c) or an aligned motif derived from an alignment of the individual peptides in step (c), the second peptide array synthesized by: (i) determining a number of synthesis steps; (ii) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask; (iii) assigning at least one monomer to each patterned mask; and (iv) coupling the monomers onto the features, wherein (ii) and (iii) assembles one said synthesis step and said synthesis step is repeated to form the peptide array; (e) providing instructions for the user to contact the second peptide array with the antibody to identify a second set of peptides; (f) providing instructions for the user to contact the second peptide array with said antibody in the presence of the plurality of competitor peptides, and identifying a second set of individual peptides from step (e) that exhibit a binding signal within a second predetermined threshold of the binding signal in step (e); and (g) providing instructions for a user to align said second set of individual peptides to said target protein and identifying regions in the target protein which align to the second set of individual peptides identified, thereby characterizing antibody binding regions in the target protein.

Also disclosed herein are kits and systems for determining the propensity of antibody binding to at least one protein target, the kit comprising: (a) providing a peptide array; (b) providing a plurality of competitor peptides; (c) providing instructions to a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (d) providing instructions to the user to align the individual peptides of step (c) to a first protein target, wherein the alignments between the individual peptides of step (c) and the first protein target are assigned alignment scores; (e) providing instructions to the user to repeat the alignment of individual peptides of step (c) with at least one additional protein target(s), wherein the alignments between the individual peptides of step (c) and the additional protein targets are assigned alignment scores; and (f) providing instructions to the user to compare the alignment scores from steps (c) and (d) to obtain a relative propensity of the antibody to bind to said protein targets.

Disclosed herein are kits and systems for determining the propensity of antibody binding to at least one protein target, the kits and systems comprising: (a) providing a first peptide array; (b) providing a plurality of competitor peptides; (c) providing instructions for a user to contact the first peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides; (d) providing instructions for the user to align the one or more individual peptides of step (c) to obtain at least one predictive target motif; (e) providing instructions for the user to align the at least one predictive target motif to a first protein target, wherein the alignments between the individual peptides of step (c) and the first protein target are assigned alignment scores; (f) providing instructions for the user to repeat the alignment of at least one predictive target motif of step (e) with at least one additional protein target(s), wherein the alignments between the at least one predictive target motif of step (e) and the additional protein target(s) are assigned alignment scores; and (g) providing instructions for the user to compare the alignment scores from steps (c) and (d) to obtain a relative propensity of the antibody to bind to said protein targets.

In some of the methods, assays, kits and systems disclosed herein, the predetermined threshold is a binding signal in the presence of competitor peptides within at least 20-fold of the binding signal in the absence of competitor peptides. In some disclosures, the predetermined threshold is a binding signal in the presence of competitor peptides within at least 18-fold, within at least 16-fold, within at least 14-fold, within at least 12-fold, within at least 10-fold, within at least 9-fold, within at least 8-fold, within at least 7-fold, within at least 6-fold, within at least 5-fold, within at least 4-fold, within at least 3-fold, within at least 2-fold, within at least 1-fold, within at least 0.5-fold or within at least 0.2-fold of the binding signal in the absence of competitor peptides. In other methods, assays, kits and systems disclosed herein, the predetermined threshold is a binding signal in the presence of competitor peptides of at least 5% of the binding signal as compared in the absence of competitor. In other methods, assays, kits and systems disclosed herein, the predetermined threshold is a binding signal in the presence of competitor peptides of at least 10%, at least 15%, at least 20%, at least 25%, at least 30%, at least 35%, at least 40%, at least 45%, at least 50%, at least 55%, at least 60%, at least 65%, at least 70%, at least 80%, at least 85%, at least 90%, at least 95%, at least 100% of the binding signal as compared in the absence of competitor. In some embodiments, the competitor peptides comprise a biological sample. In other embodiments, the biological sample is serum. In yet other embodiments, the competitor peptides are derived from the target protein. In still other embodiments, the competitor peptides are at least 50% similar to the target protein. In some embodiments, the competitor peptides are at least 55% similar, at least 60% similar, at least 65% similar, at least 70% similar, at least 75% similar, at least 80% similar, at least 85% similar, at least 90% similar, at least 95% similar, at least 97% similar or at least 100% similar to the target protein. In some embodiments, the competitor peptides are derived from a known epitope of the antibody. In some embodiments, the competitor peptides are at least 50% similar to the known epitope of the antibody. In other embodiments, the competitor peptides are the competitor peptides are at least 55% similar, at least 60% similar, at least 65% similar, at least 70% similar, at least 75% similar, at least 80% similar, at least 85% similar, at least 90% similar, at least 95% similar, at least 97% similar or at least 100% similar to the known epitope of the antibody. In still other embodiments, the competitor peptides comprise a biological sample and a peptide derived from the target protein as disclosed herein.

In some embodiments, the peptide array comprises at least 1000 unique peptides. In other embodiments, the peptide array comprises at least 10,000 unique peptides. In still other embodiments, the peptide array comprises at least 100,000 unique peptides. In yet other embodiments, the peptide array comprises at least 1,000,000 unique peptides. In other embodiments, the peptide array comprises at least 5000, at least 10,000, at least 50,000, at least 100,000, at least 250,000, at least 500,000, at least 750,000, at least 1,000,000, at least 2,000,000, at least 3,000,000 or more unique peptides. In still other embodiments, the peptide array is in situ synthesized. In yet other embodiments, the peptide array is synthesized by: a. receiving an input amino acid sequence; b. determining a number of synthesis steps; c. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask; d. assigning at least one monomer to each patterned mask; and e. coupling the monomers onto the features, wherein (c) and (d) assembles one said synthesis step and said synthesis step is repeated to form the peptide array.

In still other embodiments, the binding signal is measured as an intensity of the signal in the absence and presence of the competitor peptides at one or more concentrations. In some embodiments, an apparent Kd is determined in the presence and absence of the competitor peptides at one or more concentrations. In some embodiments, at least one additional antibody is contacted with the peptide array, and the alignment scores obtained with each antibody are ranked to determine the propensity of each antibody to bind to the protein target. The methods, assays, kits and systems disclosed herein also further comprise determining a metric score for each antibody, wherein each antibody is assigned a single binding profile metric derived from the combination of the alignment scores from step (b) as disclosed herein and the signal of the individual peptides of step (a) with more than one aligned position from step (b). The methods, assays, kits and systems disclose herein also further comprise determining a metric score for each antibody, wherein each antibody is assigned a single specificity profile metric derived from the combination of the alignment scores from step (b) as disclosed herein, the number of peptides with more than one aligned position from step (b) and the signal of the individual peptides of step (a) with more than one aligned position from step (b).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings in the following.

FIG. 1 illustrates a photolithographic process for building a peptide array.

FIG. 2 shows an example image taken from a microscopy.

FIG. 3 shows a mass spectrum acquired directly from a single array feature on a peptide library array.

FIG. 4 shows alanine scanning of the p53Ab1 monoclonal antibody epitope (RHSVV).

FIG. 5 shows a graphical representation of a mask algorithm.

FIG. 6 illustrates a sequence of masks.

FIG. 7 shows a graphical representation of ordered synthesis steps.

FIG. 8 shows an example distribution of peptide lengths from in silico simulated peptide library synthesis.

FIG. 9 shows an example distribution of sequence lengths in simulated library generated using the mask and synthesis algorithm disclosed herein

FIG. 10 illustrates a process for obtaining epitope sequences from a focused library of array peptides from epitope motifs obtained from a focused library of array peptides from epitope motifs obtained from a diverse library.

FIG. 11 illustrates the process of identifying an exemplary linear epitope in a target protein by obtaining a HER2 enriched epitope motif from individual peptides, including significant peptides, bound by an anti-HER2 mAb in a diverse library (A), and using the motif to provide a focused library of array peptides that comprises individual peptides, including significant peptides, bound by the anti-HER2 mAb (B), from which a full epitope sequence of HER2 is identified (C). The amino acids most frequently identified in the HER2-aligned individual peptides, including significant peptides, by ClustalW alignment are shown as a WebLogo [Crooks G E et al., (2004) Genome Res 14: 1188-1190]. The corresponding HER2 sequence (UniProt ID=#P04626) is displayed along the x axis. Amino acids at any one position are shown vertically and the proportional occurrence in the aligned significant library peptides is depicted by the height of the one-letter code.

FIG. 12 illustrates an exemplary scoring of alignments of trimers present in individual peptides, including significant peptides, of a diverse library that were bound by anti-HER2 mAbs.

FIG. 13 shows an exemplary mapping of a reduced set of amino acids identified in peptides from a diverse library to a full set of amino acids of a focused peptide array library.

FIG. 14A shows an alignment of individual peptides, including significant peptides, identified from a diverse library in a dose-response assay of anti-HER2 mAb Thermo MA5-13675 (clone 3B5).

FIG. 14B shows array peptide sequences (left column) and the corresponding alignments (right column) of individual peptides, including significant peptides, identified from a focused library in a dose-response assay of anti-HER2 mAb (Thermo MA5-13675 (clone 3B5)).

FIG. 14C shows amino acids most frequently identified in the HER2-aligned peptides shown as a WebLogo.

FIG. 14D shows the corresponding identity of the known immunogen and predicted epitope sequence.

FIG. 15A shows an alignment of individual peptides, including significant peptides, identified from a diverse library in a dose-response assay of anti-HER2 mAb (Santa Cruz SC-33684 (clone 3B5).

FIG. 15B shows array peptide sequences (left column) and the corresponding alignments (right column) of individual peptides, including significant peptides, identified from a focused library in a dose-response assay of anti-HER2 mAb (Santa Cruz SC-33684 (clone 3B5)).

FIG. 15C shows the amino acids most frequently identified in the HER2-aligned peptides shown as a WebLogo.

FIG. 15D shows the corresponding identity of the known immunogen and predicted epitope sequence.

FIG. 16A shows an alignment of individual peptides, including significant peptides, identified from a diverse library in a dose-response assay of anti-HER2 mAb (Cell Signaling 2165 (clone 29D8)).

FIG. 16B shows the amino acids most frequently identified in the HER2-aligned peptides shown as a WebLogo of individual peptides, including significant peptides, identified from a focused library in a dose-response assay of anti-HER2 mAb (Cell Signaling 2165 (clone 29D8)).

FIG. 16C shows the amino acids most frequently identified in the HER2-aligned peptides shows as a WebLogo.

FIG. 16D shows the corresponding identity of the known immunogen and predicted epitope sequence.

FIG. 17A illustrates the linear components and the structural epitope of HER2 identified from alignment of individual peptides, including significant peptides.

FIG. 17B shows the alignment of individual peptides, including significant peptides, in a focused library bound by anti-HER2 mAbs.

FIG. 18 shows the sequences of the linear components of the structural epitope of HER2 identified as illustrated in FIG. 17 and the interaction of anti-HER2 mAb Trastuzumab Fab (Herceptin) crystal structure with the linear components.

FIG. 19 shows the results of a BLAST alignment of the 10 top individual peptides, including significant peptides, (A), and the median 10 peptide (B) identified from a focused library in a dose-response assay of anti-HER2 mAb (Cell Signaling 2165 (clone 29D8)) (B).

FIG. 20 shows the results of a BLAST alignment of the 10 top individual peptides, including significant peptides, (A), and the median 10 peptide (B) identified from a focused library in a dose-response assay of anti-HER2 mAb anti-HER2 mAb (Thermo MA5-13675 (clone 3B5) (B).

FIG. 21 shows the results of a BLAST alignment of the 10 top individual peptides, including significant peptides, (A), and the median 10 peptide (B) identified from a focused library in a dose-response assay of anti-HER2 mAb anti-HER2 mAb (Santa Cruz SC-33684 (clone 3B5)) (B).

FIG. 22 shows the propensity of an anti-HER2 mAb ((Cell Signalling (#2165)) for HER2 and EGFR.

### DETAILED DESCRIPTION OF THE INVENTION

Immunotherapy is a type of cancer treatment that utilizes the body's immune system to seek out and treat cancer. A highly active area of immunotherapy development is the ability to engineer antibodies or biologics that target cell surface receptors such as T-cell inhibitory receptors (e.g. anti-CTLA-4, anti-PD-1) hijacked by cancer cells (i.e. checkpoint therapy). Some approaches are based on engineering multi-specific antibodies that target multiple receptors such as the BiTE antibody architecture that brings T-cells and cancer cells together with a single bi-specific molecule. These multi-specific architectures introduce additional challenges such as more numerous lead candidates that need to be characterized and the potential for increased off-target binding. While antibodies have proven to be a flexible and therapeutically relevant platform in pharmaceutical research and development (R&D), significant limitations exist in the ability to comprehensively characterize on- and off-target binding activity of candidate antibodies from early-discovery through late-stage development.

Synthesized peptide libraries are commonly used for antibody binding characterization, but this is expensive and limited to a small sample of sequence space (i.e. epitope mapping/binning). Antibody characterization with synthesized peptide libraries is currently performed with relatively low-throughput methods such as surface plasmon resonance and interferometry that are limited to measurement of less than 10,000 antibody-peptide interactions (e.g. 20 antibodies vs. 500 peptides). Protein and peptide microarrays can be used to characterize greater than 10,000 antibody-peptide interactions, but protein and robotically printed peptide arrays have been cost-prohibitive and in situ synthesized peptide arrays have suffered from lack of scalability, reproducibility and production quality. Phage or yeast peptide display libraries are also used to identify antibody-peptide interactions, but these iterative selection methods only provide data on the highest affinity interactions and many moderate affinity, clinically relevant antibody-target interactions are left undetected. These limitations in antibody-target characterization ultimately increase development costs due to lead candidate failures commonly the result of undetected off-target binding effects.

The technologies disclosed herein will enable reliable, high-throughput, low-cost and comprehensive binding characterization of therapeutic antibody and biologic lead candidates. For example, benefits of the technologies include: 1) Increasing the number of lead candidates that can be characterized, 2) Improving the success rates of lead candidates, and 3) Lowering immunotherapy development costs. The technologies disclosed herein include a highly scalable array-based peptide library platform based on in situ peptide synthesis with processes and equipment developed for semiconductor manufacturing. The methods and assays disclosed herein also provide the ability to identify antibody binding regions, including epitopes and putative epitopes, as well as protein targets to antibodies, allowing elucidation of possible off-target proteins that could play a role in, for example, adverse or non-target interactions.

### Array Platform

Disclosed herein are methods and process that provide for array platforms that allow for increased diversity and fidelity of chemical library synthesis, The array platforms comprises a plurality of individual features on the surface of the array. Each feature typically comprises a plurality of individual molecules synthesized in situ on the surface of the array, wherein the molecules are identical within a feature, but the sequence or identity of the molecules differ between features. The array molecules include, but are not limited to nucleic acids (including DNA, RNA, nucleosides, nucleotides, structure analogs or combinations thereof), peptides, peptide-mimetics, and combinations thereof and the like, wherein the array molecules may comprise natural or non-natural monomers within the molecules. Such array molecules include the synthesis of large synthetic peptide arrays. In some embodiments, a molecule in an array is a mimotope, a molecule that mimics the structure of an epitope and is able to bind an epitope-elicited antibody. In some embodiments, a molecule in the array is a paratope or a paratope mimetic, comprising a site in the variable region of an antibody (or T cell receptor) that binds to an epitope of an antigen. In some embodiments, an array of the invention is a peptide array comprising random, semi-random or diverse peptide sequences. In some embodiments, the diverse peptide sequences may be derived from a proteome library, for example, from a specific organism (see, e.g., Mycobacterium tuberculosis (Mtb) proteome library (Schubert et al., Cell Host Microbe (2013) 13(5):602-12), or organelle (see, e.g., Mitochondrial (Mtd) proteome library (Calvo and Mootha, Annu. Rev. Genomics (2010) 11:25-44), and the like.

In yet other embodiments, the diverse peptide sequences may be derived from a set of all known combinations of amino acids, for example at least 100% of all possible tetramers, at least 90% of all possible tetramers, at least 85% of all possible tetramers, at least 80% of all possible tetramers, at least 75% of all possible tetramers, at least 70% of all possible tetramers, at least 65% of all possible tetramers, at least 60% of all possible tetramers, at least 55% of all possible tetramers, at least 50% of all possible tetramers, at least 45% of all possible tetramers, at least 40% of all possible tetramers, at least 35% of all possible tetramers, at least 30% of all possible tetramers, or at least 25% of all possible tetramers. In still other embodiments, the diverse peptide sequences may be derived from a set of all possible pentamers, for example, at least 100% of all possible pentamers, at least 95% of all possible pentamers, at least 90% of all possible pentamers, at least 85% of all possible pentamers, at least 80% of all possible pentamers, at least 75% of all possible pentamers, at least 70% of all possible pentamers, at least 65% of all possible pentamers, at least 60% of all possible pentamers, at least 55% of all possible pentamers, at least 50% of all possible pentamers, at least 45% of all possible pentamers, at least 40% of all possible pentamers, at least 35% of all possible pentamers, at least 30% of all possible pentamers or at least 25% of all possible pentamers. In yet other embodiments, the diverse peptide sequences of an array may be derived from a set of amino acid combinations, for example from 25%-100% of all possible hexamers, from 25%-100% of all possible septamers, from 25%-100% of all possible octamers, from 25%-100% of all possible nonamers or from 25%-100% of all possible decamers, or combinations thereof. Representation of the diverse peptide sequences is only limited by the size of the array. Accordingly, large arrays, for example, at least 1 million, at least 2 million, at least 3 million, at least 4 million, at least 5 million, at least 6 million, at least 7 million, at least 8 million, at least 9 million, at least 10 million or more peptides can be used with the methods, systems and assays disclosed herein. Alternatively or additionally, multiple substantially non-overlapping peptide libraries/arrays may be synthesized to cover the sequence space needed for resolution of the peptide sequences or motif(s) recognized by the biological sample or antibody.

In some embodiments, the individual peptides on the array are of variable and/or different lengths. In some embodiments, the peptides are between about 6-20 amino acids in length, or between about 7-18 amino acids in length, or between about 8-15 amino acids in length, or between about 9-14 amino acids in length. In other embodiments, the peptides are at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids in length. In still other embodiments, the peptides are not more than 15 amino acids, not more than 14 amino acids, not more than 13 amino acids, not more than 12 amino acids, not more than 11 amino acids, not more than 10 amino acids, not more than 9 amino acids or not more than 8 amino acids in length. In still other embodiments, the peptides on the array have an average length of about 6 amino acids, about 7 amino acids, about 8 amino acids, about 9 amino acids, about 10 amino acids, about 11 amino acids, about 12 amino acids, about 13 amino acids, about 14 amino acids, or about 15 amino acids.

In yet other embodiments, the amino acid building blocks for the peptides on the array comprises all natural amino acids. In other embodiments, the amino acid building blocks for the peptides on the array are comprised of non-natural or synthetic amino acids. In yet other embodiments, only 19 amino acids are used as the building blocks for synthesizing the peptides on the array. In still other embodiments, only 18 amino acids, only 17 amino acids, only 16 amino acids, only 15 amino acids or only 14 amino acids are used as the building blocks for synthesizing the peptides on the array. In some embodiments, cysteine is omitted during peptide synthesis. In other embodiments, methionine is omitted during peptide synthesis. In still other embodiments, isoleucine is omitted during peptide synthesis. In yet other embodiments, threonine is omitted during peptide synthesis. In still other embodiments, cysteine, methionine, isoleucine and/or threonine, including all combinations thereof, are omitted during peptide synthesis.

In some embodiments, an array of the invention is a peptide array comprising a focused or limited set of peptide sequences, all derived from an input amino acid or peptide sequence, or an input amino acid or peptide motif. One or more peptide arrays may be used with the methods, systems and assays disclosed herein, including a diverse or semi-random peptide array and/or a focused or limited set of peptide sequences. For example, the methods, systems and assays disclosed herein may utilize both a diverse set of peptides and a focused or limited set of peptides are chosen. The peptide arrays may be used either in parallel or sequentially with a biological sample as disclosed herein. For example, a diverse peptide array may be used initially, and at least one motif (either sequence or structure-based) or sequence is obtained for a monoclonal antibody, for example, with an unknown binding profile. The identified motif or sequence may be then used as the input sequence for the creation of at least one focused or limited set of peptide sequences, and assays performed as described herein. Using the methods, systems and arrays described herein, multiple focused or limited set of peptide arrays may be used to characterize antibody binding for the unknown monoclonal antibody.

Nearly all therapeutic antibody screens incorporate some level of epitope mapping and epitope binning on a select number of leads and these data drive decisions on which leads move forward into the development pipeline. Epitope mapping studies commonly utilize systematic overlapping sequences of peptides to determine the amino acids responsible for the antibody-target interaction. Epitope binning studies map the epitopes of several lead antibodies and then bin the antibodies by their binding affinity/kinetics towards identified epitopes. Epitope binning studies are a key decision dataset to identify lead antibodies with different epitope reactivity and potentially different modes-of-action and off-target effects. Typically epitope binning and mapping characterizations are done using synthesized libraries of targeted peptide sequences related to known epitope(s), which limits analyses to a few thousand targeted interactions (e.g. 10 lead antibodies vs. 100 peptides) due to limited analysis throughput and the high cost of purified synthetic peptide libraries. Characterization of such a small number of antibody-target interactions allows many off-target and/or low-affinity interactions to go undetected which increases failure rates of candidates late in the development pipeline.

A common weakness of all current epitope mapping/binning platforms is severely limited antibody-epitope interaction analysis throughput relative to the total number of possible interactions. This analytical throughput limitation forces antibody discovery scientists to reduce the number of leads selected for further development. As a result, the reduced number of leads increases the risk of late-stage antibody therapeutic candidate failure. This ultimately increases the cost of those candidates that do succeed and in turn subsidize the R&D costs of failed candidates. Risks associated with limited analytical throughput are increasing with the advent of multi-specific antibody screens that require selection of more numerous lead antibodies to identify candidates with particular multi-specificity relevant to the target disease and minimal off-target effects.

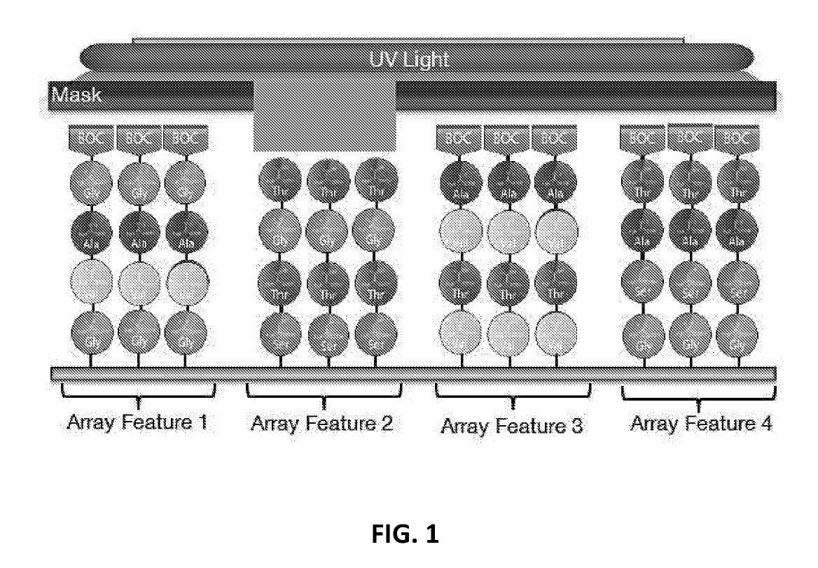

The technologies disclosed herein include a photolithographic array synthesis platform that merges semiconductor manufacturing processes and combinatorial chemical synthesis to produce array-based libraries on silicon wafers. FIG. 1 shows a profile view of a photolithographic process; a platform comprises a substrate 101 to grow peptides synthesis. Applying a mask 102 followed by UV light 103 can control peptide synthesis. Further, by sequentially applying another mask with UV light exposure, various array features can be established. By utilizing the tremendous advancements in photolithographic feature patterning, the array synthesis platform is highly-scalable and capable of producing combinatorial chemical libraries with 40 million features on an 8-inch wafer. Photolithographic array synthesis is performed using semiconductor wafer production equipment in a class 10,000 cleanroom to achieve high reproducibility. When the wafer is diced into standard microscope slide dimensions, each slide contains more than 3 million distinct chemical entities.

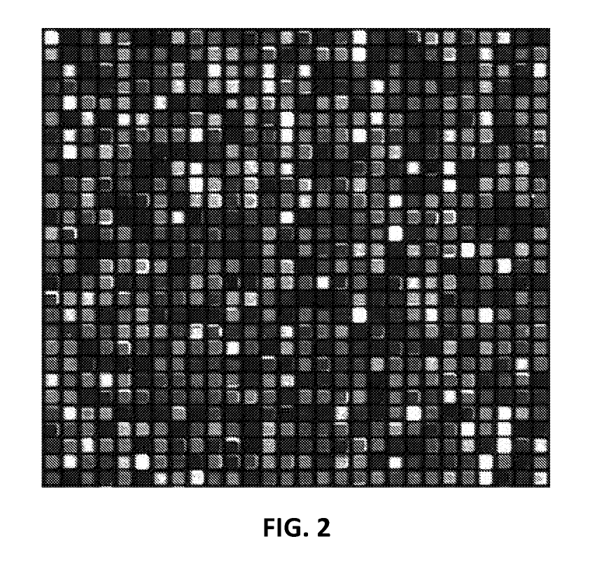

In some embodiments, arrays with chemical libraries produced by the technologies disclosed herein are used for immune-based diagnostic assays, for example called immunosignature assays. Using a patient's antibody repertoire from a drop of blood bound to the arrays, a fluorescence binding profile image of the bound array provides sufficient information to classify disease vs. healthy. FIG. 2 shows an example image taken from a microscopy. The image comprises a fluorescence image of the IgG antibody repertoire bound to the array. Each square feature is 14 μm² and pattered at a density of more than 3 million distinct peptides on a microscope slide.

In some embodiments, immunosignature assays are being developed for clinical application to diagnose/monitor autoimmune diseases and to assess response to autoimmune treatments. Exemplary embodiments of immunosignature assays is described in detail in US Pre-Grant Publication No. 2012/0190574, entitled “Compound Arrays for Sample Profiling” and US Pre-Grant Publication No. 2014/0087963, entitled “Immunosignaturing: A Path to Early Diagnosis and Health Monitoring”, both of which are incorporated by reference herein for such disclosure. The arrays developed herein incorporate analytical measurement capability within each synthesized array using orthogonal analytical methods including ellipsometry, mass spectrometry and fluorescence. These measurements enable longitudinal qualitative and quantitative assessment of array synthesis performance.

One of the major deficiencies of in situ synthesized peptide arrays has been the inability to directly measure purity of the synthesized peptide features. In some embodiments, the technologies include qualitative in situ mass spectrometry of synthesized peptides directly from the silicon wafer. Mass spectrometry is performed by incorporating a gas-phase cleavable linker between the silicon surface and the synthesized peptides so that cleavage of the peptide is done without diffusion from the array feature. Following peptide cleavage, Matrix-Assisted Laser Desorption Ionization (MALDI) mass spectrometry is performed directly on the silicon surface by applying a thin aerosol matrix layer and subsequently focusing the MALDI laser on individual peptide features to acquire a mass spectrum for each synthesized peptide.

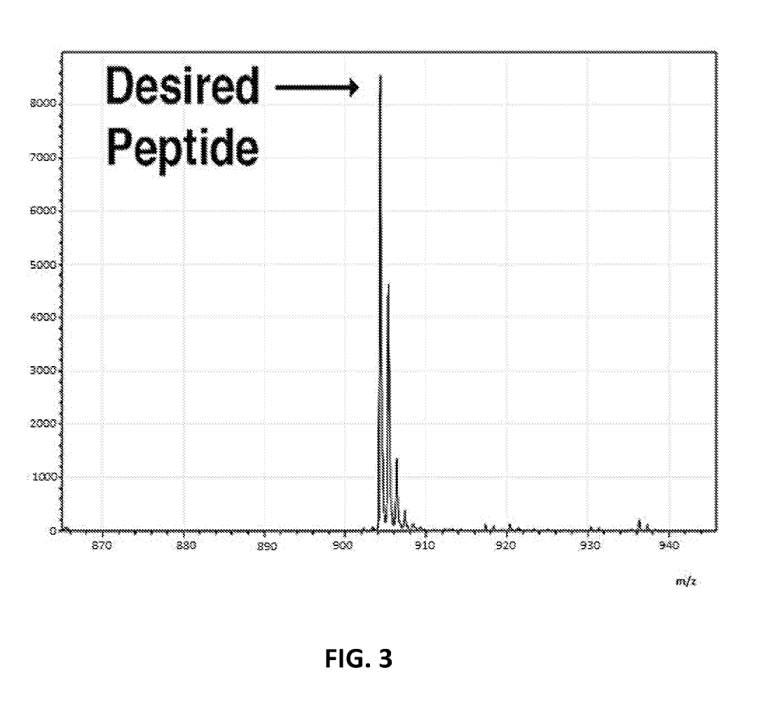

FIG. 3 shows a mass spectrum acquired directly from a single array feature on a peptide library array. Qualitative in situ MALDI mass spectrum from a peptide array feature produced using the photolithographic synthesis approach are also included in the methods and devices described herein. Other analyses known to those of skill in the art may also be used to quantify and/or qualify the fidelity of the in situ synthesis process disclosed herein.

### Binding of Antibodies to Peptide Arrays

In various embodiments, the methods, systems and technologies disclosed herein provide peptide array platforms for detecting binding events, including antibody to peptide binding events, occurring on the peptide arrays. In some embodiments, the peptide arrays are high density peptide arrays. In some embodiments, the arrays comprise individual peptides within a feature on the array spaced less than 0.5 nm, less than 1 nm, less than 2 nm, less than 3 nm, less than 4 nm, less than 5 nm, less than 6 nm, less than 7 nm, less than 8 nm, less than 9 nm, less than 10 nm apart, less than 11 nm apart, less than 12 nm apart, less than 13 nm apart, less than 14 nm part or less than 15 nm apart.

Biological samples are added and allowed to incubate with the peptide arrays. Biological samples include blood, dried blood, serum, plasma, saliva, tears, tear duct fluid, check swab, biopsy, tissue, skin, hair, cerebrospinal fluid sample, feces, or urine sample. In some embodiments, a subject can, for example, use a “fingerstick”, or “fingerprick” to draw a small quantity of blood and add it to a surface, such as a filter paper or other absorbent source, or in a vial or container and optionally dried. A biological sample provided by a subject can be concentrated or dilute. In yet other embodiments, a biological sample is a purified antibody preparation, including a monoclonal antibody, a polyclonal antibody, an antibody fragment, single chain antibodies, chimeric antibodies, humanized antibodies, an antibody drug conjugate or the like. In yet other embodiments, a biological sample is a cell culture or other growth medium used to propagate recombinant antibodies in cell hosts.

In some embodiments, no more than about about 0.5 nl to about 50 μl of biological sample is required for analysis by a method or system as disclosed herein. In yet other embodiments, about 0.5 nl to 25 μl about 5 nl to 10 μl about 5 nl to 5 μl about 10 nl to 5 μl, about 10 nl to 2.5 μl about 100 nl to 2.5 μl or about 100 nl to 1 μl of biological sample is required for analysis. In some embodiments, a subject can provide a solid biological sample, from for example, a biopsy or a tissue. In some embodiments, about 1 mg, about 5 mgs, about 10 mgs, about 15 mgs, about 20 mgs, about 25 mgs, about 30 mgs, about 35 mgs, about 40 mgs, about 45 mgs, about 50 mgs, about 55 mgs, about 60 mgs, about 65 mgs, about 7 mgs, about 75 mgs, about 80 mgs, about 85 mgs, about 90 mgs, about 95 mgs, or about 100 mgs of biological sample are required for analysis by a method or system as disclosed herein.

In some embodiments, biological samples from a subject are too concentrated and require a dilution prior to being contacted with an array of the invention. A plurality of dilutions can be applied to a biological sample prior to contacting the sample with an array of the invention. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

### Detection of Binding Events

Binding interactions between components of a sample and a peptide array can be detected in a variety of formats. In some formats, components of the samples are labeled. The label can be a radioisotype or dye among others. The label can be supplied either by administering the label to a patient before obtaining a sample or by linking the label to the sample or selective component(s) thereof.

Binding interactions can also be detected using a secondary detection reagent, such as an antibody. For example, binding of antibodies in a sample to an array can be detected using a secondary antibody specific for the isotype of an antibody (e.g., IgG (including any of the subtypes, such as IgG1, IgG2, IgG3 and IgG4), IgA, IgM). The secondary antibody is usually labeled and can bind to all antibodies in the sample being analyzed of a particular isotype. Different secondary antibodies (for example, from different hosts) can be used having different isotype specificities.

Binding interactions can also be detected using label-free methods, such as surface plasmon resonance (SPR) and mass spectrometry. SPR can provide a measure of dissociation constants, and dissociation rates, for example, using the A-100 Biocore/GE instrument for this type of analysis.

Detection of binding events can also occur in the presence of competitor peptides. In some embodiments, the competitive inhibitor is a peptide identical to, similar to or derived from a determined epitope, motif or input sequence as disclosed herein. In some embodiments, the competitive inhibitor peptides comprises a mixture of at least 2, at least 3, at least 5, at least 10, at least 15, at least 20, at least 25, at least 30, at least 35, at least 40, at least 45 or at least 50 different peptides. In some embodiments, the competitor peptides comprise natural and/or non-natural amino acids. In some embodiments, the competitive inhibitor peptide comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% and/or at least 99% identical to a determined epitope, motif or input sequence. In other embodiments, the competitive inhibitor peptide comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% and/or at least 99% similar to a determined epitope, motif or input sequence. In some embodiments, the similarity can be determined by sequence or by structure. In other embodiments, the competitive inhibitor peptide may comprise a mixture of random or semi-random peptides. In yet other embodiments, the competitive peptide mixture can include a biological source, for example, serum, plasma or blood, added to or in place of the competitive inhibitor peptides disclosed herein. By adding competitive inhibitor peptides to the binding reaction, and measuring a change in binding signal in the absence and presence of the competitive inhibitor peptides, a measurement of specificity may be obtained that conveys information regarding the stringency of the interaction between peptides on the array and the biological sample. Specificity can be measured in terms of the affinity (Kd) measured in the presence of competitor and/or the number of identified peptides with a determined motif or sequence that bind to the biological sample or antibody and identified as a putative binding site.

### Development and Characterization of Therapeutic Antibodies: Antibody Epitope Binding Profiles

In some embodiments, detection of antibody binding on a peptide array poses some challenges that can be addressed by the technologies disclosed herein. The technologies can tune surface properties with specific coatings and functional group densities, which has been utilized to address two potential shortcomings of using peptide arrays to profile antibody binding. First, non-specific antibody binding on a peptide array is minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation. Also, the surface spacer can be used to cyclize the peptides so that all peptides are presented to the antibody with a consistent ordered structure, compared to linear peptides that are mostly a disordered structure. In some embodiments, the surface spacer includes the following sequence: Cyclic Spacer Ex. 1: Cysteine Glycine-Proline-Glycine-(variable amino acid sequence (Xaa)ₙ)-Glycine-Proline-Glycine-Cysteine, where the two cysteine residues are capable of cyclizing under oxidizing conditions. In other embodiments, the surface spacer may include the following sequence: Cyclic Spacer Ex. 2: Cysteine-(PEG3)-(variable amino acid sequence (Xaa)ₙ)-(PEG3)-Cysteine, where the two cysteine residues are capable of cyclizing under oxidizing conditions. In still other embodiments, the surface spacer includes the following sequence: Cyclic Spacer Ex. 3: Lysine-(PEG3)-(variable amino acid sequence (Xaa)ₙ)-(PEG3)-Lysine, where the two lysine residues are capable of cyclizing with a homobifunctional di-NHS ester linkage. Taken together, these surface developments produce antibody binding profiles on arrays that approach or correlate with solution-phase antibody binding.

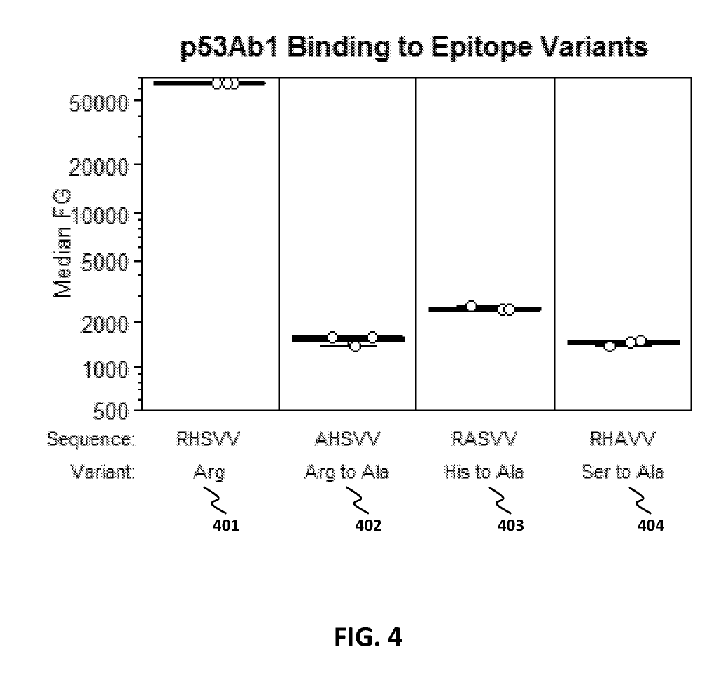

FIG. 4 shows an exemplary embodiment of the methods disclosed herein, depicting alanine scanning of the p53Ab1 monoclonal antibody epitope (RHSVV). An alanine scanning library array was synthesized with alanine individually substituted into the first three positions of the epitope (RHS). Reduced p53Ab1 antibody binding to alanine substitution features (402, 403 and 404) vs. the native epitope (401) on the array developed herein correlate with published p53Ab1 epitope variant binding.

In some embodiments, the technologies disclosed herein address the method of antibody labeling in detection of antibody binding profiles using arrays. Direct fluorescence labeling of antibodies frequently suppresses, modifies or abrogates binding to known epitopes. To address this, the technologies disclosed herein include a “sandwich assay” method, similar to the sandwich ELISA assay, that first binds the unlabeled primary antibody (the antibody being profiled) to the array, which is followed by binding of a fluorescently labeled secondary antibody that binds to a fixed epitope on the unlabeled primary antibody (e.g. the Fc region of IgG antibodies). The binding of the labeled secondary to the primary antibody is validated prior to incubation on the arrays to ensure that the labeled secondary binds the primary antibody as expected.

Validation that the array surface and assay advancements produce a robust antibody binding profile has been performed with an alanine scan of a known peptide epitope (RHSVV) for the p53 binding monoclonal antibody (p53Ab1). The alanine scan peptide sequence set was synthesized using the photolithographic peptide array synthesis and includes: AHSVV, RASVV, RHAVV where alanine (A) is substituted into the first three positions of the epitope. Using the sandwich assay method with the p53AB1 antibody and the alanine scan array, binding to each alanine substitution sequence is compared to the known epitope RHSVV, shown in FIG. 4. The p53Ab1 alanine scan antibody binding profile results obtained from the arrays matches published results that show p53Ab1 requires R, H, and S in the peptide epitope for high-affinity binding.

### Mask Algorithm

The new mask and synthesis algorithm disclosed herein is particularly relevant to antibody discovery and characterization because a target epitope can be used as the input sequence to the algorithm and as a result the region of chemical space surrounding that epitope can be screened, which includes additions, truncations, substitutions and deletions. This is particularly important in screening cancer target antibodies due to the high epitope mutation rates present in cancer. By screening a region of sequence space around the epitope, a large number of cancer-relevant mutations could be detected.

By including a set percent overlap of open features between sequential masks (i.e. mask n vs. mask n+1), a highly diverse chemical library array (e.g., a peptide array) can be synthesized that allows thorough mapping and analysis of the sequence space surrounding an input sequence (e.g., a target epitope).

In some embodiments, a fixed set of photolithographic masks is used to sample the region of chemical space defined by any input sequence up to length n, where n is the number of masks in the set. This algorithm overcomes a major limitation of flexibility in photo-patterned synthesis in that generating a library with a defined sequence typically requires a new set of masks which is expensive and time consuming.

A major innovative outcome is a highly-scalable comprehensive antibody binding characterization platform with the capability to measure the binding profile (i.e. epitope mapping & binning) of at least 500,000 sequence variants derived from any input peptide epitope sequence up to a sequence of amino acids, e.g., up to 2, 3, 4, 5, 6, 7, 8, 9, 10, 11, 12, 13, 14, 15, 16, 147, 18, 19, 20, 21, 22, 23, 24, 25, 26, 27, 28, 29, 30, 31, 32, 33, 34, 35, 36, 37, 38, 39, 40, 50, 60, 70, 80, 90, 100 amino acids in length. The platform methods and devices disclosed herein will significantly advance the field of therapeutic antibody and immunotherapy discovery. The amount and detail of antibody binding information enabled by this new platform can facilitate discovery of new antibody-based therapeutics with novel modes of action and/or minimal off-target side effects that have not yet been achieved. The proposed development facilitate development of multi-specific antibodies that requires characterizing larger numbers of lead candidates with more complex binding profiles vs. mono-specific leads.

In some embodiment, the technologies disclosed herein produce an antibody characterization platform that increases antibody-epitope profile throughput to several million interactions per day, an increase of at least one order of magnitude relative to current platforms. An initial antibody-epitope interaction profile study (e.g. epitope binning) can be performed with a large number of therapeutic antibody candidates (100s of candidates) using the platform described herein.

In some embodiments, a prescriptive photolithographic mask and synthesis algorithm is devised to generate sequence space centered on any input sequence up to length (k), e.g., k=10. In other words, a single set of masks produces a peptide variant library array derived from any input sequence up to 10 amino acids in length. In some embodiments, an input sequence length of 10 is chosen because immunogenic epitope peptides are typically 8-10 amino acids in length. In some embodiments, another length is chosen, e.g., up to 2, 3, 4, 5, 6, 7, 8, 9, 10, 11, 12, 13, 14, 15, 16, 147, 18, 19, 20, 21, 22, 23, 24, 25, 26, 27, 28, 29, 30, 31, 32, 33, 34, 35, 36, 37, 38, 39, 40, 50, 60, 70, 80, 90, 100 amino acids.

In some embodiments, the peptide array sequence space produced by this mask and synthesis algorithm is not limited to a specific input sequence and is defined by: 1) the user-specified input sequence, 2) the order of synthesis steps, 3) the fraction of common open-features between mask n and (n−1), where an open-feature is an array feature that is open for light to pass through on a particular mask n resulting in addition of the next added amino acid at that position.

In some embodiments, the mask and synthesis algorithm disclosed herein is used to iteratively optimize a lead sequence (e.g. peptide) such that a region of sequence space centered around the lead sequence can be screened for higher activity sequences. New leads could be selected and the process repeated iteratively to ultimately identify a peptide sequence with the desired level of activity. In some embodiments, the technologies are utilized for affinity reagents (e.g. peptides) such that the lead peptide(s) affinity could be iteratively improved until reaching the desired affinity or for enzyme activity reagents (e.g. enzyme inhibitors or activators).

In some embodiments, the disclosed photolithographic mask and chemical synthesis algorithm comprises simple and relatively few chemical steps. The algorithm can be thought of the masks and chemical synthesis as a linked combinatory problem such that the sampled space is defined by: 1) an input sequence, 2) the order of synthesis steps and 3) the sequential feature overlap between masks (i.e., overlap between mask n and n−1). This algorithm can be simulated in silico to calculate the size and diversity of the generated space with a defined 1) input sequence, 2) order of chemical steps and 3) percentage sequential mask overlap.

The technology disclosed herein includes an algorithm that utilizes photolithographic masks and the order of synthesis to sample a region of chemical space defined by an input sequence (e.g. peptide sequence) and order of chemical steps. The algorithm determines a set percentage of features (p) that overlap between neighboring sequential masks used for synthesis. The algorithm disclosed herein can be described mathematically. Let p denote a percentage of open features overlapping between mask n and mask n−1; x denote total number of features in the library; ∩ denote intersection, or set of overlapping features (rows, columns); |⋅| denote cardinality, or the number of overlap features. A mask algorithm is: p×x=|(mask n)∩(mask n−1)|.

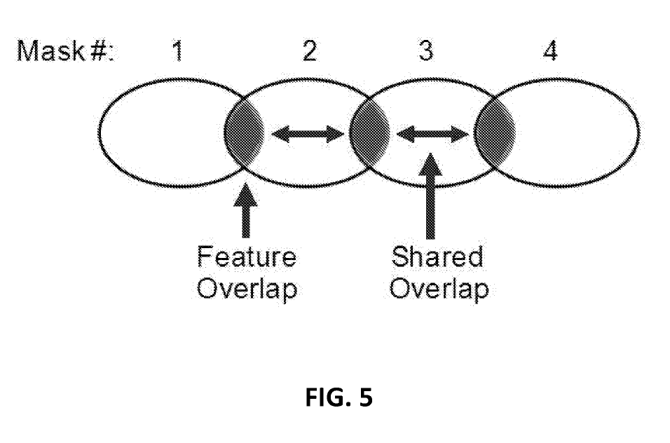

FIG. 5 shows a graphical representation of a mask algorithm. For each mask n, there is a percentage overlap with mask n−1 (a shaded area in FIG. 5). Depending on the scale of percentage overlap, a shared overlap (e.g., shown by a double arrow) may exist between several (or all) sequential masks in the series. This shared overlap can be tuned to define the diversity and median length of the sequences in the sampled space.

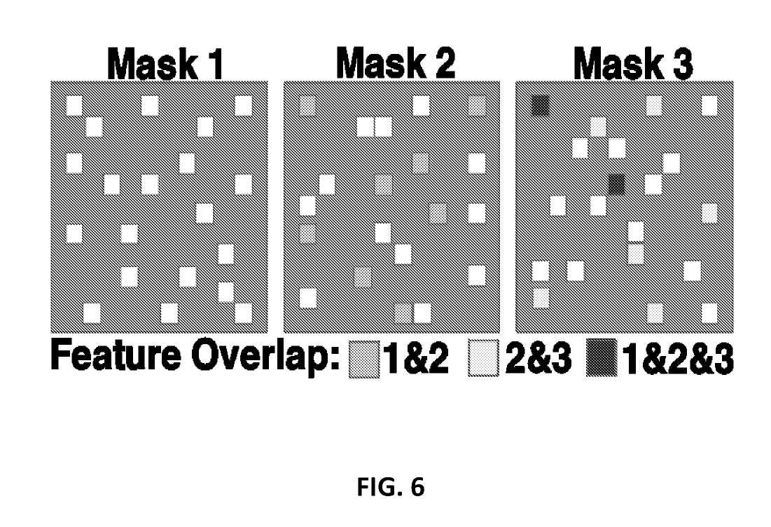

FIG. 6 shows another representation of a mask algorithm. A sequential set of masks are used to selectively expose and activate array features for synthesis. Each mask (n) has fractional feature overlap with mask (n−1). With this algorithm, the maximum possible peptide length in the library is equal to the number of masks and chemical steps used to build the library array, and the median peptide length of the library is dependent on the fraction of open-feature overlap between mask ⁿ and (n−1).

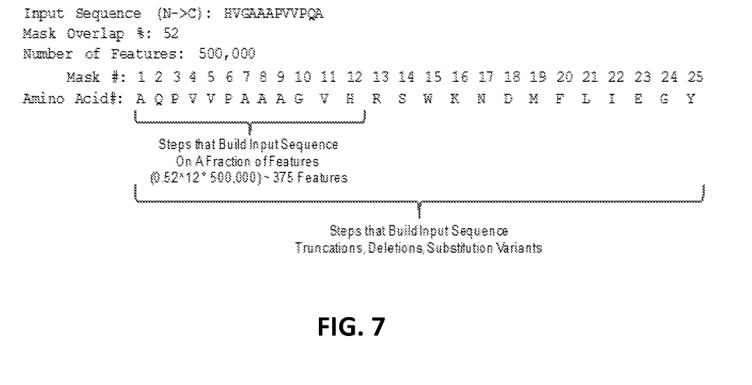

FIG. 7 shows a graphical representation of some embodiments with ordered synthesis steps. In FIG. 7, the order of amino acid coupling is based on the input sequence. In this example the 12-mer input sequence, HVGAAAPVVPQA, is built on a fraction of the total features in the first 12 steps, where each step is corresponding to a mask number. The region of sampled sequence space around this input sequence is generated from the following: (a) an order of amino acids, (b) a ratio of overlapping and non-overlapping features in steps 1-12, and (c) a percent of overlapping features and amino acids used in steps 13-25. Other examples of synthesis order exist where amino acids not present in the input sequence are interleaved with the input amino acid sequence (see specific embodiment section that follows). The disclosed algorithm has flexibility to rationally focus the sequence space or even to sample random sequence space (i.e. the masks and order of synthesis steps are randomized).

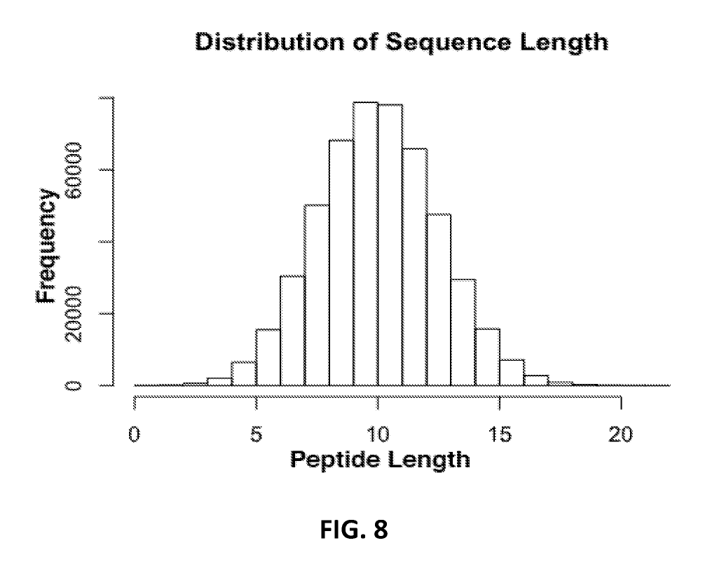

In an exemplary embodiment, a total of 25 photolithographic masks are designed and produced to accommodate chemical steps that include all 20 natural amino acids in peptide library array synthesis. Photolithographic mask array features will be patterned at 18 μm pitch to produce 500,000 total array features after 25 synthesis steps, with 8 arrays per 75 mm×25 mm slide, for a total of 4,000,000 features per slide (1 slide=8 replicate arrays of 500,000 features per array=4,000,000 features/slide). The fraction of open-feature overlap between mask n and (n−1) in the series of masks will be set to 42% to achieve a median peptide library array length equal to the input sequence length of 10. Each mask will have 210,000 open-features selected randomly from the total of 500,000 array features where 42% of the open-features on mask n overlap with mask (n−1). This fraction overlap was determined from an in silico simulated synthesis using a therapeutic antibody 10-mer epitope input peptide sequence (QMWAPQWGPD, a Herceptin therapeutic antibody epitope [57]) to generate a library of 458,305 distinct sequence variants and 41,695 replicated sequence variants (500,000 total features) from 25 photolithographic synthesis steps. FIG. 8 shows a distribution of peptide lengths from in silico simulated peptide library synthesis using the prescriptive mask algorithm illustrated in FIG. 6 with a 10-mer input sequence and 25 Synthesis steps, where the median length is 10.

### Mass Spectrometry Detection

In some embodiments, the technologies disclosed herein develop in situ mass spectrometry detection of a set of peptide sequences on each chip that interrogate every synthesis step to quantify the efficiency and purity of each step. The technologies build on initial MALDI development to enable yield and purity quantitation. In some embodiments, in situ MALDI mass spectra are acquired from the synthesized peptide array by incorporating a gas-phase cleavable, safety-catch linker (SCL) that is stable to binding assay conditions and can be cleaved without diffusion from the silicon surface using ammonia gas. The SCL will be coupled to the amine functionalized silicon surface and peptides will be built from the SCL surface linkage. After peptide array synthesis on an 8-inch wafer, 13 microscope slide dimensioned chips with 8 replicate peptide arrays per chip, are diced from the wafer and one chip reserved for MALDI mass spectra acquisition. Ammonia gas treatment of the MALDI reserved chip cleaves the synthesized peptide from the silicon surface without diffusion. Following gas-phase cleavage, a MALDI matrix that facilitates peptide desorption/ionization is applied to the chip using microdroplet aerosol application without diffusion of the cleaved peptides on the array surface. Finally, MALDI mass spectra are acquired in situ from the synthesized peptide array by aligning the MALDI laser to specific cleaved peptide array features relative to a set of alignment fiducial markers to ensure the laser is centered on the intended array feature for mass spectrum acquisition.

In some embodiments, to quantify the efficiency of every synthesis step with MALDI mass spectrometry, a set of 500 μm² MALDI synthesis-analysis array features is included on the masks (e.g., aforementioned 25 masks) produced. A total of 25 MALDI analysis array features corresponding to each of the 25 synthesis steps are patterned on all 13 chips within an 8-inch wafer, enabling efficiency calculation for all steps in the combinatorial synthesis. A common C-terminal (first synthesis position) amino acid (e.g. glycine) is coupled to all MALDI analysis array features as the first synthetic step. Following the common amino acid, each individual MALDI analysis feature is photodeprotected in series with each of the 25 array synthesis masks. The corresponding amino acid for that synthesis step is coupled to the photodeprotected MALDI analysis feature to produce a dimer sequence consisting of the amino acid for that synthesis step coupled to the common amino acid (e.g. arginine-glycine dimer). To normalize MALDI ionization across all peptide sequences, tris(2,4,6-trimethoxyphenyl)phosphonium (TMPP) signal enhancer is coupled to all N-termini. After MALDI mass spectrum data acquisition from all 25 features, the efficiency of each synthesis step will be calculated as a ratio of the mass spectrum peaks of the desired dimer vs. the common monomer (e.g. arginine-glycine vs. glycine).

### Binding Profile Reproducibility

In various embodiments, the technologies disclosed herein includes quantifying intra- and inter-array binding profile reproducibility with a set of 5 engineered antibodies and confirm binding profiles with peptide resynthesis and surface plasmon resonance (SPR).

In some embodiments, a set of 5 monoclonal antibodies and 5 separate arrays are used to quantify antibody binding profile reproducibility (i.e. % CV). By using a defined set of antibodies, antibody concentration and sample composition can be tightly controlled to measure variability of the array production vs. variability in the samples or assay.

In an exemplary embodiment to test the binding profile reproducibility obtained, five unrelated peptide epitopes with lengths in the range of 6-10 amino acids will be identified from literature and used as input sequences for 5 separate peptide array syntheses of epitope variant libraries. Five IgG monoclonal antibodies engineered to bind the selected epitopes are used. Each of the five antibodies is bound separately to their respective variant library array. Primary antibody binding is labeled using a fluorescently labeled anti-IgG Fc secondary antibody that binds to the Fc region of the primary IgG antibody based on a sandwich assay protocol. Intra-array % CVs will be calculated using replicate peptide feature fluorescence intensities within one array. Inter-array % CVs will be calculated using identical feature fluorescence intensities on replicate arrays. Five epitope variant sequences are selected from each of the five antibody array binding profiles (25 total peptides) for synthesis and purification followed by solution-phase SPR binding analysis.

### Digital Processing Device

In some embodiments, the systems, platforms, software, networks, and methods described herein include a digital processing device, or use of the same. In further embodiments, the digital processing device includes one or more hardware central processing units (CPUs), i.e., processors that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280× about 720 pixels or at least about 1920× about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

### Non-Transitory Computer Readable Storage Medium

In some embodiments, the systems, platforms, software, networks, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Kits.

Devices and methods of the disclosed embodiments can be packaged as a kit. In some embodiments, a kit includes written instructions on the use of the device or methods. The written material can be, for example, a label. The written material can suggest conditions, for example, for an assay or steps to perform an assay. The instructions provide the user with the best guidance for using the devices and/or performing the methods and assays disclosed herein.

### EMBODIMENTS

The following non-limiting embodiments provide illustrative examples of the invention, but do not limit the scope of the invention.

### Embodiment 1

In some embodiments, provide herein are methods of in situ synthesizing a chemical library on a substrate, the chemical library comprising a plurality of molecules, the method comprising:
(a) receiving a biological sequence and a number of synthesis steps;
(b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask;
(c) assigning at least one monomer to each patterned mask; and
(d) coupling the monomers onto the features to form molecules;
(e) wherein (c) and (d) assembles one said synthesis step and the synthesis step is repeated.

### Embodiment 2

The method of Embodiment 1, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

### Embodiment 3

The method of Embodiment 1, wherein the input biological sequence comprises a disease-related epitope.

### Embodiment 4

The method of Embodiment 1, wherein the input biological sequence comprises a disease-related epitope.

### Embodiment 5

The method of Embodiment 1, wherein the input biological sequence comprises a peptide sequence.

### Embodiment 6

The method of Embodiment 1, wherein the input biological sequence comprises an epitope sequence

### Embodiment 7

The method of Embodiment 1, wherein the input biological sequence comprises a random sequence

### Embodiment 8

The method of Embodiment 1, further comprising deriving an ordered list of monomers from the input biological sequence.

### Embodiment 9

The method of Embodiment 8, wherein a size of the ordered list is the number of the synthesis steps.

### Embodiment 10

The method of Embodiment 8, wherein the ordered list of monomers comprises the input biological sequence.

### Embodiment 11

The method of Embodiment 10, wherein the ordered list of monomers comprises the input biological sequence in a reversed order.

### Embodiment 12

The method of Embodiment 8, wherein the molecules are peptides or nucleic acids.

### Embodiment 13

The method of Embodiment 8, wherein the ordered list of monomers comprises a sequence of amino acids.

### Embodiment 14

The method of Embodiment 8, wherein the ordered list of monomers comprises a sequence of nucleotides.

### Embodiment 15

The method of Embodiment 1, wherein a number of the plurality of the patterned masks is less than 10, 15, 20, 25, 30, 35, 40, 45, 50, 60, 70, 80, 90, or 100.

### Embodiment 16

The method of Embodiment 1, wherein a number of the plurality of the patterned masks is the number of the synthesis steps.

### Embodiment 17

The method of Embodiment 1, wherein about 20% to about 50% of the activated designation features in each sequential patterned mask overlaps with the activated designation features of an immediately preceding patterned mask.

### Embodiment 18

The method of Embodiment 1, wherein about 30% to about 45% of the activated designation features in each sequential patterned mask overlaps with the activated designation features of an immediately preceding patterned mask.

### Embodiment 19

The method of Embodiment 1, wherein the synthesis step is based on photolithography.

### Embodiment 20

The method of Embodiment 1, wherein a feature on the substrate is about 0.5 micron to about 200 microns in diameter and a center-to-center distance of about 1 micron to about 300 microns on center.

### Embodiment 21

The method of Embodiment 1, wherein at least 40% of the molecules, at least 50% of the molecules, at least 60% of the molecule, at least 70% of the molecules, at least 80% of the molecules or at least 90% of the molecules in the library are distinct.

### Embodiment 22

The method of Embodiment 1, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 23

The method of Embodiment 1, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length

### Embodiment 24

The method of Embodiment 1, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

### Embodiment 25

The method of Embodiment 1, wherein the library comprises a median monomer length equal to a length of the biological sequence.

### Embodiment 26

The method of Embodiment 1, wherein the library comprises a median monomer length longer than 40%, 50%, 60%, 70%, 80%, or 90% of a length of the biological sequence.

### Embodiment 27

The method of Embodiment 1, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

### Embodiment 28

The method of Embodiment 1, wherein the substrate is selected from the group consisting of arrays, wafers, slides and beads.

### Embodiment 29

The method of Embodiment 1, wherein the synthesized chemical library comprises peptides, nucleotides or a combination thereof.

### Embodiment 30

The method of Embodiment 29, wherein the peptides are about 5 to about 25 amino acids in length.

### Embodiment 31

The method of Embodiment 29, wherein the amino acids C, I, and M, and optionally Q and E, are not included in the amino acids available for peptide synthesis.

### Embodiment 32

The method of Embodiment 1, wherein the chemical library is synthesized with a surface spacer capable of cyclizing under oxidizing conditions.

### Embodiment 33

The method of Embodiment 32, wherein the surface spacer is Cys-Gly-Pro-Gly-XaaₙGly-Pro-Gly-Cys or Cys-(PEG3)-Xaaₙ-(PEG3)-Cys.

### Embodiment 34

The method of Embodiment 1, wherein the chemical library is synthesized with a surface spacer capable of cyclizing with an ester linkage.

### Embodiment 35

The method of Embodiment 34, wherein the ester linkage is a homobifunctional di-NHS ester linkage.

### Embodiment 36

The method of Embodiment 34, wherein the surface spacer is Lys-(PEG3)-Xaaₙ-(PEG3)-Lysine.

### Embodiment 37

The method of Embodiment 1, wherein the substrate is coated with a hydrophilic monolayer

### Embodiment 38

The method of Embodiment 37, wherein the hydrophilic monolayer comprises polyethylene glycol (PEG), polyvinyl alcohol, carboxymethyl dextran, and combinations thereof.

### Embodiment 39

The method of Embodiment 37, wherein the hydrophilic monolayer is homogeneous.

### Embodiment 40

In some embodiments, provided herein are in situ synthesized chemical libraries, the chemical library comprising a plurality of molecules, wherein the synthesis uses patterned steps to construct the library on a substrate, comprising:
(a) receiving a biological sequence and a number of synthesis steps;
(b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask;
(c) assigning at least one monomer to each patterned mask; and
(d) coupling the monomers onto the features to form molecules; wherein (c) and (d) assembles one said synthesis step and the synthesis step is repeated.

### Embodiment 41

The chemical library of Embodiment 40, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

### Embodiment 42

The chemical library of Embodiment 40, wherein the input biological sequence comprises a disease-related epitope, a peptide sequence, an epitope sequence and/or a random sequence.

### Embodiment 43

The chemical library of Embodiment 40, further comprising deriving an ordered list of monomers from the input biological sequence.

### Embodiment 44

The chemical library of Embodiment 43, wherein a size of the ordered list is the number of the synthesis steps.

### Embodiment 45

The chemical library of Embodiment 43, wherein the ordered list of monomers comprises the input biological sequence.

### Embodiment 46

The chemical library of Embodiment 43, wherein the ordered list of monomers comprises the input biological sequence in a reversed order.

### Embodiment 47

The chemical library of Embodiment 40, wherein the molecules comprise peptides or nucleic acids.

### Embodiment 48

The chemical library of Embodiment 43, wherein the ordered list of monomers comprises a sequence of amino acids and/or a sequence of nucleotides.

### Embodiment 49

The chemical library of Embodiment 40, wherein a number of the plurality of the patterned masks is less than 10, 15, 20, 25, 30, 35, 40, 45, 50, 60, 70, 80, 90, or 100.

### Embodiment 50

The chemical library of Embodiment 40, wherein a number of the plurality of the patterned masks is the number of the synthesis steps.

### Embodiment 51

The chemical library of Embodiment 40, wherein about 20% to about 50%, or about 30% to about 45% of the activated designation features in each sequential patterned mask overlaps with the activated designation features of an immediately preceding patterned mask.

### Embodiment 52

The chemical library of Embodiment 40, wherein the synthesis step is based on photolithography.

### Embodiment 53

The chemical library of Embodiment 40, wherein a feature on the substrate is about 0.5 micron to about 200 microns in diameter and a center-to-center distance of about 1 micron to about 300 microns on center.

### Embodiment 54

The chemical library of Embodiment 40, wherein at least 40%, at least 50%, at least 60%, at least 70%, at least 80% or at least 90% of the molecules in the library are distinct.

### Embodiment 55

The chemical library of Embodiment 40, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 56

The chemical library of Embodiment 40, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 57

The chemical library of Embodiment 40, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

### Embodiment 58

The chemical library of Embodiment 40, wherein the library comprises a median monomer length equal to a length of the biological sequence.

### Embodiment 59

The chemical library of Embodiment 40, wherein the library comprises a median monomer length longer than 40%, 50%, 60%, 70%, 80%, or 90% of a length of the biological sequence.

### Embodiment 60

The chemical library of Embodiment 40, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

### Embodiment 61

The chemical library of Embodiment 40, wherein the substrate is selected from the group consisting of arrays, wafers, slides and beads.

### Embodiment 62

The chemical library of Embodiment 40, wherein the synthesized chemical library comprises peptides, nucleotides or a combination thereof.

### Embodiment 63

The chemical library of Embodiment 62, wherein the peptides are about 5 to about 25 amino acids in length.

### Embodiment 64

The chemical library of Embodiment 63, wherein the amino acids C, I, and M, and optionally Q and E, are not included in the amino acids available for peptide synthesis.

### Embodiment 65

The chemical library of Embodiment 40, wherein the chemical library is synthesized with a surface spacer capable of cyclizing under oxidizing conditions.

### Embodiment 66

The chemical library of Embodiment 65, wherein the surface spacer is Cys-Gly-Pro-Gly-Xaaₙ-Gly-Pro-Gly-Cys or Cys-(PEG3)-Xaaₙ-(PEG3)-Cys.

### Embodiment 67

The chemical library of Embodiment 40, wherein the chemical library is synthesized with a surface spacer capable of cyclizing with an ester linkage.

### Embodiment 68

The chemical library of Embodiment 67, wherein the ester linkage is a homobifunctional di-NHS ester linkage.

### Embodiment 69

The chemical library of Embodiment 68, wherein the surface spacer is Lys-(PEG3)-Xaaₙ-(PEG3)-Lysine.

### Embodiment 70

The chemical library of Embodiment 40, wherein the substrate is coated with a hydrophilic monolayer.

### Embodiment 71

The chemical library of Embodiment 70, wherein the hydrophilic monolayer comprises polyethylene glycol (PEG), polyvinyl alcohol, carboxymethyl dextran, and combinations thereof.

### Embodiment 72

The chemical library of Embodiment 70, wherein the hydrophilic monolayer is homogeneous.

### Embodiment 73

In some embodiments, provided herein are computing systems for simulating in situ synthesis of a chemical library on a substrate, the chemical library comprising a plurality of molecules, comprising:
(a) a processor and a memory;
(b) a computer program including instructions executable by the processor, the computer program comprising:
   (1) a receiving module configured to receive a biological sequence and a number of synthesis steps;
   (2) a simulation module configured to: (i) determine a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask; (ii) assign at least one monomer to each patterned mask; and (iii) couple the monomers onto the features to form molecules; wherein (i), (ii) and (iii) assembles one said synthesis step and the synthesis step is repeated.

### Embodiment 74

The system of Embodiment 73, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

### Embodiment 75

The system of Embodiment 73, wherein the input biological sequence comprises a disease-related epitope, a peptide sequence, an epitope sequence, and/or a random sequence.

### Embodiment 76

The system of Embodiment 73, further comprising deriving an ordered list of monomers from the input biological sequence.

### Embodiment 77

The system of Embodiment 76, wherein a size of the ordered list is the number of the synthesis steps.

### Embodiment 78

The system of Embodiment 76, wherein the ordered list of monomers comprises the input biological sequence.

### Embodiment 79

The system of Embodiment 78, wherein the ordered list of monomers comprises the input biological sequence in a reversed order.

### Embodiment 80

The system of Embodiment 73, wherein the molecules comprises peptides or nucleic acids.

### Embodiment 81

The system of Embodiment 73, wherein the ordered list of monomers comprises a sequence of amino acids and/or a sequence of nucleotides.

### Embodiment 82

The system of Embodiment 73, wherein a number of the plurality of the patterned masks is less than 10, 15, 20, 25, 30, 35, 40, 45, 50, 60, 70, 80, 90, or 100.

### Embodiment 83

The system of Embodiment 73, wherein a number of the plurality of the patterned masks is the number of the synthesis steps.

### Embodiment 84

The system of Embodiment 73, wherein about 20% to about 50%, or about 30% to about 45% of the activated designation features in each sequential patterned mask overlaps with the activated designation features of an immediately preceding patterned mask.

### Embodiment 85

The system of Embodiment 73, wherein the synthesis step is based on photolithography.

### Embodiment 86

The system of Embodiment 73, wherein a feature on the substrate is about 0.5 micron to about 200 microns in diameter and a center-to-center distance of about 1 micron to about 300 microns on center.

### Embodiment 87

The system of Embodiment 73, wherein at least 40%, at least 50%, at least 60%, at least 70%, at least 80% or at least 90% of the molecules in the library are distinct.

### Embodiment 88

The system of Embodiment 73, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 89

The system of Embodiment 73, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length

### Embodiment 90

The system of Embodiment 73, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

### Embodiment 91

The system of Embodiment 73, wherein the library comprises a median monomer length equal to a length of the biological sequence.

### Embodiment 92

The system of Embodiment 73, wherein the library comprises a median monomer length longer than 40%, 50%, 60%, 70%, 80%, or 90% of a length of the biological sequence.

### Embodiment 93

The system of Embodiment 73, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

### Embodiment 94

The system of Embodiment 73, wherein the substrate is selected from the group consisting of arrays, wafers, slides and beads.

### Embodiment 95

The system of Embodiment 73, wherein the synthesized chemical library comprises peptides, nucleotides or a combination thereof.

### Embodiment 96

The system of Embodiment 95, wherein the peptides are about 5 to about 25 amino acids in length

### Embodiment 97

The system of Embodiment 96, wherein the amino acids C, I, and M, and optionally Q and E, are not included in the amino acids available for peptide synthesis.

### Embodiment 98

The system of Embodiment 73, wherein the chemical library is synthesized with a surface spacer capable of cyclizing under oxidizing conditions.

### Embodiment 99

The system of Embodiment 98, wherein the surface spacer is Cys-Gly-Pro-Gly-Xaaₙ-Gly-Pro-Gly-Cys or Cys-(PEG3)-Xaaₙ-(PEG3)-Cys.

### Embodiment 100

The system of Embodiment 73, wherein the chemical library is synthesized with a surface spacer capable of cyclizing with an ester linkage.

Embodiment 101 The system of Embodiment 100, wherein the ester linkage is a homobifunctional di-NHS ester linkage.

### Embodiment 102

The system of Embodiment 101, wherein the surface spacer is Lys-(PEG3)-Xaaₙ-(PEG3)-Lysine.

### Embodiment 103

The system of Embodiment 73, wherein the substrate is coated with a hydrophilic monolayer.

### Embodiment 104

The system of Embodiment 103, wherein the hydrophilic monolayer comprises polyethylene glycol (PEG), polyvinyl alcohol, carboxymethyl dextran, and combinations thereof.

### Embodiment 105

The system of Embodiment 103, wherein the hydrophilic monolayer is homogeneous.

### Embodiment 106

In some embodiments, provided herein are methods for in situ synthesizing a peptide array, the method comprising
(a) receiving an input amino acid sequence;
(b) determining a number of synthesis steps;
(c) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
(d) assigning at least one monomer to each patterned mask; and
(e) coupling the monomers onto the features, wherein (c) and (d) assembles one said synthesis step and said synthesis step is repeated to form the peptide array.

### Embodiment 107

The method of Embodiment 106, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

### Embodiment 108

The method of Embodiment 106, wherein the input sequence comprises a disease-related epitope, a peptide sequence or an epitope sequence.

### Embodiment 109

The method of Embodiment 106, further comprising deriving an ordered list of monomers from the input sequence.

### Embodiment 110

The method of Embodiment 109, wherein a size of the ordered list is the number of the synthesis steps.

### Embodiment 111

The method of Embodiment 109, wherein the ordered list of monomers comprises the input sequence.

### Embodiment 112

The method of Embodiment 111, wherein the ordered list of monomers comprises the input sequence in a reversed order.

### Embodiment 113

The method of Embodiment 109, wherein the ordered list of monomers comprises a sequence of amino acids.

### Embodiment 114

The method of Embodiment 106, wherein a number of the plurality of the patterned masks is less than 10, 15, 20, 25, 30, 35, 40, 45, 50, 60, 70, 80, 90, or 100.

### Embodiment 115

The method of Embodiment 106, wherein a number of the plurality of the patterned masks is the number of the synthesis steps.

### Embodiment 116

The method of Embodiment 106, wherein about 20% to about 50%, or about 30% to about 45% of the activated designation features in each sequential patterned mask overlaps with the activated designation features of an immediately preceding patterned mask.

### Embodiment 117

The method of Embodiment 106, wherein the synthesis step is based on photolithography.

### Embodiment 118

The method of Embodiment 106, wherein a feature on the substrate is about 0.5 micron to about 200 microns in diameter and a center-to-center distance of about 1 micron to about 300 microns on center.

### Embodiment 119

The method of Embodiment 106, wherein at least 40%, at least 50%, at least 60%, at least 70%, at least 80% or at least 90% of the peptides on the array are distinct.

### Embodiment 120

The method of Embodiment 106, wherein at least 50% of the peptides on the array are at least 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 121

The method of Embodiment 106, wherein at least 50% of the peptides on the array are at most 3, 4, 5, 6, 7, 8, 9, 10, 11, 12, 13, 14, 15, 16, 17, 18, 19, 20, 30, 40, 50, 60, 70, 80, 90, or 100 monomers in length.

### Embodiment 122

The method of Embodiment 106, wherein the peptides on the array comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

### Embodiment 123

The method of Embodiment 106, wherein the array comprises a median peptide length equal to a length of the input sequence.

### Embodiment 124

The method of Embodiment 106, wherein the array comprises a median peptide length longer than 40%, 50%, 60%, 70%, 80%, or 90% of a length of the input sequence.

### Embodiment 125

The method of Embodiment 106, wherein the array comprises a median peptide length shorter than 60%, 70%, 80%, 90%, 100%, 110%, 120%, 130%, 140%, 150%, 160%, 170%, 180%, 190%, or 200% of a length of the input sequence.

### Embodiment 126

The method of Embodiment 106, wherein the peptides are about 5 to about 25 amino acids in length.

### Embodiment 127

The method of Embodiment 106, wherein the amino acids C, I, and M, and optionally Q and E, are not included in the amino acids available for peptide synthesis.

### Embodiment 128

The method of Embodiment 106, wherein the peptide array is synthesized with a surface spacer capable of cyclizing under oxidizing conditions.

### Embodiment 129

The method of Embodiment 128, wherein the surface spacer is Cys-Gly-Pro-Gly-Xaaₙ-Gly-Pro-Gly-Cys or Cys-(PEG3)-Xaaₙ-(PEG3)-Cys.

### Embodiment 130

The method of Embodiment 106, wherein the peptide array is synthesized with a surface spacer capable of cyclizing with an ester linkage.

### Embodiment 131

The method of Embodiment 130, wherein the ester linkage is a homobifunctional di-NHS ester linkage.

### Embodiment 132

The method of Embodiment 130, wherein the surface spacer is Lys-(PEG3)-Xaaₙ-(PEG3)-Lysine.

### Embodiment 133

The method of Embodiment 106, wherein the peptide array is coated with a hydrophilic monolayer.

### Embodiment 134

The method of Embodiment 132, wherein the hydrophilic monolayer comprises polyethylene glycol (PEG), polyvinyl alcohol, carboxymethyl dextran, and combinations thereof.

### Embodiment 135

The method of Embodiment 132, wherein the hydrophilic monolayer is homogeneous

### Embodiment 136

In some embodiments included herein are arrays comprising a plurality of in situ synthesized peptides on the array, the peptides produced by a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask.

### Embodiment 137

In some embodiments included herein are methods for characterizing antibody binding against at least one protein target, the method comprising
contacting a peptide array with said antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides;
(b) aligning the individual peptides to said at least one protein target, wherein the alignments between the individual peptides of step (a) and at least one protein target are assigned alignment scores; and
(c) characterizing binding of the antibody against the at least one protein target using the alignment scores of step (b).

### Embodiment 138

The method of Embodiment 137, wherein the predetermined threshold is a binding signal in the presence of competitor peptides within at least 20-fold of the binding signal in the absence of competitor peptides.

### Embodiment 139

The method of Embodiment 137, wherein the predetermined threshold is a binding signal in the presence of competitor peptides of at least 5% of the binding signal as compared in the absence of competitor.

### Embodiment 140

The method of Embodiment 137, wherein the competitor peptides comprise a biological sample.

### Embodiment 141

The method of Embodiment 137, wherein the biological sample is serum.

### Embodiment 142

The method of Embodiment 137, wherein the competitor peptides are derived from the target protein.

### Embodiment 143

The method of Embodiment 142, wherein the competitor peptides are at least 50% similar to the target protein.

### Embodiment 144

The method of Embodiment 137, wherein the competitor peptides are derived from a known epitope of the antibody.

### Embodiment 145

The method of Embodiment 144, wherein the competitor peptides are at least 50% similar to the known epitope of the antibody.

### Embodiment 146

The method of Embodiment 137, wherein the competitor peptides comprise a biological sample and a peptide of any of Embodiments 142 to 145.

### Embodiment 147

The method of Embodiment 137, wherein the peptide array comprises at least 1000, at least 10,000, at least 100,000 or at least 1,000,000 unique peptides.

### Embodiment 148

The method of Embodiment 137, wherein the peptide array is in situ synthesized.

### Embodiment 149

The method of Embodiment 148, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

### Embodiment 150

The method of Embodiment 137, wherein the binding signal is measured as an intensity of the signal in the absence and presence of the competitor peptides at one or more concentrations.

### Embodiment 151

The method of Embodiment 137, wherein an apparent Kd is obtained in the presence and absence of the competitor peptides at one or more concentrations.

### Embodiment 152

The method of Embodiment 137, wherein at least one additional antibody is contacted with the peptide array, and the alignment scores obtained with each antibody are ranked to determine the propensity of each antibody to bind to the protein target

### Embodiment 153

The method of Embodiment 137, further comprising determining a metric score for each antibody, wherein each antibody is assigned a single binding profile metric derived from the combination of the alignment scores from step (b) in claim 169 and the signal of the individual peptides of step (a) with more than one aligned position from step (b).

### Embodiment 154

The method of Embodiment 137, further comprising determining a metric score for each antibody, wherein each antibody is assigned a single specificity profile metric derived from the combination of the alignment scores from step (b) in claim 169, the number of peptides with more than one aligned position from step (b) and the signal of the individual peptides of step (a) with more than one aligned position from step (b).

### Embodiment 155

In some embodiments, disclosed herein are methods for identifying an antibody epitope in a target protein, the method comprising:
(a) contacting a peptide array with said antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the individual peptides to said at least one protein target, wherein the alignments between the individual peptides of step (a) and at least one protein target are assigned alignment scores; and
(c) determining conserved amino acids in the individual peptides of step (a) to identify a conserved binding peptide motif and aligning the individual motifs to said at least one target protein in order to identify at least one antibody epitope of the target protein.

### Embodiment 156

The method of Embodiment 155, wherein the predetermined threshold is a binding signal in the presence of competitor peptides within at least 20-fold of the binding signal in the absence of competitor peptides.

### Embodiment 157

The method of Embodiment 155, wherein the predetermined threshold is a binding signal in the presence of competitor peptides wherein the predetermined threshold is a binding signal in the presence of competitor peptides of at least 5% of the binding signal as compared in the absence of competitor.

### Embodiment 158

The method of Embodiment 155, wherein the competitor peptides comprise a biological sample.

### Embodiment 159

The method of Embodiment 155, wherein the biological sample is serum.

### Embodiment 160

The method of Embodiment 155, wherein the competitor peptides are derived from the target protein.

### Embodiment 161

The method of Embodiment 160, wherein the competitor peptides are at least 50% similar to the target protein.

### Embodiment 162

The method of Embodiment 155, wherein the competitor peptides are derived from a known epitope of the antibody.

### Embodiment 163

The method of Embodiment 162, wherein the competitor peptides are at least 50% similar to the known epitope of the antibody.

### Embodiment 164

The method of Embodiment 155, wherein the competitor peptides comprise a biological sample and a peptide of any of Embodiments 160 to 163.

### Embodiment 165

The method of Embodiment 155, wherein the peptide array comprises at least 1000, at least 10,000, at least 100,000 or at least 1,000,000 unique peptides.

### Embodiment 166

The method of Embodiment 155, wherein the peptide array is in situ synthesized.

### Embodiment 167

The method of Embodiment 166, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

### Embodiment 168

The method of Embodiment 155, wherein the binding signal is measured as an intensity of the signal in the absence and presence of the competitor peptides at one or more concentrations.

### Embodiment 169

The method of Embodiment 155, wherein an apparent Kd is obtained in the presence and absence of the competitor peptides at one or more concentrations

### Embodiment 170

The method of Embodiment 155, wherein at least one additional antibody is contacted with the peptide array, and the alignment scores obtained with each antibody are ranked to determine the propensity of each antibody to bind to the protein target.

### Embodiment 171

The method of Embodiment 155, further comprising determining a metric score for each antibody, wherein each antibody is assigned a single binding profile metric derived from the combination of the alignment scores from step (b) in claim 190 and the signal of the individual peptides of step (a) with more than one aligned position from step (b).

### Embodiment 172

The method of Embodiment 155, further comprising determining a metric score for each antibody, wherein each antibody is assigned a single specificity profile metric derived from the combination of the alignment scores from step (b) in claim 190, the number of peptides with more than one aligned position from step (b) and the signal of the individual peptides of step (a) with more than one aligned position from step (b).

### Embodiment 173

The method of Embodiment 155, further comprising aligning the at least one antibody epitope as a search criteria against a protein database.

### Embodiment 174

The method of Embodiment 173, wherein the protein database is a proteome database and wherein additional antibody target proteins and/or cross-reactive proteins are identified.

### Embodiment 175

In some embodiments disclosed herein are methods for characterizing antibody binding regions in a target protein, the method comprising:
(a) contacting a first peptide array with said antibody in the presence and absence of a plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides;
(b) creating a second peptide array using an input peptide sequence chosen from at least one of the individual peptides in step (a), a conserved motif derived from an alignment of the individuals peptides in step (a) or an aligned motif derived from an alignment of the individual peptides in step (a), the second peptide array synthesized by:
   i. determining a number of synthesis steps;
   ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
   iii. assigning at least one monomer to each patterned mask; and
   iv. coupling the monomers onto the features, wherein (ii) and (iii) assembles one said synthesis step and said synthesis step is repeated to form the peptide array;

### Embodiment 176

The method of Embodiment 175, wherein the competitor peptides comprise a biological sample.

### Embodiment 177

The method of Embodiment 175, wherein the biological sample is serum

### Embodiment 178

The method of Embodiment 175, wherein the competitor peptides are derived from the target protein.

### Embodiment 179

The method of Embodiment 178, wherein the competitor peptides are at least 50% similar to the target protein.

### Embodiment 180

The method of Embodiment 175, wherein the competitor peptides are derived from a known epitope of the antibody.

### Embodiment 181

The method of Embodiment 180, wherein the competitor peptides are at least 50% similar to the known epitope of the antibody.

### Embodiment 182

The method of Embodiment 175, wherein the competitor peptides comprise a biological sample and a peptide of any of Embodiments 178 to 181.

### Embodiment 183

The method of Embodiment 175, wherein the peptide array comprises at least 1000, at least 10,000, at least 100,000 or at least 1,000,000 unique peptides.

### Embodiment 184

The method of Embodiment 175, wherein the peptide array is in situ synthesized.

### Embodiment 185

The method of Embodiment 175, wherein the first peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

### Embodiment 186

The method of Embodiment 175, wherein the binding signal is measured as an intensity of the signal in the absence and presence of the competitor peptides at one or more concentrations.

### Embodiment 187

The method of Embodiment 175, wherein an apparent Kd is obtained in the presence and absence of the competitor peptides at one or more concentrations.

### Embodiment 188

The method of Embodiment 175, wherein at least one additional antibody is contacted with the peptide array, and the alignment scores obtained with each antibody are ranked to determine the propensity of each antibody to bind to the protein target.

### Embodiment 189

The method of Embodiment 175, further comprising determining a metric score for each antibody, wherein each antibody is assigned a single binding profile metric derived from the combination of the alignment scores from step (b) in Embodiment 175 and the signal of the individual peptides of step (a) with more than one aligned position from step (b).

### Embodiment 190

The method of Embodiment 175, further comprising determining a metric score for each antibody, wherein each antibody is assigned a single specificity profile metric derived from the combination of the alignment scores from step (b) in claim 213, the number of peptides with more than one aligned position from step (b) and the signal of the individual peptides of step (a) with more than one aligned position from step (b).

### Embodiment 191

The method of Embodiment 175, further comprising aligning the at least one antibody epitope as a search criteria against a protein database.

### Embodiment 192

The method of Embodiment 191, wherein the protein database is a proteome database and wherein additional antibody target proteins and/or cross-reactive proteins are identified.

### Embodiment 193

The method of Embodiment 175, wherein the first predetermined threshold is a binding signal in the presence of competitor peptides within at least 20-fold of the binding signal in the absence of competitor peptides.

### Embodiment 194

The method of Embodiment 175, wherein the second predetermined threshold is a binding signal in the presence of competitor peptides within at least 20-fold of the binding signal in the absence of competitor peptides.

### Embodiment 195

The method of Embodiment 175, wherein the first predetermined threshold is a binding signal in the presence of competitor peptides of at least 5% of the binding signal as compared in the absence of competitor.

### Embodiment 196

The method of Embodiment 175, wherein the second predetermined threshold is a binding signal in the presence of competitor peptides of at least 5% of the binding signal as compared in the absence of competitor.

### Embodiment 197

The method of Embodiment 175, wherein the antibody binding region(s) is a linear epitope of the target protein.

### Embodiment 198

The method of Embodiment 175, wherein the antibody binding regions(s) is a structural epitope of the target region.

### Embodiment 199

The method of Embodiment 198, wherein steps (b) through d in claim 213 are repeated with additional peptides chosen from the at least one of the individual peptides in step (a) of Embodiment 175.

### Embodiment 200

In some embodiments, disclosed herein are methods for identifying a target protein of an antibody, the method comprising:
(a) contacting a first peptide array with said antibody at one or more concentrations in the presence and absence of a plurality of competitor peptides at one or more concentrations to obtain one or more input amino acid sequences, wherein the identified input amino acid sequences exhibit a binding signal in the presence of the plurality of competitor peptides within a first predetermined threshold of the binding signal in the absence of the plurality of competitor peptides;
(b) obtaining one or more secondary peptide array(s) using one or more input amino acid sequences chosen from at least one of the individual peptides in step (a), a conserved motif derived from an alignment of the individuals peptides in step (a) or an aligned motif derived from an alignment of the individual peptides in step (a), the one or more secondary peptide arrays synthesized by:
   i. determining a number of synthesis steps;
   ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
   iii. assigning at least one monomer to each patterned mask; and
   iv. coupling the monomers onto the features, wherein (ii) and (iii) assembles one said synthesis step and said synthesis step is repeated to form the peptide array;
(c) contacting each of said secondary peptide array(s) with said antibody in the presence and absence of the plurality of competitor peptides to obtain a set of peptide sequences, wherein the identified set of peptide sequences exhibit a binding signal measured in the presence of the plurality of competitor peptides within a second predetermined threshold of the binding signal measured in the absence of the plurality of competitor peptides;
(d) aligning said set of peptide sequences with each other to obtain at least one predictive binding motif; and
(e) aligning said predictive binding motif as a search criteria against a protein database, thereby identifying target proteins of the antibody based on the protein database search results score.

### Embodiment 201

The method of Embodiment 200, wherein the competitor peptides comprise a biological sample.

### Embodiment 202

The method of Embodiment 200, wherein the biological sample is serum.

### Embodiment 203

The method of Embodiment 200, wherein the competitor peptides are derived from the target protein.

### Embodiment 205

The method of Embodiment 203, wherein the competitor peptides are at least 50% similar to the target protein.

### Embodiment 206

The method of Embodiment 200, wherein the competitor peptides are derived from a known epitope of the antibody.

### Embodiment 207

The method of Embodiment 206, wherein the competitor peptides are at least 50% similar to the known epitope of the antibody.

### Embodiment 208

The method of Embodiment 200, wherein the competitor peptides comprise a biological sample and a peptide of any of Embodiments 203 to 208.

### Embodiment 209

The method of Embodiment 200, wherein the peptide array comprises at least 1000, at least 10,000, at least 100,000 or at least 1,000,000 unique peptides.

### Embodiment 210

The method of Embodiment 200, wherein the peptide array is in situ synthesized.

### Embodiment 211

The method of Embodiment 200, wherein the first peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

### Embodiment 212

The method of Embodiment 200, wherein the binding signal is measured as an intensity of the signal in the absence and presence of the competitor peptides at one or more concentrations.

### Embodiment 213

The method of Embodiment 200, wherein an apparent Kd is obtained in the presence and absence of the competitor peptides at one or more concentrations.

### Embodiment 214

The method of Embodiment 200, wherein at least one additional antibody is contacted with the peptide array, and the alignment scores obtained with each antibody are ranked to determine the propensity of each antibody to bind to the protein target.

### Embodiment 215

The method of Embodiment 200, further comprising determining a metric score for each antibody, wherein each antibody is assigned a single binding profile metric derived from the combination of the alignment scores from step (b) in claim 241 and the signal of the individual peptides of step (a) with more than one aligned position from step (b).

### Embodiment 216

The method of Embodiment 200, further comprising determining a metric score for each antibody, wherein each antibody is assigned a single specificity profile metric derived from the combination of the alignment scores from step (b) in claim 241, the number of peptides with more than one aligned position from step (b) and the signal of the individual peptides of step (a) with more than one aligned position from step (b).

### Embodiment 217

The method of Embodiment 200, further comprising aligning the at least one antibody epitope as a search criteria against a protein database.

### Embodiment 218

The method of Embodiment 217, wherein the protein database is a proteome database and wherein additional antibody target proteins and/or cross-reactive proteins are identified

### Embodiment 219

The method of Embodiment 200, wherein the first predetermined threshold is a binding signal in the presence of competitor peptides within at least 20-fold of the binding signal in the absence of competitor peptides.

### Embodiment 220

The method of Embodiment 200, wherein the predetermined threshold is a binding signal in the presence of competitor peptides of at least 5% of the binding signal as compared in the absence of competitor.

### Embodiment 221

In some embodiments, disclosed herein are methods for determining the propensity of antibody binding to at least one protein target, the method comprising:
(a) contacting a peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the individual peptides of step (a) to a first protein target, wherein the alignments between the individual peptides of step (a) and the first protein target are assigned alignment scores;
(c) repeating the alignment of individual peptides of step (a) with at least one additional protein target(s), wherein the alignments between the individual peptides of step (a) and the additional protein targets are assigned alignment scores; and
(d) comparing the alignment scores from steps (b) and (c) to obtain a relative propensity of the antibody to bind to said protein targets.

### Embodiment 222

The method of Embodiment 221, wherein the competitor peptides comprise a biological sample

### Embodiment 223

The method of Embodiment 222, wherein the biological sample is serum.

### Embodiment 224

The method of Embodiment 221, wherein the competitor peptides are derived from the target protein.

### Embodiment 225

The method of Embodiment 221, wherein the competitor peptides are at least 50% similar to the target protein.

### Embodiment 226

The method of Embodiment 225, wherein the competitor peptides are derived from a known epitope of the antibody.

### Embodiment 227

The method of Embodiment 221, wherein the competitor peptides comprise a biological sample and a peptide of any of Embodiments 224 to 226.

### Embodiment 228

The method of Embodiment 221, wherein the peptide array comprises at least 1000, at least 10,000, at least 100,000 or at least 1,000,000 unique peptides.

### Embodiment 229

The method of Embodiment 221, wherein the peptide array is in situ synthesized.

### Embodiment 230

The method of Embodiment 221, wherein the peptide array is synthesized by:
i. determining a number of synthesis steps;
ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (b) and (c) assembles one said synthesis step and said synthesis step is repeated to form the peptide array.

### Embodiment 231

The method of Embodiment 221, wherein the binding signal is measured as an intensity of the signal in the absence and presence of the competitor peptides at one or more concentrations.

### Embodiment 232

The method of Embodiment 221, wherein an apparent Kd is obtained in the presence and absence of the competitor peptides at one or more concentrations.

### Embodiment 233

The method of Embodiment 221, wherein at least one additional antibody is contacted with the peptide array, and the alignment scores obtained with each antibody are ranked to determine the propensity of each antibody to bind to the protein target.

### Embodiment 234

The method of Embodiment 221, further comprising determining a metric score for each antibody, wherein each antibody is assigned a single binding profile metric derived from the combination of the alignment scores from step (b) in claim 264 and the signal of the individual peptides of step (a) with more than one aligned position from step (b).

### Embodiment 235

The method of Embodiment 221, further comprising determining a metric score for each antibody, wherein each antibody is assigned a single specificity profile metric derived from the combination of the alignment scores from step (b) in claim 264, the number of peptides with more than one aligned position from step (b) and the signal of the individual peptides of step (a) with more than one aligned position from step (b).

### Embodiment 236

The method of Embodiment 221, further comprising aligning the at least one antibody epitope as a search criteria against a protein database.

### Embodiment 237

The method of Embodiment 236, wherein the protein database is a proteome database and wherein additional antibody target proteins and/or cross-reactive proteins are identified.

### Embodiment 238

The method of Embodiment 221, wherein the predetermined threshold is a binding signal in the presence of competitor peptides within at least 20-fold of the binding signal in the absence of competitor peptides.

### Embodiment 239

The method of Embodiment 221, wherein the predetermined threshold is a binding signal in the presence of competitor peptides of at least 5% of the binding signal as compared in the absence of competitor

### Embodiment 240

In some embodiments, methods are disclosed herein for determining the propensity of antibody binding to at least one protein target, the method comprising:
(a) contacting a first peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the one or more individual peptides of step (a) to obtain at least one predictive target motif;
(c) aligning the at least one predictive target motif to a first protein target, wherein the alignments between the individual peptides of step (a) and the first protein target are assigned alignment scores;
(d) repeating the alignment of at least one predictive target motif of step (b) with at least one additional protein target(s), wherein the alignments between the at least one predictive target motif of step (b) and the additional protein target(s) are assigned alignment scores; and
(e) comparing the alignment scores from steps (c) and (d) to obtain a relative propensity of the antibody to bind to said protein targets.

### Embodiment 241

The method of Embodiment 240, wherein the competitor peptides comprise a biological sample.

### Embodiment 242

The method of Embodiment 240, wherein the biological sample is serum.

### Embodiment 243

The method of Embodiment 240, wherein the competitor peptides are derived from the target protein.

### Embodiment 244

The method of Embodiment 243, wherein the competitor peptides are at least 50% similar to the target protein.

### Embodiment 245

The method of Embodiment 240, wherein the competitor peptides are derived from a known epitope of the antibody.

### Embodiment 246

The method of Embodiment 245, wherein the competitor peptides are at least 50% similar to the known epitope of the antibody.

### Embodiment 247

The method of Embodiment 240, wherein the competitor peptides comprise a biological sample and a peptide of any of Embodiments 243 to 246.

### Embodiment 248

The method of Embodiment 240, wherein the peptide array comprises at least 1000, at least 10,000, at least 100,00 or at least 1,000,000 unique peptides.

### Embodiment 249

The method of Embodiment 240, wherein the peptide array is in situ synthesized.

### Embodiment 250

The method of Embodiment 240, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

### Embodiment 251

The method of Embodiment 240, wherein the binding signal is measured as an intensity of the signal in the absence and presence of the competitor peptides at one or more concentrations.

### Embodiment 252

The method of Embodiment 240, wherein an apparent Kd is obtained in the presence and absence of the competitor peptides at one or more concentrations.

### Embodiment 253

The method of Embodiment 240, wherein at least one additional antibody is contacted with the peptide array, and the alignment scores obtained with each antibody are ranked to determine the propensity of each antibody to bind to the protein target.

### Embodiment 254

The method of Embodiment 240, further comprising determining a metric score for each antibody, wherein each antibody is assigned a single binding profile metric derived from the combination of the alignment scores from step (b) in claim 287 and the signal of the individual peptides of step (a) with more than one aligned position from step (b).

### Embodiment 255

The method of Embodiment 240, further comprising determining a metric score for each antibody, wherein each antibody is assigned a single specificity profile metric derived from the combination of the alignment scores from step (b) in claim 287, the number of peptides with more than one aligned position from step (b) and the signal of the individual peptides of step (a) with more than one aligned position from step (b).

### Embodiment 256

The method of Embodiment 255, further comprising aligning the at least one antibody epitope as a search criteria against a protein database.

### Embodiment 257

The method of Embodiment 240, wherein the protein database is a proteome database and wherein additional antibody target proteins and/or cross-reactive proteins are identified.

### Embodiment 258

The method of Embodiment 240, wherein the predetermined threshold is a binding signal in the presence of competitor peptides within at least 20-fold of the binding signal in the absence of competitor peptides.

### Embodiment 259

The method of Embodiment 240, wherein the predetermined threshold is a binding signal in the presence of competitor peptides of at least 5% of the binding signal as compared in the absence of competitor

### Embodiment 260

In some embodiments disclosed herein are kits and systems for characterizing antibody binding against at least one protein target, the kits and systems comprising:
(a) providing a peptide array,
(b) providing a plurality of competitor peptides
(c) providing instructions for a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides;
(d) providing instructions for the user to align the individual peptides to said at least one protein target, wherein the alignments between the individual peptides of step (c) and at least one protein target are assigned alignment scores; and
(e) providing instructions for the user to characterize binding of the antibody against the at least one protein target using the alignment scores of step (d).

### Embodiment 261

In some embodiments disclosed herein are kits and systems for identifying an antibody epitope in a target protein, the kits and systems comprising:
(a) providing a peptide array;
(b) providing a plurality of competitor peptides;
(c) providing instructions for a user to contact the peptide array with said antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions for the user to align the individual peptides to said at least one protein target, wherein the alignments between the individual peptides of step (c) and at least one protein target are assigned alignment scores; and
(e) providing instructions for the user to determine conserved amino acids in the individual peptides of step (c) to identify a conserved binding peptide motif and aligning the individual motifs to said at least one target protein in order to identify at least one antibody epitope of the target protein.

### Embodiment 262

In some embodiments disclosed herein are kits and systems for for characterizing antibody binding regions in a target protein, the kits and systems comprising:
(a) providing a first peptide array;
(b) providing a plurality of competitor peptides;
(c) providing instructions for a user to contact a first peptide array with an antibody in the presence and absence of the plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides;
(d) providing instructions for a user to create a second peptide array using an input peptide sequence chosen from at least one of the individual peptides in step (c), a conserved motif derived from an alignment of the individuals peptides in step (c) or an aligned motif derived from an alignment of the individual peptides in step (c), the second peptide array synthesized by:
   i. determining a number of synthesis steps;
   ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (ii) and (iii) assembles one said synthesis step and said synthesis step is repeated to form the peptide array;
(e) providing instructions for the user to contact the second peptide array with the antibody to identify a second set of peptides; and
(f) providing instructions for the user to contact the second peptide array with said antibody in the presence of the plurality of competitor peptides, and identifying a second set of individual peptides from step (e) that exhibit a binding signal within a second predetermined threshold of the binding signal in step (e); and
(g) providing instructions for a user to align said second set of individual peptides to said target protein and identifying regions in the target protein which align to the second set of individual peptides identified, thereby characterizing antibody binding regions in the target protein.

### Embodiment 263

In some embodiments disclosed herein are kits and systems for for determining the propensity of antibody binding to at least one protein target, the kits and systems comprising:
(a) providing a peptide array;
(b) providing a plurality of competitor peptides
(c) providing instructions to a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions to the user to align the individual peptides of step (c) to a first protein target, wherein the alignments between the individual peptides of step (c) and the first protein target are assigned alignment scores;
(e) providing instructions to the user to repeat the alignment of individual peptides of step (c) with at least one additional protein target(s), wherein the alignments between the individual peptides of step (c) and the additional protein targets are assigned alignment scores; and
(f) providing instructions to the user to compare the alignment scores from steps (c) and (d) to obtain a relative propensity of the antibody to bind to said protein targets.

### Embodiment 264

In some embodiments disclosed herein are kits and systems for determining the propensity of antibody binding to at least one protein target, the kits and systems comprising:
(a) providing a first peptide array;
(b) providing a plurality of competitor peptides;
(c) providing instructions for a user to contact the first peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions for the user to align the one or more individual peptides of step (c) to obtain at least one predictive target motif;
(e) providing instructions for the user to align the at least one predictive target motif to a first protein target, wherein the alignments between the individual peptides of step (c) and the first protein target are assigned alignment scores;
(f) providing instructions for the user to repeat the alignment of at least one predictive target motif of step (e) with at least one additional protein target(s), wherein the alignments between the at least one predictive target motif of step (e) and the additional protein target(s) are assigned alignment scores; and
(g) providing instructions for the user to compare the alignment scores from steps (c) and (d) to obtain a relative propensity of the antibody to bind to said protein targets.

### EXAMPLES

### Example 1—in Silico Simulation

In some embodiments, masking algorithm is simulated by an in silico method. In this example, a simulation includes the following parameters:

Total number of features: 500,000

Percent Overlap between Mask n and Mask n−1: 52%

Input Sequence: HVGAAAPVVPQA (A Disease-Correlated Epitope)

Number of Synthesis Steps: 21

Synthesis Order of Addition: A,R,Q,S,P,W,V,V,P,A,D,A,A,M,G,V,F,H,K,L,Y
Synthesis order of addition is chosen by the user to generate a sequence space more or less closely related to the input sequence (see above), however, all amino acids in the input sequence must be included in the synthesis order of addition. Synthesis orders of coupling similar to the order of amino acids in the input sequence will generate a space more closely related to the input sequence; conversely, synthesis orders of coupling that are less similar to the order of amino acids in the input sequence will generate sequence spaces less closely related to the input sequence.

A pseudo code of the simulation is described below:

```
librarySize <- 500000
fractionSeqMaskOverlap <- 0.52
inputSequence <- “H V G A A A P V V P Q A ”
synthesisOrder <- c(“A”, “R”, “Q”, “S”, “P”, “W”, “V”, “V”, “P”,
“A”, “D”, “A”, “A”, “M”, “G”, “V”, “F”, “H”, “K”, “L”, “Y”)
numSteps <- length(synthesisOrder)
numFeaturePerMask <- floor(fractionSeqMaskOverlap*librarySize)
librarySeqs <- rep(‘’, librarySize)
libraryLengths <- rep(0, librarySize)
inputSeqReps <- c( )
n_1Mask <- rep(FALSE, librarySize)
n_1Mask[sample(seq(1:librarySize), numFeaturePerMask)] <- TRUE
for(currStep in synthesisOrder) {
  str(currStep)
  n_1MaskOpen <- which(n_1Mask, arr.ind = TRUE)
  for(currIdx in n_1MaskOpen){
    librarySeqs[currIdx] <- paste(currStep, librarySeqs[currIdx])
    libraryLengths[currIdx] <- length(librarySeqs[currIdx])/2
    if(librarySeqs[currIdx] == inputSequence) {
      inputSeqReps <- append(inputSeqReps, currIdx)
    }
    }
  ; <- sort(sample(n_1MaskOpen,floor(fractionSeqMaskOverlap*
                            length(n_1MaskOpen))))
    diffIndices <- setdiff(1:librarySize, n_1MaskOpen)
    nMaskOpen <- sort(sample(diffIndices, numFeaturePerMask-
                      length(overlapIndices)))
    nMaskOpenIndices <- sort(append(overlapIndices,nMaskOpen))
    nMask <- rep(FALSE, librarySize)
    nMask[nMaskOpenIndices] <- TRUE
    n_1Mask <- nMask
  }
```

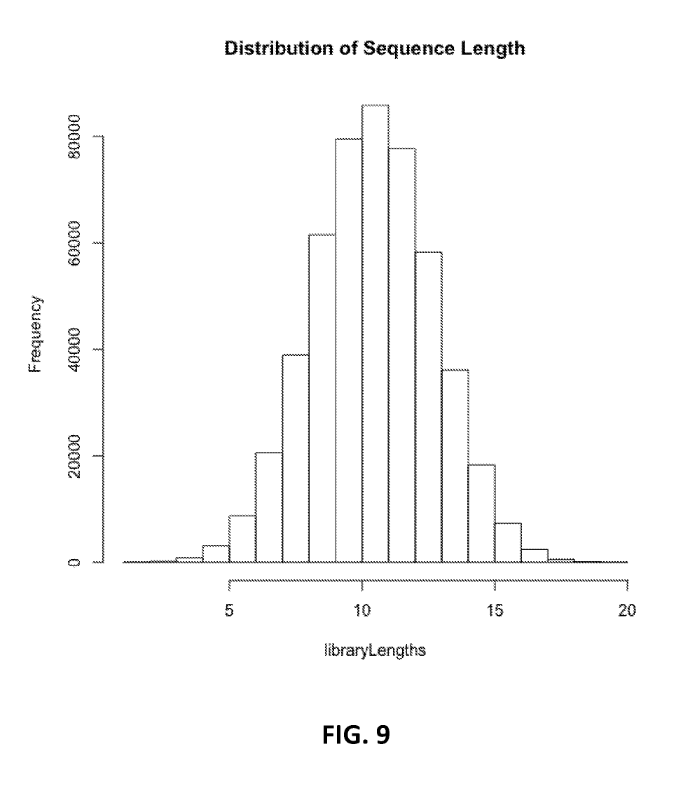

Consider the following input and parameter setting: the input sequence is HVGAAAPVVPQA; the number of synthesis steps is 21; library size includes 500,000 features; the number of input sequence replicates in sampled space is 3; an average number of replicates for all sequences in sampled space is 1.4 reps/sequence; the number of distinct sequences in sampled space is 360,064 sequences. FIG. 9 shows distribution of sequence length in simulated library generated using the mask and synthesis algorithm disclosed herein, wherein the median length is 11.

Further, the following table shows an example set of sequences selected from the sequence space generated using a simulation of the mask and synthesis algorithm disclosed herein.

| | | |
|---|---|---|
| H M A D V V S Q R A | L H V G A A A P V V P Q A | H V G M D P V W Q A |
| G A A P P Q A | H V P V V W P Q A | K F V M A A A V S A |
| L F V G M P V | Y L H F V M D V V Q | M A D A P V W P S Q A |
| Y K G M A D V W P S Q A | Y L H F Q R A | Y V G A P V W S |
| H F V G M A A A V P S Q R | K V M A D A V V P Q A | K H A A P W P S |
| K H F V M A A P V W P A | Y K H V M A D P V Q R | Y L K F M A D V V W P S R A |
| L K H V G A A A P V V P Q A | L H F V A A P V P S R | L K H V G M D P V V P S |
| Y L F V G A D P W S R A | L F M A A P Q A | L K H F G A A A V V P Q |
| F V A D A P V P S Q R A | K H F G V W Q R A | K H V A A D A V W P A |
| Y L K H V A A A W P S R A | Y L H V M A A A V P A | Y L H V G A A A P V V P Q A |
| Y H F V G M A A A V A | G M A D A P V P Q R | Y H V G A A P V W A |

### Example 2: Characterization of Antibody Binding Profiles on High-Density Peptide Arrays

Identification of Anti HER2 mAb Binding to Array Peptides

A competition binding assay was designed to identify array peptides that reflect the biological binding of mAbs. Array peptides having this characteristic were identified as individual peptides. A ranking of the individual peptides could be applied, where significant peptides are defined as more than one exact match without gaps, although peptides with matches of varying degrees with gaps were also acceptable. The assay was performed to identify significant peptides in 14 commercially available therapeutic or research monoclonal antibodies to HER2 (Table 1). The panel included mAbs from different clones raised to different immunogens, different clones raised to the same immunogen, and identical clones obtained from different vendors. The apparent Kd (concentration of antibody at half-maximal saturable binding) was measured for each mAb in the absence and the presence competitors as described below.

TABLE 1
Anti-HER2 panel of monoclonal antibodies

| | | HER2 Immunogen | | | |
|---|---|---|---|---|---|
| Antibody Name & Clone | Host | Sequence Region | Clonality | Part# | Supplier |
| Neu Antibody (C-12): sc-374382 | murine | 983-1017 | mono | sc-374382 | SCBT |
| Neu Antibody (C-3): sc-377344 | murine | 251-450 | mono | sc-377344 | SCBT |
| Neu Antibody (A-2): sc-393712 | murine | 1180-1197 | mono | sc-393712 | SCBT |
| Neu Antibody (3B5) | murine | 1242-1255 | mono | sc-33684 | SCBT |
| Monoclonal Anti-HER2 antibody 4B8 | murine | 22-122 | mono | WH0002064M6 | Sigma |
| Monoclonal Anti-HER2 antibody CL0268 | murine | 274-400 | mono | AMAB90627 | Sigma |
| Anti-ERBB2/HER2 Antibody (aa676-1255, | murine | 676-1255 | mono | LS-C337488 | LSBio |
| clone 11A7) | | | | | |
| Anti-ERBB2/HER2 Antibody (aa23-652) | murine | 23-652 | mono | LS-C128811 | LSBio |
| HER-2/ErbB2 Antibody (6C2) | murine | 750-987 | mono | MA5-15702 | Thermo |
| HER-2/ErbB2 Antibody (3B5) | murine | C-terminus | mono | MA5-13675 | Thermo |
| HER2/ErbB2 (D8F12) XP ® Rabbit mAb | rabbit | N-terminus | mono | #4290 | Cell Signaling |
| HER2/ErbB2 (44E7) Mouse mAb | murine | C-terminus | mono | #2248 | Cell Signaling |
| HER2/ErbB2 (29D8) Rabbit mAb | rabbit | 1242-1255 | mono | #2165 | Cell Signaling |
| Anti-Human ErbB2 Therapeutic Antibody | Recomb | human ERBB2 | mono | TAB-005 | Creative Biolabs |
| (trastuzumab) | Humanized | | | | |

Competitive Binding Assay.

Microarrays comprising diverse peptide arrays or focused peptide libraries (described in Example 1) were obtained and rehydrated prior to use by soaking with gentle agitation in distilled water for 1 h, PBS for 30 min and primary incubation buffer (PBST, 1% mannitol) for 1 h. Slides comprising the microarrays were loaded into an ArrayIt microarray cassette (ArrayIt, Sunnyvale, Calif.) to adapt the individual microarrays to a microtiter plate footprint. mAb solutions of six different concentrations of each mAb: 3 nM, 1 nM, 0.33 nm, 0.11 nM, 0.0367 nM, and 0.012 nM, were prepared by serially diluting the stock in incubation buffer (PBST, 1% mannitol).

mAb binding was assayed in the absence or presence of two different concentrations of serum competitor (1/69 ND, and 1/71ND) or in the absence or presence of two different concentrations of a mixture of competitor peptides (250 μM, and 750 μM). The mixture of competitor peptides consisted of 24 peptides chosen according to the following criteria: a) for providing a mixture having a balanced amino acid composition i.e. peptides that were not enriched for any one amino acid; b) for having a GRAVY score <0 to ensure solubility in an aqueous assay; and c) for having a balanced and continuous range of isoelectric points (pI) ranging from pI=3 to pI=10. The arrays were incubated with the different mAbs solutions for 30 minutes at 37° C. with mixing on a TeleShake95 (INHECO, Martinsried, Germany) to allow for antibody-peptide binding. Following incubation, the cassette was washed in PBST (PBS-Tween) at 10× chamber volumes. Depending upon the origin of the primary antibody, bound mAb was detected using either 4.0 nM goat anti-human IgG (H+L), goat anti-rabbit, or goat anti-mouse secondary antibodies conjugated to AlexaFluor 647 (Thermo-Invitrogen, Carlsbad, Calif.). Binding of the secondary antibody was allowed to proceed in incubation buffer (3% BSA in PBST) for 1 hour at 37° C. while mixing on a TeleShake95 platform mixer. Following incubation with secondary antibody, the slides were again washed with PBST at 10× chamber volumes and distilled water, removed from the cassette, sprayed with isopropanol and centrifuged dry.

Data Acquisition.

Assayed library arrays were imaged using an Innopsys 910AL microarray scanner fitted with a 532 nm laser and 572 nm BP 34 filter (Innopsys, Carbonne, France). The Mapix software application (version 7.2.1) identified regions of the images associated with each peptide feature using an automated gridding algorithm. Median pixel intensities for each peptide feature were saved as a tab-delimitated text file and stored in a database for analysis. Quantitative signal measurements were obtained at a 1 μM resolution and 1% feature saturation by determining a relative fluorescent value for each addressable peptide feature. Thirty measurements of binding were obtained for each of the mAbs that were assayed.

Signal Analysis.

Binding of mAbs to each feature was measured by quantifying fluorescent signal. The median feature intensities were first background subtracted relative to the negative controls (secondary antibody only), then log₁₀ transformed, then normalized by dividing by the log₁₀ transformed median.

Specificity of Array Peptide Binding.

Specificity herein refers to the degree to which an antibody differentiates two different antigens. (Ref: Immunology and Infectious Disease, S. A. Frank, 2002, Princeton Univ. Press). Binding specificity for each array peptide was characterized by the difference in binding signal obtained in the absence and in the presence of competitor, and the degree to which binding was attenuated by non-cognate peptide competitors or serum competitor provided a measure of mAb specificity. Peptide binding specificity was determined by the difference in the apparent Kd value for each array peptide in the absence of competitor and in the presence of each of serum and non-cognate peptide competitor.

Results.

The data showed that array peptides exhibit saturable mAbs dose-response binding in the absence of competitor, and that saturable binding was maintained in the presence of serum or of peptide competitor. Subsequently, the decrease in apparent Kd, determined in the presence of competitor relative to that obtained in the absence of competitor was used to select individual peptides, in this case significant peptides, from the peptide library array screen.

TABLE 2
Herceptin Apparent Kd Results

| | | Apparent Kd 750 |
|---|---|---|
| Herceptin Binding | Apparent Kd | uM Peptide Mix |
| Peptide Sequence | No Competitor | Competitor |
| FGPYKPFGAQ | 0.001 | 0.010 |
| PYKFFP | 0.001 | 0.003 |
| EYKPFWKGAP | 0.001 | 0.010 |
| FGPQYKPFQP | 0.001 | 0.001 |
| FGPYKPIGAQPP | 0.002 | 0.007 |
| FGEQYKPPIWKGAQPP | 0.003 | 0.008 |
| FGPQYKPI | 0.003 | 0.020 |
| QPFWKFQP | 0.005 | 0.010 |
| QPFPIWKGAQP | 0.010 | 0.020 |
| FGPQYKPIWKFQP | 0.020 | 0.020 |

Peptides were ranked according to the fold-change in apparent Kd. Individual peptides, including significant peptides, were selected for having a change in apparent Kd when measured in the presence of competitor that was less than a 10-fold decrease in Kd when measured in the absence of each competitor.

Subsequently, the individual peptides, including significant peptides, were used to identify linear and structural epitopes, identify key amino acids within the target epitopes, to determine the binding specificity of the mAbs, and to identify unknown protein targets.

### Example 3: Diverse and Focused Peptide Arrays

Diverse Libraries.

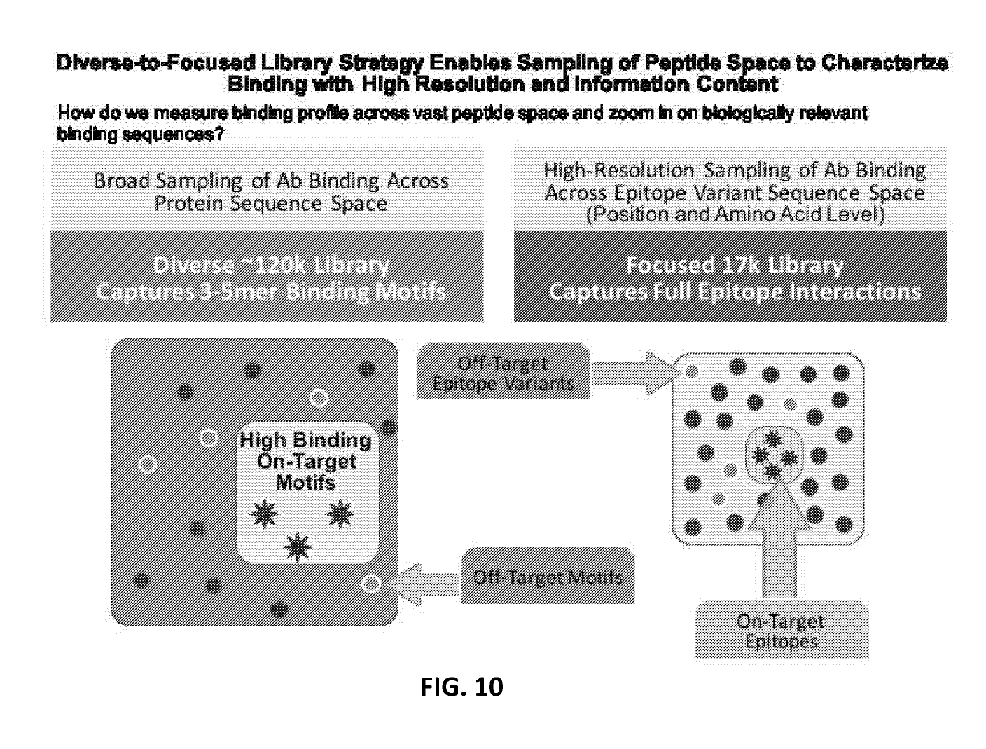

Diverse peptide libraries were prepared to sample the highly diverse sequence space represented in a combinatorial peptide library, and provide individual peptides, including significant peptides, comprising enriched in motifs that predicted biding epitopes. The enriched motifs served as basis for identifying input sequences that were used to design focused libraries. See FIG. 10.

The diverse library used in the methods provided was prepared as a primary highly diverse combinatorial library of 126,009 peptides with a median length of 9 residues, ranging from 5 to 13 amino acids, and designed to include 99.9% of all possible 4-mers and 48.3% of all possible 5-mers of 16 amino acids (methionine, M; cysteine, C; isoleucine, I; and threonine, T were excluded). The peptides were synthesized on an 200 mm silicon oxide wafer using standard semiconductor photolithography tools adapted for tert-butyloxycarbonyl (BOC) protecting group peptide chemistry (Legutki J B et al., Nature Communications. 2014; 5:4785). Briefly, an aminosilane functionalized wafer was coated with BOC-glycine. Next, photoresist containing a photoacid generator, which is activated by UV light, was applied to the wafer by spin coating. Exposure of the wafer to UV light (365 nm) through a photomask allows for the fixed selection of which features on the wafer will be exposed using a given mask. After exposure to UV light, the wafer was heated, allowing for BOC-deprotection of the exposed features. Subsequent washing, followed the by application of an activated amino acids completes the cycle. With each cycle, a specific amino acid was added to the N-terminus of peptides located at specific locations on the array. These cycles were repeated, varying the mask and amino acids coupled, to achieve the combinatorial peptide library. Thirteen rectangular regions with the dimensions of standard microscope slides, were diced from each wafer. Each completed wafer was diced into 13 rectangular regions with the dimensions of standard microscope slides (25 mm×75 mm). Each of these slides contained 24 arrays in eight rows by three columns. Finally, protecting groups on the side chains of some amino acids were removed using a standard cocktail. The finished slides were stored in a dry nitrogen environment until needed. A number of quality tests are performed ensure arrays are manufactured within process specifications including the use of 3σ statistical limits for each step. Wafer batches were sampled intermittently by MALDI-MS to identify that each amino acid was coupled at the correct step, ensuring that the individual steps constituting the combinatorial synthesis were correct. Wafer manufacturing was tracked from beginning to end via an electronic custom Relational Database which is written in Visual Basic and has an access front end with an SQL back end. The front-end user interface allows operators to enter production info into the database with ease. The SQL back end provides a simple method for database backup and integration with other computer systems for data share as needed. Data typically tracked include chemicals, recipes, time and technician performing tasks. After a wafer is produced the data is reviewed and the records are locked and stored. Finally, each lot is evaluated in a binding assay to confirm performance, as described below.

Monoclonal binding to the array peptides of the diverse library identified individual peptides, including significant peptides, that comprised 3-5 mer motifs, which were used to identify input sequences for designing focused libraries (FIG. 10).

Focused Libraries.

Focused libraries were prepared to vary a number of positions around the input sequence comprising enriched motifs of individual peptides, including significant peptides, identified in the diverse library. The focused library used in the methods provided was prepared as a library of 16,920 peptides using a series of 24 overlapping masks, which resulted in synthesized peptides with a median length of 0 to 17 amino acid residues.

The peptides of the focused library were designed each to provide variant sequences of one input sequence of an individual peptide, in this case a significant peptide, of the diverse library. The dimensions of each feature were 44 μm×44 μm, set at 50 μm×50 μm pitch, having a 6 μm interstitial space between features. The peptides were synthesized on an 200 mm silicon oxide wafer using standard semiconductor photolithography tools adapted for tert-butyloxycarbonyl (BOC) protecting group peptide chemistry (Legutki J B et al., Nature Communications. 2014; 5:4785), as described for the synthesis of the diverse peptide library. Wafer batches were sampled intermittently by MALDI-MS to identify that each amino acid was coupled at the correct step, ensuring that the individual steps constituting the focused synthesis were correct.

### Example 4: Identification of Epitopes

Identification of Predicted Epitopes of HER2.

Competition binding assays as described in Example 2, were performed on either a diverse or focused peptide array/library (or both) using anti HER2 mAbs SCBT sc-33684, Thermo MA5-13675, Cell Signaling #2165 and Creative Biolabs TAB-005 to identify individual peptides, including significant peptides, and predicted epitope sequences.

Binding peptides were ranked according to their level of relative specificity for the mAb, and individual peptides were selected as having less than a 10-fold decrease in apparent Kd, as described in Example 2. Individual peptides, specifically significant peptides, were selected for predicting HER2 epitope sequences for each of the mAbs that were tested.

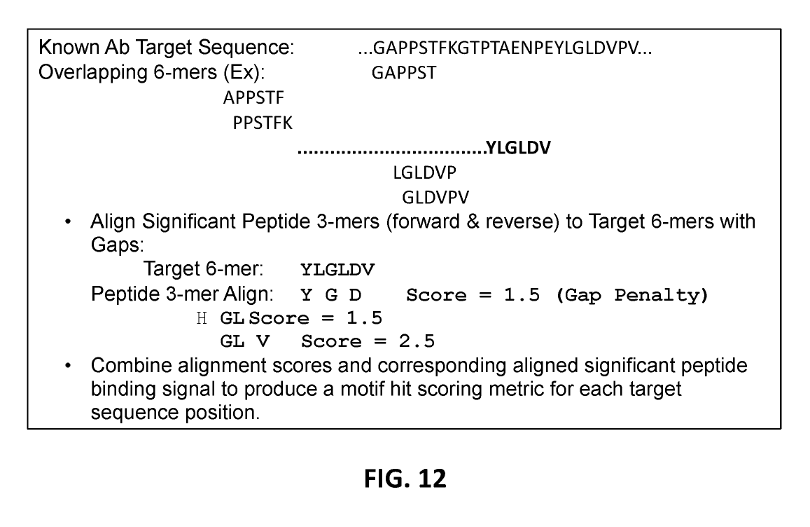

The array signal for each of the significant peptides was median normalized and log transformed, and significant peptides having a signal that was at least >2-fold above the median were aligned using ClustalW and MUSCLE alignments to overlapping 6-mer sequences of the HER2 protein (UNIPROT #P04626). Forward and reverse sequences of 3-mers of significant peptides were aligned to any of all possible HER2 target 6-mers, and a score for every amino acid position in the entire HER2 protein was determined. An alignment score was calculated as the sum of all scores at each position, and was combined with the binding signal of the corresponding significant peptide to provide a motif score (FIG. 12). The motif scores were sufficient to predict the target epitope.

Linear arrangement of submotifs was performed and compared using CLUSTALW (https://www.ncbi.nlm.nih.gov/pmc/articles/PMC308517/) and MUSCLE (https://www.ncbi.nlm.nih.gov/pmc/articles/PMC390337/) software.

The motifs were also ranked according to their enrichment in the significant peptides. Fold-enrichment was calculated relative to the incidence of the motif in all array peptides i.e. significant and non-significant library array peptides by determining the probability of a particular motif/probability of finding that motif randomly in the Library or Array. Table 3 shows an exemplary list of trimer motifs and the corresponding fold-enrichment.

Finally, significant peptides were aligned (CLUSTALW (https://www.ncbi.nlm.nih.gov/pmc/articles/PMC308517/) and MUSCLE (https://www.ncbi.nlm.nih.gov/pmc/articles/PMC390337/)) to determine the identity and position of conserved amino acids.

TABLE 3
Motifs Enriched in Significant Peptides

| | | | Enrichment |
|---|---|---|---|
| | | | False- |
| Enriched | Fold | Enrichment | Discovery |
| Motif | Enrichment | P-Value | Rate |
| EVE | 5.79 | 5.6699E-219 | 8.656E-217 |
| HEV | 8.13 | 5.3367E-206 | 4.8885E-204 |
| PWE | 5.88 | 3.7323E-144 | 1.3149E-142 |
| WEV | 7.65 | 1.0867E-119 | 3.1107E-118 |
| HEVG | 8.78 | 2.74964E-42 | 8.18782E-40 |

### Example 5: Identification of Linear Epitopes

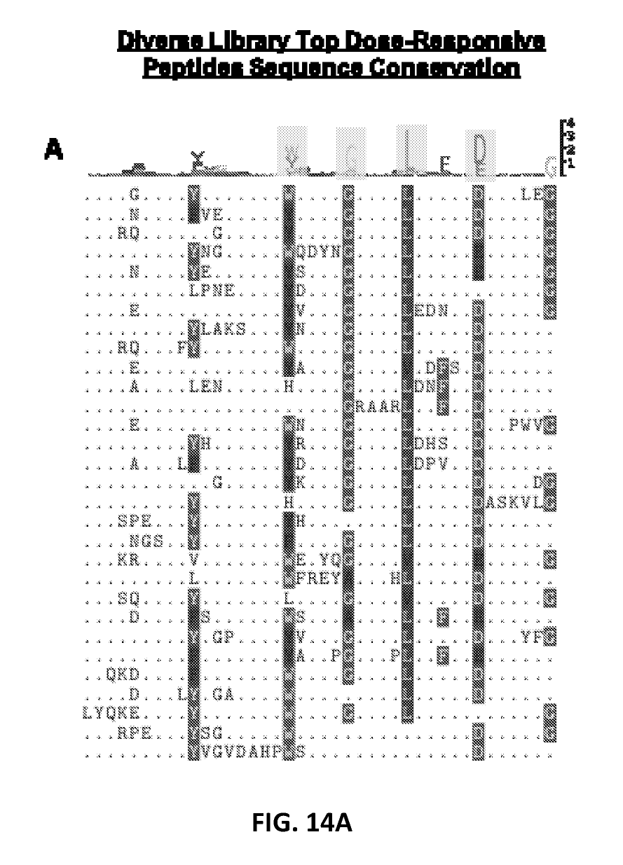

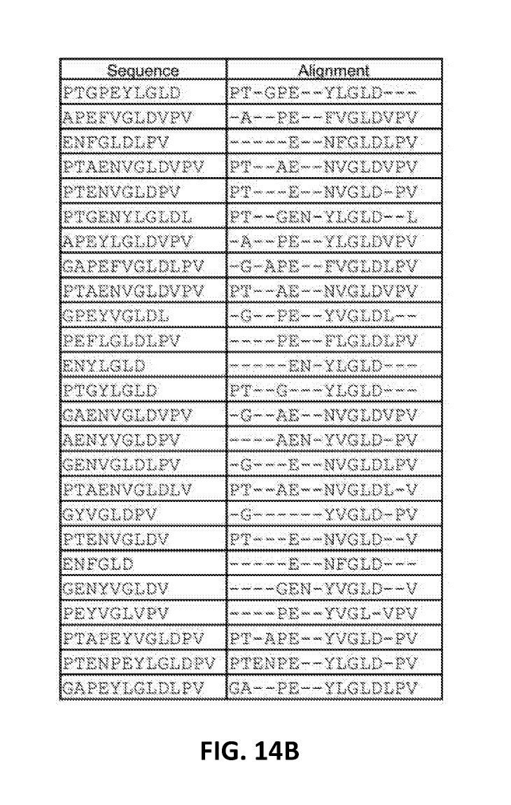

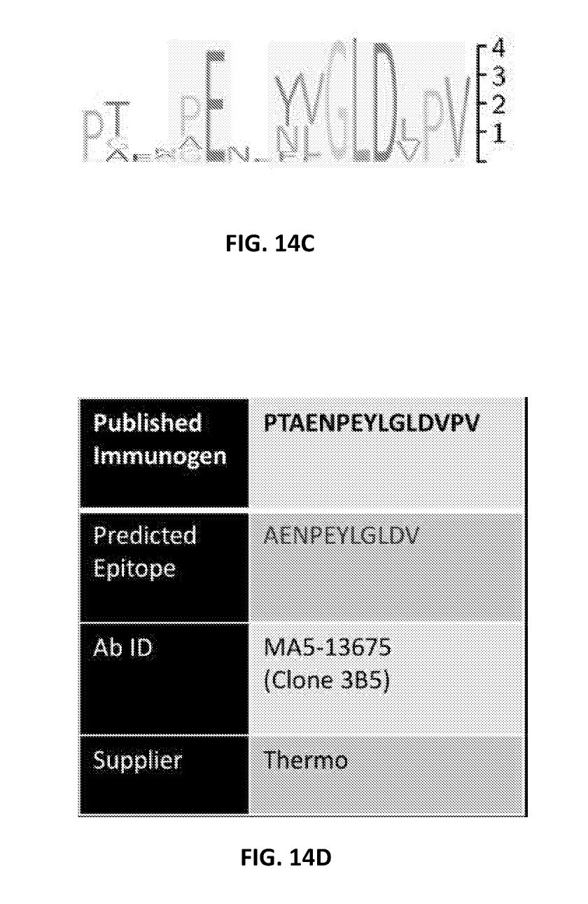

For each of the mAbs that were tested, individual peptides, including significant peptides were identified in the diverse library as described in Example 4. The corresponding enriched motifs were determined to predict HER2 epitopes, and conserved amino acids and their positions identified. The top dose-responsive peptide sequences identified from the diverse library of three exemplary anti-HER2 antibodies: MA5-13675 (clone 3B5) (Thermo Fisher; Waltham, Mass.), sc-33684 (clone 3B5) (Santa Cruz BioTechnologies, Dallas, Tex.), and 2165 (clone 29D8) (Cell Signalling Technologies, Danvers, Mass.) are shown in FIGS. 14, 15, and 16, respectively.

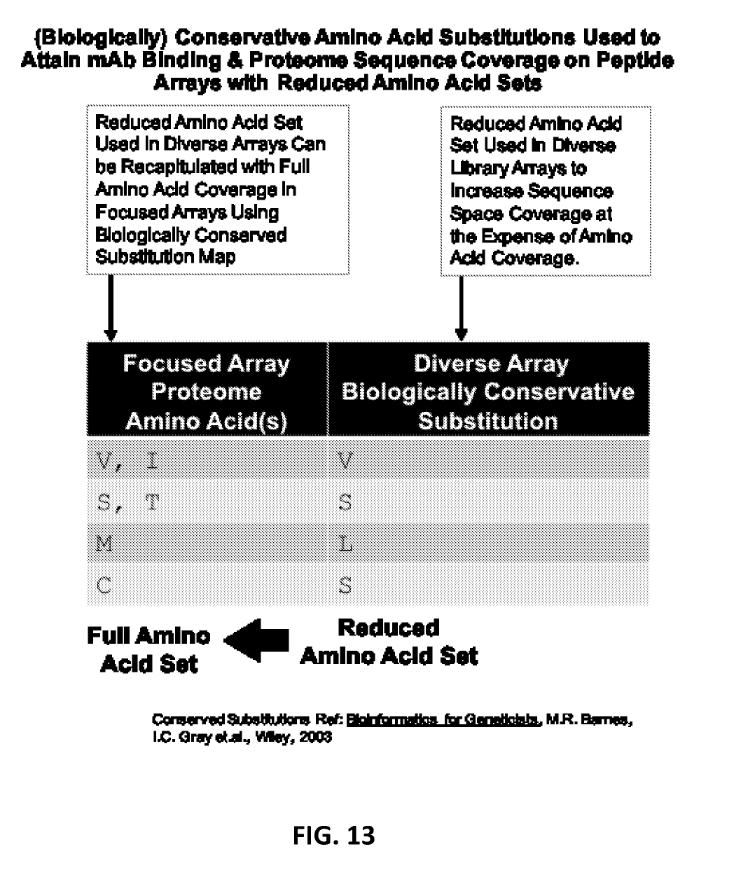

Enriched motifs were aligned against the HER2 protein to identify regions comprising the motifs that could be varied to design focused libraries. A reduced set of amino acids was used to map each residue of the motif to the protein target to reduce the number of amino acids that would be needed to sample the array while having full representation of the proteomic sequences to which epitopes can be mapped ((FIG. 13). Regions on the target protein comprising the trimer and tetramer motifs that were shown to be highly conserved across individual peptides, specifically significant peptides, identified in the diverse library, were used as input sequences to derive variant sequences thereof and comprising the conserved motifs for designing focused libraries.

Positional variants were generated through the process of developing the focused library algorithm as described in Example 2. These variants are derived from the input sequence, mask order and amino acid order defined during focused library design.

Individual peptides, in this case significant peptides, identified in each of the focused libraries were aligned to the HER2 target protein, scored according to their relative specificity, and aligned to identify the consensus sequences of the epitopes.

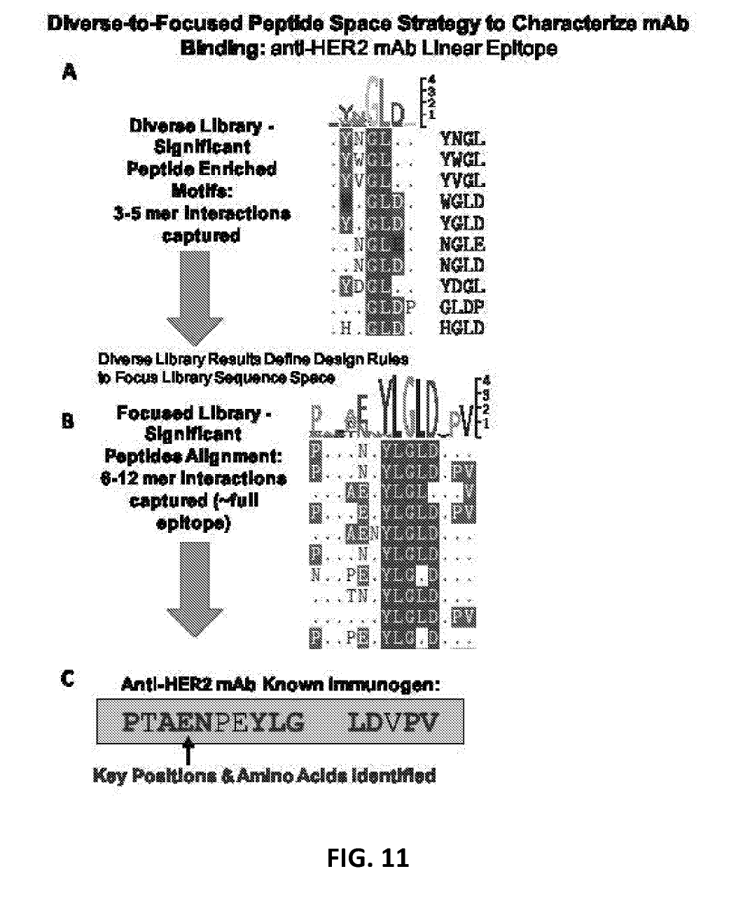

The alignments of the top significant peptides identified from the three focused libraries are shown in FIGS. 14B, 15B, and 16B. The positions of the conserved amino acids for mAb MA5-13675 (clone 3B5) (Thermo Fisher) (FIGS. 11B and 14C), show that one iteration of the combination screening of a diverse and a focus library, identified the full sequence of the linear HER2 epitope, which was encompassed in the immunogen (FIG. 14D).

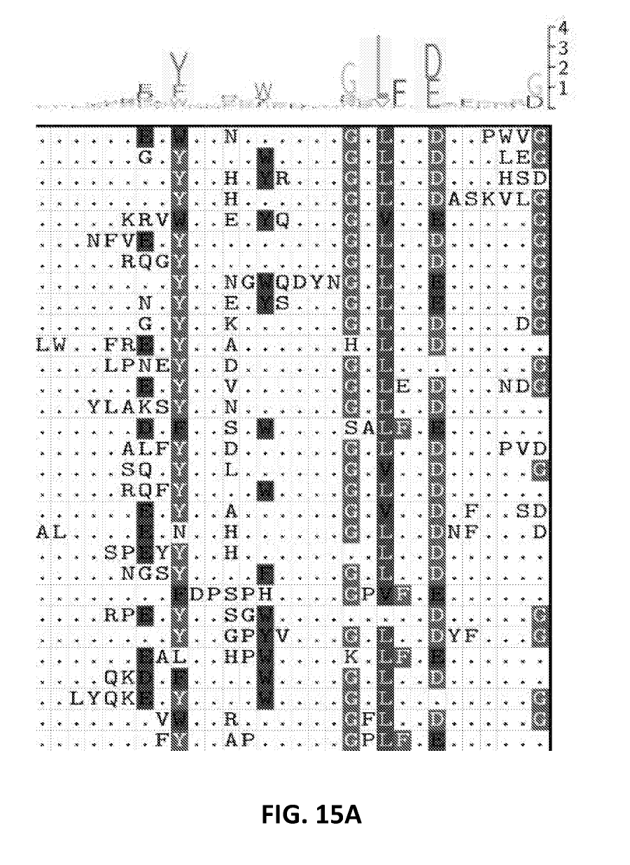

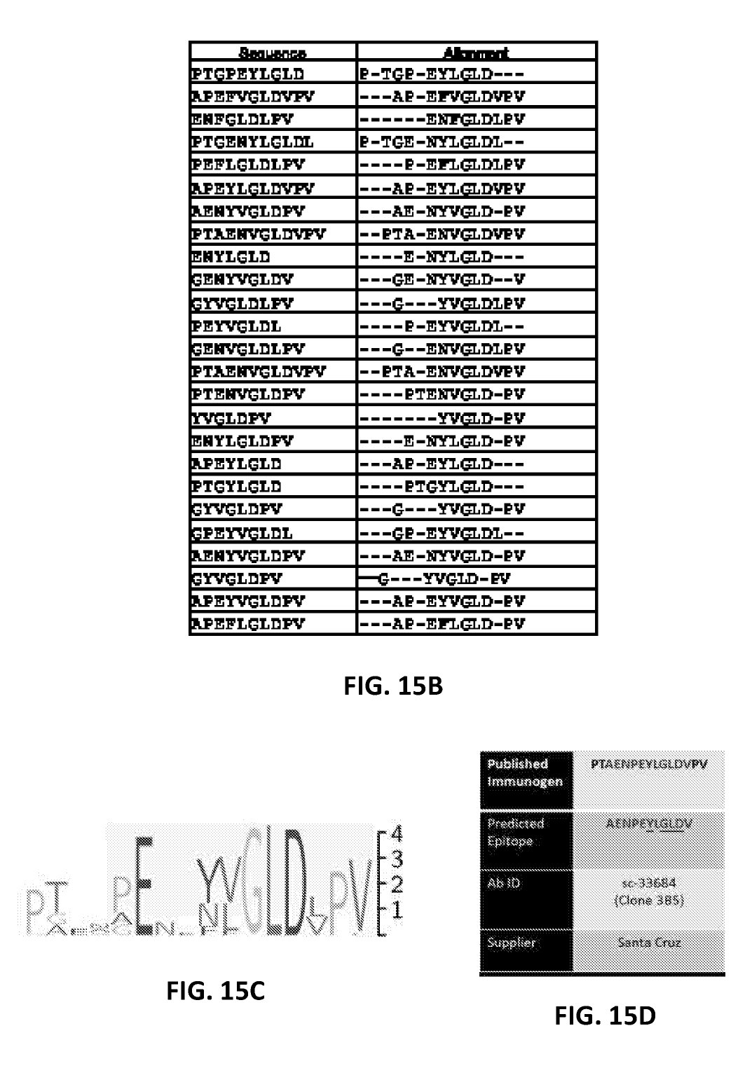

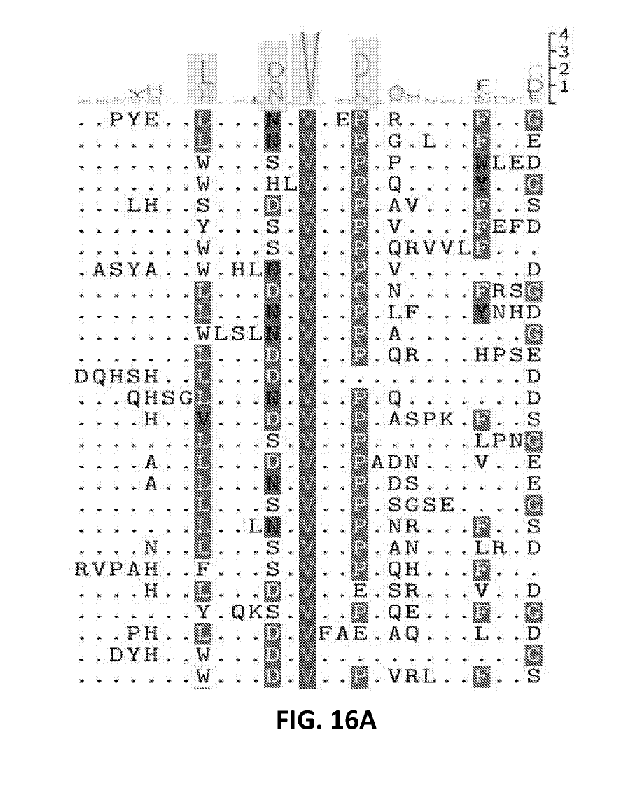

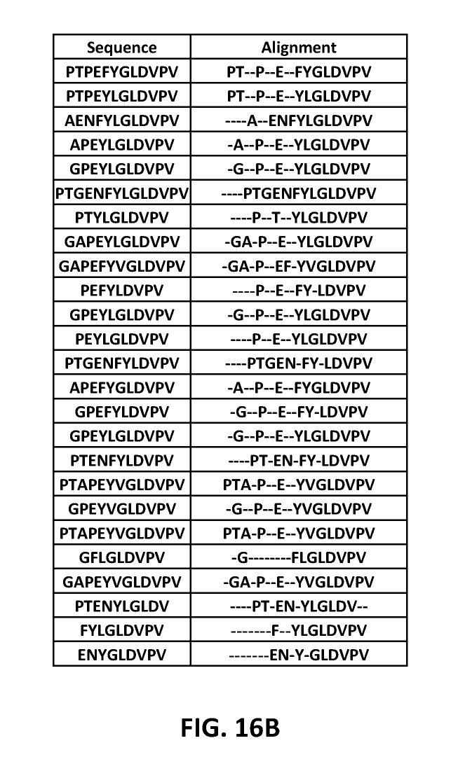

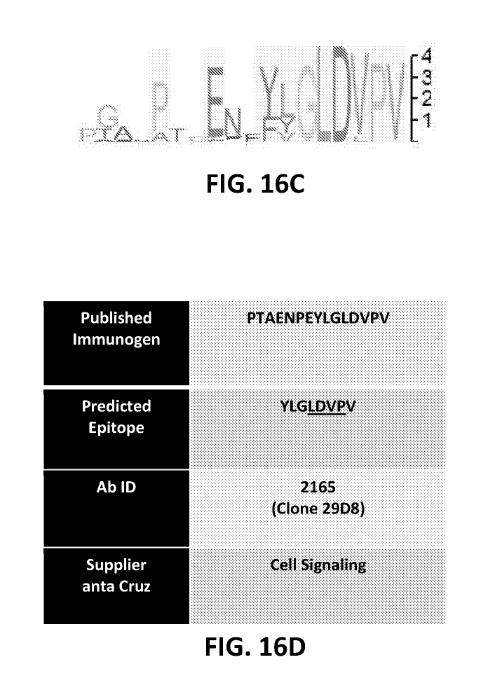

Similarly, the full linear epitope of anti HER monoclonal antibodies sc-33684 (clone 3B5) (Santa Cruz BioTechnologies), and 2165 (clone 29D8) (Cell Signalling Technologies) were correctly identified (FIGS. 15C and 15D, and FIGS. 16C and 16D).

In all anti HER2 mAbs that were tested, the combined screening of the diverse and focused libraries correctly identified the linear epitope of HER2, which corresponded to the published immunogen sequence used for raising the anti-HER2 mAbs.

### Example 6: Identification of Structural HER2 Epitopes

To demonstrate that the systems and methods provided can identify structural epitopes for the anti-HER2 mAbs, binding of Trastuzumab Fab monoclonal antibody (Herceptin) to a diverse and focused library was performed to identify the three linear components that constitute the structural epitope recognized by Herceptin.

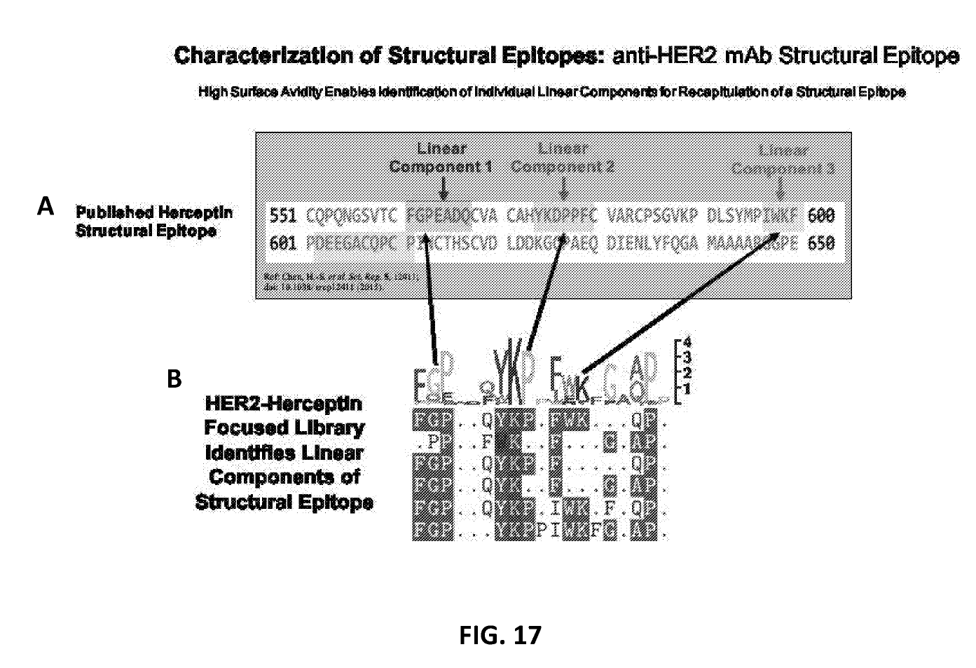

First, binding of Herceptin to a diverse library as described in Examples 1 and 2 was performed to identify enriched motifs in peptides bound by Herceptin to predict the linear components of the structural epitope. The three individual linear components of the HER2 structural epitope (FIG. 17A) recognized by Trastuzumab Fab (Herceptin): FGPEADQ, KDPPFC, and IWKFPDEEGACQPC (Chen, H.-S. et al. Sci. Rep. 5, 12411; doi: 10.1038/srep12411 [2015]). The motifs enriched in the significant peptides were subsequently used to identify 3 input regions of the HER2 target protein. A focused library was designed based on an input sequence that comprised the three motifs corresponding to the three structural components appended to each other. The focused library was screened using Herceptin, and the identified significant peptides were aligned to identify the conserved amino acids and their positions relative to the sequence of the published structural epitope.

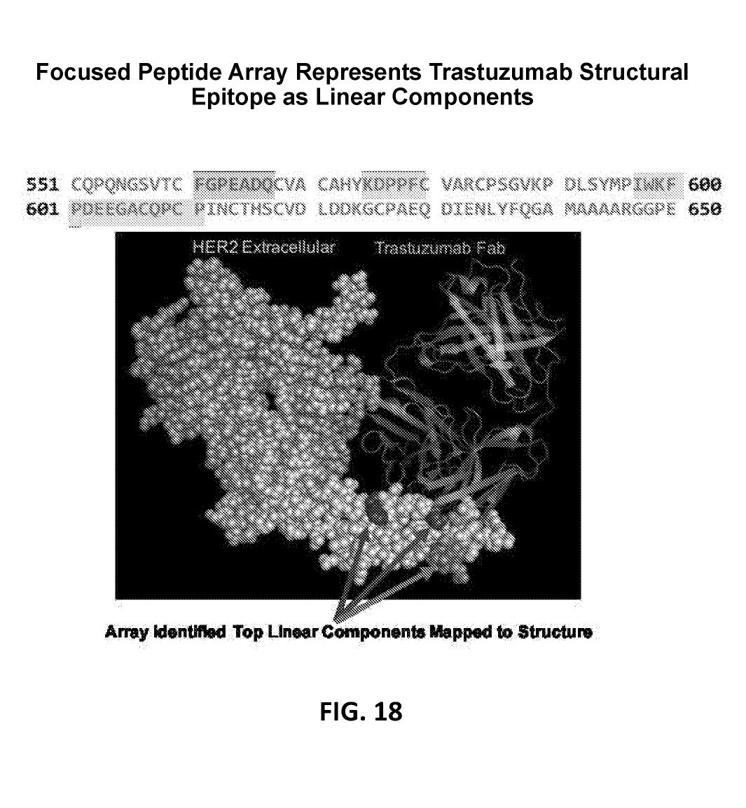

An exemplary alignment of the top significant peptides identified from the focused library is shown in FIG. 17B. The positions of each of the linear components were mapped to the sequence comprising the component of the structural epitope (FIG. 17A). FIG. 18 shows the crystal structure of Trastuzamb (BLASTP Ident Score at https://www.ncbi.nlm.nih.gov/books/NBK62051/), and its positioning relative to the extracellular portion of HER2. The colored portions of HER2 represent the individual linear components identified from peptide sequences in the focused library.

The data show that the entire sequence of a structural epitope was identified. These findings further corroborate the capability of the method provided to recapitulate the biological binding interactions between HER2 mAbs and its HER2 target.

### Example 7: Identification of Unknown Antibody Targets from Entire Proteome

Three mAbs Cell Signaling 2165 (Clone 29D8), Thermo MA5-13675 (Clone 3B5), and Santa Cruz SC-33684 (Clone 3B5) were used to demonstrate the capability of the systems and methods provided to identify unknown protein targets from an entire proteome.

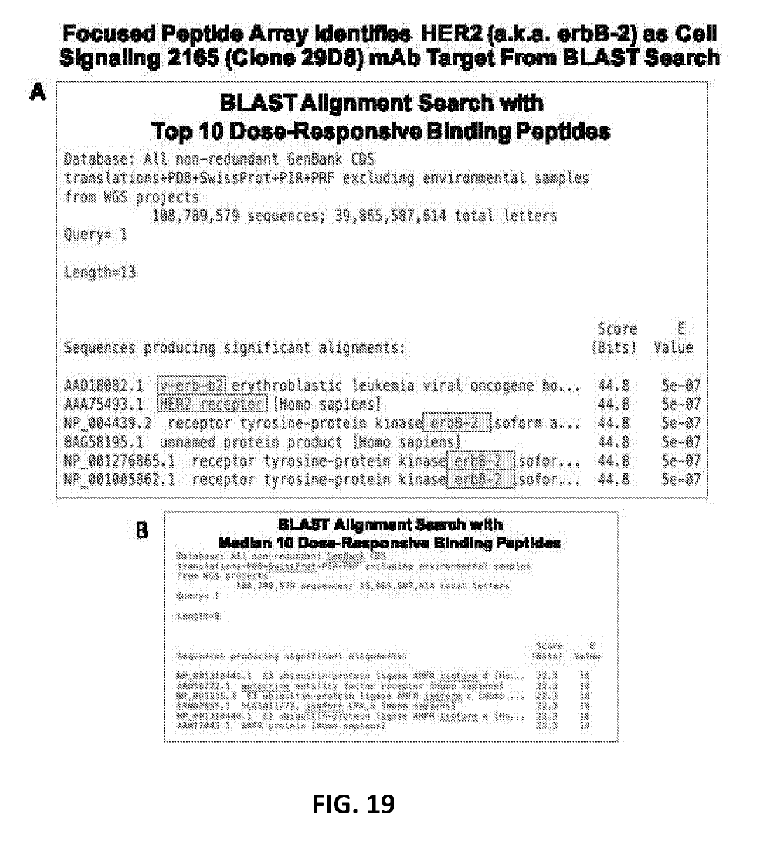

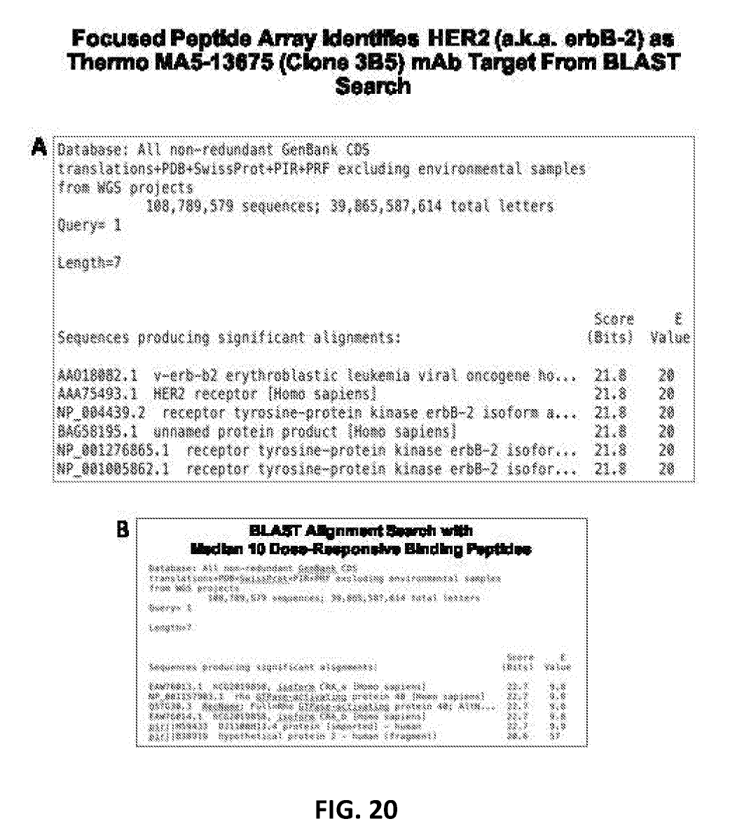

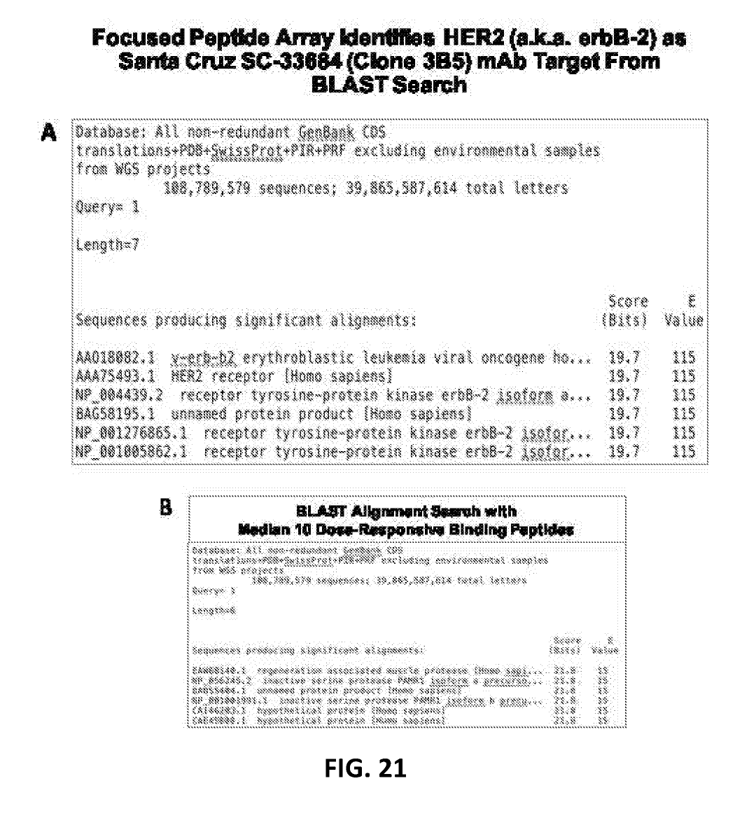

Individual peptides, including significant peptides, and enriched motifs for each of the mAbs were first identified from the corresponding diverse libraries. A query of a proteome for the presence of these short motifs would typically result in many alignments, most of which would be to sequences unrelated to the true target being sought. Subsequent design of sequences comprising the 3-4mer motifs, and screening of the resultant focused library identified 9-12 mer sequences for which the exact matches in the human proteome were found. FIGS. 19A, 20A, and 21A show the results of BLAST alignments of epitope sequences identified when queried using the top 10 individual peptides, in this case significant peptides, identified from screening the focused corresponding focused libraries. These figures show that all the highest scoring alignments were to HER2 protein, which is also known as v-erb-b2. In contrast, significant peptides having median specificity scores did not identify the relevant HER2 sequence when aligned to the human proteome (FIG. 19B, 20B, 21C).

These data show that unknown, target proteins for antibodies can be identified with high reliability, as shown by the BLAST score.

### Example 8: Determining the Specificity of an Anti-HER2 mAb

The specificity of monoclonal antibodies can be determined using the diverse and focused libraries described above.

First, the binding specificity of the peptides on both the diverse and focused libraries can be determined as described in Example 2. Having identified dose-responsive individual peptides, including significant peptides, from the focused library, the degree of conservation of the amino acids can be used to determine the specificity of the mAb. In one instance, the sum of the bits for all conserved amino acids that identify the consensus sequence of the epitope, e.g., FIG. 14D, can be compared to the sum of the bits obtained for the same putative epitope sequence when using a reference antibody or a panel of reference antibodies known to be unrelated to the mAb that was used to identify the true epitope. For example, a panel of 10 mAbs unrelated to anti-HER2 antibodies can be used as a mixture for binding to the diverse and focused libraries to provide individual peptides, including significant peptides that, when aligned would provide a bits score for the amino acids that may be conserved across the individual peptides.

Thus, the specificity of an antibody for an epitope can be defined by the degree of amino acid conservation in the putative epitope sequence.

### Example 9. Method for Determining the Propensity of an Antibody for a Set of Different Proteins

Binding of the mAb (Cell Signalling (#2165)) to HER2 and to EGFR was performed to demonstrate that the diverse and focused peptide array libraries provided can be used to determine the propensity of an antibody for binding to different protein targets. An algorithm was developed.

A first set of individual peptides, including significant peptides, was determined from the binding of the anti-HER2 mAb to a peptide array library, and a second set of individual peptides, including significant peptide, was determined from the anti-EGFR mAb to a same peptide array library. Enriched individual peptides, including significant peptide, motifs were identified for each of the two sets, and the enriched motifs were aligned to the corresponding target protein. The alignments of the motifs for each set were performed using 3 levels of alignment stringency:

High stringency (exact alignment)

Moderate-stringency (allows for small gaps and for amino acid substitutions), and

Low-Stringency (allows for wider gaps and for amino acid substitutions).

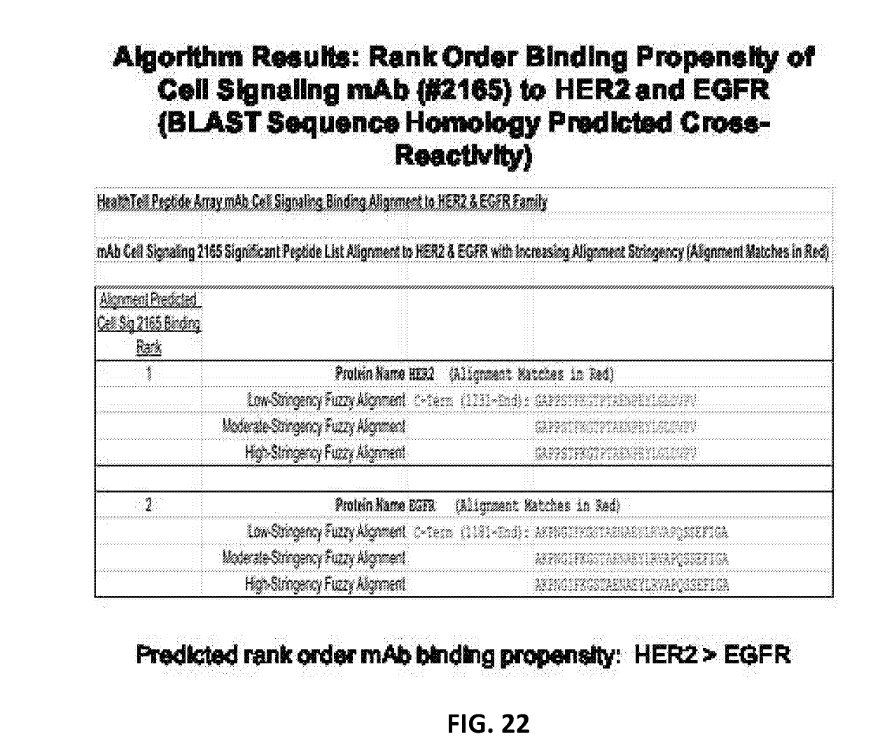

Each alignment identified specific residues in each target. In both cases, the stricter the rules of alignment i.e. with increasing alignment stringency, the stricter were the resulting alignments, as shown by the residues marked in red (FIG. 22). Each alignment can be scored by the number of “red” residues.

FIG. 22 shows that under the same alignment stringency rules, the mAb was predicted to bind EGFR to a lesser degree relative to the binding to HER2, i.e. the mAb has a greater propensity for binding to HER2 than to EGFR.

The propensity of an antibody for binding to a target protein can be determined using enriched motifs identified from the individual peptides (in this case significant peptides) of a diverse library, and/or from the individual peptides (in this case significant peptides) of a focused library.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of in situ synthesizing a chemical library on a substrate, the chemical library comprising a plurality of molecules, the method comprising:
(a) receiving a biological sequence and a number of synthesis steps;
(b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask;
(c) assigning at least one monomer to each patterned mask; and
(d) coupling the monomers onto the features to form molecules;
wherein (c) and (d) assembles one said synthesis step and the synthesis step is repeated.

2. The method of claim 1, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

3. The method of claim 1, wherein the input biological sequence comprises a disease-related epitope.

4. The method of claim 1, wherein the input biological sequence comprises a peptide sequence.

5. The method of claim 1, wherein the input biological sequence comprises an epitope sequence.

6. The method of claim 1, wherein the input biological sequence comprises a random sequence.

7. The method of claim 1, further comprising deriving an ordered list of monomers from the input biological sequence.

8. The method of claim 7, wherein a size of the ordered list is the number of the synthesis steps.

9. The method of claim 7, wherein the ordered list of monomers comprises the input biological sequence.

10. The method of claim 9, wherein the ordered list of monomers comprises the input biological sequence in a reversed order.

11. The method of claim 7, wherein the molecules are peptides or nucleic acids.

12. The method of claim 7, wherein the ordered list of monomers comprises a sequence of amino acids.

13. The method of claim 7, wherein the ordered list of monomers comprises a sequence of nucleotides.

14. The method of claim 1, wherein a number of the plurality of the patterned masks is less than 10, 15, 20, 25, 30, 35, 40, 45, 50, 60, 70, 80, 90, or 100.

15. The method of claim 1, wherein a number of the plurality of the patterned masks is the number of the synthesis steps.

16. The method of claim 1, wherein about 20% to about 50% of the activated designation features in each sequential patterned mask overlaps with the activated designation features of an immediately preceding patterned mask.

17. The method of claim 1, wherein about 30% to about 45% of the activated designation features in each sequential patterned mask overlaps with the activated designation features of an immediately preceding patterned mask.

18. The method of claim 1, wherein the synthesis step is based on photolithography.

19. The method of claim 1, wherein a feature on the substrate is about 0.5 micron to about 200 microns in diameter and a center-to-center distance of about 1 micron to about 300 microns on center.

20. The method of claim 1, wherein at least 40% of the molecules in the library are distinct.

21. The method of claim 1, wherein at least 50% of the molecules in the library are distinct.

22. The method of claim 1, wherein at least 60% of the molecules in the library are distinct.

23. The method of claim 1, wherein at least 70% of the molecules in the library are distinct.

24. The method of claim 1, wherein at least 80% of molecules in the library are distinct.

25. The method of claim 1, wherein at least 90% of molecules in the library are distinct.

26. The method of claim 1, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

27. The method of claim 1, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length.

28. The method of claim 1, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

29. The method of claim 1, wherein the library comprises a median monomer length equal to a length of the biological sequence.

30. The method of claim 1, wherein the library comprises a median monomer length longer than 40%, 50%, 60%, 70%, 80%, or 90% of a length of the biological sequence.

31. The method of claim 1, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

32. The method of claim 1, wherein the substrate is selected from the group consisting of arrays, wafers, slides and beads.

33. The method of claim 1, wherein the synthesized chemical library comprises peptides, nucleotides or a combination thereof.

34. The method of claim 33, wherein the peptides are about 5 to about 25 amino acids in length.

35. The method of claim 34, wherein the amino acids C, I, and M, and optionally Q and E, are not included in the amino acids available for peptide synthesis.

36. The method of claim 1, wherein the chemical library is synthesized with a surface spacer capable of cyclizing under oxidizing conditions.

37. The method of claim 36, wherein the surface spacer is Cys-Gly-Pro-Gly-Xaaₙ-Gly-Pro-Gly-Cys or Cys-(PEG3)-Xaaₙ-(PEG3)-Cys.

38. The method of claim 1, wherein the chemical library is synthesized with a surface spacer capable of cyclizing with an ester linkage.

39. The method of claim 38, wherein the ester linkage is a homobifunctional di-NHS ester linkage.

40. The method of claim 38, wherein the surface spacer is Lys-(PEG3)-Xaaₙ-(PEG3)-Lysine.

41. The method of claim 1, wherein the substrate is coated with a hydrophilic monolayer.

42. The method of claim 41, wherein the hydrophilic monolayer comprises polyethylene glycol (PEG), polyvinyl alcohol, carboxymethyl dextran, and combinations thereof.

43. The method of claim 41, wherein the hydrophilic monolayer is homogeneous.

44. An in situ synthesized chemical library, the chemical library comprising a plurality of molecules, wherein the synthesis uses patterned steps to construct the library on a substrate, comprising:
(a) receiving a biological sequence and a number of synthesis steps;
(b) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask;
(c) assigning at least one monomer to each patterned mask; and
(d) coupling the monomers onto the features to form molecules;
wherein (c) and (d) assembles one said synthesis step and the synthesis step is repeated.

45. The library of claim 44, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

46. The library of claim 44, wherein the input biological sequence comprises a disease-related epitope.

47. The library of claim 44, wherein the input biological sequence comprises a peptide sequence.

48. The library of claim 44, wherein the input biological sequence comprises an epitope sequence.

49. The library of claim 44, wherein the input biological sequence comprises a random sequence.

50. The library of claim 44, further comprising deriving an ordered list of monomers from the input biological sequence.

51. The library of claim 50, wherein a size of the ordered list is the number of the synthesis steps.

52. The library of claim 50, wherein the ordered list of monomers comprises the input biological sequence.

53. The library of claim 50, wherein the ordered list of monomers comprises the input biological sequence in a reversed order.

54. The library of claim 44, wherein the molecules comprise peptides or nucleic acids.

55. The library of claim 50, wherein the ordered list of monomers comprises a sequence of amino acids.

56. The library of claim 50, wherein the ordered list of monomers comprises a sequence of nucleotides.

57. The library of claim 44, wherein a number of the plurality of the patterned masks is less than 10, 15, 20, 25, 30, 35, 40, 45, 50, 60, 70, 80, 90, or 100.

58. The library of claim 44, wherein a number of the plurality of the patterned masks is the number of the synthesis steps.

59. The library of claim 44, wherein about 20% to about 50% of the activated designation features in each sequential patterned mask overlaps with the activated designation features of an immediately preceding patterned mask.

60. The library of claim 44, wherein about 30% to about 45% of the activated designation features in each sequential patterned mask overlaps with the activated designation features of an immediately preceding patterned mask.

61. The library of claim 44, wherein the synthesis step is based on photolithography.

62. The library of claim 44, wherein a feature on the substrate is about 0.5 micron to about 200 microns in diameter and a center-to-center distance of about 1 micron to about 300 microns on center.

63. The library of claim 44, wherein at least 40% of the molecules in the library are distinct.

64. The library of claim 44, wherein at least 50% of the molecules in the library are distinct.

65. The library of claim 44, wherein at least 60% of the molecules in the library are distinct.

66. The library of claim 44, wherein at least 70% of the molecules in the library are distinct.

67. The library of claim 44, wherein at least 80% of the molecules in the library are distinct.

68. The library of claim 44, wherein at least 90% of the molecules in the library are distinct.

69. The library of claim 44, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

70. The library of claim 44, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length.

71. The library of claim 44, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

72. The library of claim 44, wherein the library comprises a median monomer length equal to a length of the biological sequence.

73. The library of claim 44, wherein the library comprises a median monomer length longer than 40%, 50%, 60%, 70%, 80%, or 90% of a length of the biological sequence.

74. The library of claim 44, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

75. The library of claim 44, wherein the substrate is selected from the group consisting of arrays, wafers, slides and beads.

76. The library of claim 44, wherein the synthesized chemical library comprises peptides, nucleotides or a combination thereof.

77. The library of claim 76, wherein the peptides are about 5 to about 25 amino acids in length.

78. The library of claim 77, wherein the amino acids C, I, and M, and optionally Q and E, are not included in the amino acids available for peptide synthesis.

79. The library of claim 44, wherein the chemical library is synthesized with a surface spacer capable of cyclizing under oxidizing conditions.

80. The library of claim 79, wherein the surface spacer is Cys-Gly-Pro-Gly-Xaaₙ-Gly-Pro-Gly-Cys or Cys-(PEG3)-Xaaₙ-(PEG3)-Cys.

81. The library of claim 44, wherein the chemical library is synthesized with a surface spacer capable of cyclizing with an ester linkage.

82. The library of claim 81, wherein the ester linkage is a homobifunctional di-NHS ester linkage.

83. The library of claim 82, wherein the surface spacer is Lys-(PEG3)-Xaaₙ-(PEG3)-Lysine.

84. The library of claim 44, wherein the substrate is coated with a hydrophilic monolayer.

85. The library of claim 84, wherein the hydrophilic monolayer comprises polyethylene glycol (PEG), polyvinyl alcohol, carboxymethyl dextran, and combinations thereof.

86. The library of claim 84, wherein the hydrophilic monolayer is homogeneous.

87. A computing system for simulating in situ synthesis of a chemical library on a substrate, the chemical library comprising a plurality of molecules, comprising:
(a) a processor and a memory;
(b) a computer program including instructions executable by the processor, the computer program comprising:
(1) a receiving module configured to receive a biological sequence and a number of synthesis steps;
(2) a simulation module configured to: (i) determine a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately preceding patterned mask; (ii) assign at least one monomer to each patterned mask; and (iii) couple the monomers onto the features to form molecules;
wherein (i), (ii) and (iii) assembles one said synthesis step and the synthesis step is repeated.

88. The system of claim 87, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

89. The system of claim 87, wherein the input biological sequence comprises a disease-related epitope.

90. The system of claim 87, wherein the input biological sequence comprises a peptide sequence.

91. The system of claim 87, wherein the input biological sequence comprises an epitope sequence.

92. The system of claim 87, wherein the input biological sequence comprises a random sequence.

93. The system of claim 87, further comprising deriving an ordered list of monomers from the input biological sequence.

94. The system of claim 93, wherein a size of the ordered list is the number of the synthesis steps.

95. The system of claim 93, wherein the ordered list of monomers comprises the input biological sequence.

96. The system of claim 95, wherein the ordered list of monomers comprises the input biological sequence in a reversed order.

97. The system of claim 87, wherein the molecules comprises peptides or nucleic acids.

98. The system of claim 87, wherein the ordered list of monomers comprises a sequence of amino acids.

99. The system of claim 87, wherein the ordered list of monomers comprises a sequence of nucleotides.

100. The system of claim 87, wherein a number of the plurality of the patterned masks is less than 10, 15, 20, 25, 30, 35, 40, 45, 50, 60, 70, 80, 90, or 100.

101. The system of claim 87, wherein a number of the plurality of the patterned masks is the number of the synthesis steps.

102. The system of claim 87, wherein about 20% to about 50% of the activated designation features in each sequential patterned mask overlaps with the activated designation features of an immediately preceding patterned mask.

103. The system of claim 87, wherein about 30% to about 45% of the activated designation features in each sequential patterned mask overlaps with the activated designation features of an immediately preceding patterned mask.

104. The system of claim 87, wherein the synthesis step is based on photolithography.

105. The system of claim 87, wherein a feature on the substrate is about 0.5 micron to about 200 microns in diameter and a center-to-center distance of about 1 micron to about 300 microns on center.

106. The system of claim 87, wherein at least 40% of the molecules in the library are distinct.

107. The system of claim 87, wherein at least 50% of the molecules in the library are distinct.

108. The system of claim 87, wherein at least 60% of the molecules in the library are distinct.

109. The system of claim 87, wherein at least 70% of the molecules in the library are distinct.

110. The system of claim 87, wherein at least 80% of the molecules in the library are distinct.

111. The system of claim 87, wherein at least 90% of the molecules in the library are distinct.

112. The system of claim 87, wherein at least 50% of the molecules in the library are at least 3, 4, 5, 6, 7, 8, 9, 10, 11, 12, 13, 14, 15, 16, 17, 18, 19, 20, 30, 40, 50, 60, 70, 80, 90, or 100 monomers in length.

113. The system of claim 87, wherein at least 50% of the molecules in the library are at most 3, 4, 5, 6, 7, 8, 9, 10, 11, 12, 13, 14, 15, 16, 17, 18, 19, 20, 30, 40, 50, 60, 70, 80, 90, or 100 monomers in length.

114. The system of claim 87, wherein the molecules in the library comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

115. The system of claim 87, wherein the library comprises a median monomer length equal to a length of the biological sequence.

116. The system of claim 87, wherein the library comprises a median monomer length longer than 40%, 50%, 60%, 70%, 80%, or 90% of a length of the biological sequence.

117. The system of claim 87, wherein the library comprises a median monomer length shorter than 60%, 70%, 80%, 90%, 100%, 110%, 120%, 130%, 140%, 150%, 160%, 170%, 180%, 190%, or 200% of a length of the biological sequence.

118. The system of claim 87, wherein the substrate is selected from the group consisting of arrays, wafers, slides and beads.

119. The system of claim 87, wherein the synthesized chemical library comprises peptides, nucleotides or a combination thereof.

120. The system of claim 119, wherein the peptides are about 5 to about 25 amino acids in length.

121. The system of claim 120, wherein the amino acids C, I, and M, and optionally Q and E, are not included in the amino acids available for peptide synthesis.

122. The system of claim 87, wherein the chemical library is synthesized with a surface spacer capable of cyclizing under oxidizing conditions.

123. The system of claim 122, wherein the surface spacer is Cys-Gly-Pro-Gly-XaaₙGly-Pro-Gly-Cys or Cys-(PEG3)-Xaaₙ-(PEG3)-Cys.

124. The system of claim 87, wherein the chemical library is synthesized with a surface spacer capable of cyclizing with an ester linkage.

125. The system of claim 124, wherein the ester linkage is a homobifunctional di-NHS ester linkage.

126. The system of claim 125, wherein the surface spacer is Lys-(PEG3)-Xaaₙ-(PEG3)-Lysine.

127. The system of claim 87, wherein the substrate is coated with a hydrophilic monolayer.

128. The system of claim 127, wherein the hydrophilic monolayer comprises polyethylene glycol (PEG), polyvinyl alcohol, carboxymethyl dextran, and combinations thereof.

129. The system of claim 127, wherein the hydrophilic monolayer is homogeneous.

130. A method for in situ synthesizing a peptide array, the method comprising:
(a) receiving an input amino acid sequence;
(b) determining a number of synthesis steps;
(c) determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
(d) assigning at least one monomer to each patterned mask; and
(e) coupling the monomers onto the features, wherein (c) and (d) assembles one said synthesis step and said synthesis step is repeated to form the peptide array.

131. The method of claim 130, wherein the number of synthesis steps is larger than 50%, 60%, 70%, 80%, 90%, 100%, 110%, 120%, 130%, 140%, 150%, 160%, 170%, 180%, 190%, or 200% of a length of the biological sequence.

132. The method of claim 130, wherein the input sequence comprises a disease-related epitope.

133. The method of claim 130, wherein the input sequence comprises a peptide sequence.

134. The method of claim 130, wherein the input sequence comprises an epitope sequence.

135. The method of claim 130, further comprising deriving an ordered list of monomers from the input sequence.

136. The method of claim 135, wherein a size of the ordered list is the number of the synthesis steps.

137. The method of claim 135, wherein the ordered list of monomers comprises the input sequence.

138. The method of claim 137, wherein the ordered list of monomers comprises the input sequence in a reversed order.

139. The method of claim 135, wherein the ordered list of monomers comprises a sequence of amino acids.

140. The method of claim 130, wherein a number of the plurality of the patterned masks is less than 10, 15, 20, 25, 30, 35, 40, 45, 50, 60, 70, 80, 90, or 100.

141. The method of claim 130, wherein a number of the plurality of the patterned masks is the number of the synthesis steps.

142. The method of claim 130, wherein about 20% to about 50% of the activated designation features in each sequential patterned mask overlaps with the activated designation features of an immediately preceding patterned mask.

143. The method of claim 130, wherein about 30% to about 45% of the activated designation features in each sequential patterned mask overlaps with the activated designation features of an immediately preceding patterned mask.

144. The method of claim 130, wherein the synthesis step is based on photolithography.

145. The method of claim 130, wherein a feature on the substrate is about 0.5 micron to about 200 microns in diameter and a center-to-center distance of about 1 micron to about 300 microns on center.

146. The method of claim 130, wherein at least 40% of the peptides on the array are distinct.

147. The method of claim 130, wherein at least 50% of the peptides on the array are distinct.

148. The method of claim 130, wherein at least 60% of the peptides on the array are distinct.

149. The method of claim 130, wherein at least 70% of the peptides on the array are distinct.

150. The method of claim 130, wherein at least 80% of the peptides on the array are distinct.

151. The method of claim 130, wherein at least 90% of the peptides on the array are distinct.

152. The method of claim 130, wherein at least 50% of the peptides on the array are at least 3, 4, 5, 6, 7, 8, 9, 10, 11, 12, 13, 14, 15, 16, 17, 18, 19, 20, 30, 40, 50, 60, 70, 80, 90, or 100 monomers in length.

153. The method of claim 130, wherein at least 50% of the peptides on the array are at most 3, 4, 5, 6, 7, 8, 9, 10, 11, 12, 13, 14, 15, 16, 17, 18, 19, 20, 30, 40, 50, 60, 70, 80, 90, or 100 monomers in length.

154. The method of claim 130, wherein the peptides on the array comprises a median length of 3, 4, 5, 6, 7, 8, 9, 10, 11, 12, 13, 14, 15, 16, 17, 18, 19, 20, 30, 40, 50, 60, 70, 80, 90, or 100 monomers.

155. The method of claim 130, wherein the array comprises a median peptide length equal to a length of the input sequence.

156. The method of claim 130, wherein the array comprises a median peptide length longer than 40%, 50%, 60%, 70%, 80%, or 90% of a length of the input sequence.

157. The method of claim 130, wherein the array comprises a median peptide length shorter than 60%, 70%, 80%, 90%, 100%, 110%, 120%, 130%, 140%, 150%, 160%, 170%, 180%, 190%, or 200% of a length of the input sequence.

158. The method of claim 130, wherein the peptides are about 5 to about 25 amino acids in length.

159. The method of claim 130, wherein the amino acids C, I, and M, and optionally Q and E, are not included in the amino acids available for peptide synthesis.

160. The method of claim 130, wherein the peptide array is synthesized with a surface spacer capable of cyclizing under oxidizing conditions.

161. The method of claim 160, wherein the surface spacer is Cys-Gly-Pro-Gly-XaaₙGly-Pro-Gly-Cys or Cys-(PEG3)-Xaaₙ-(PEG3)-Cys.

162. The method of claim 130, wherein the peptide array is synthesized with a surface spacer capable of cyclizing with an ester linkage.

163. The method of claim 162, wherein the ester linkage is a homobifunctional di-NHS ester linkage.

164. The method of claim 160, wherein the surface spacer is Lys-(PEG3)-Xaaₙ-(PEG3)-Lysine.

165. The method of claim 130, wherein the peptide array is coated with a hydrophilic monolayer.

166. The method of claim 165, wherein the hydrophilic monolayer comprises polyethylene glycol (PEG), polyvinyl alcohol, carboxymethyl dextran, and combinations thereof.

167. The method of claim 165, wherein the hydrophilic monolayer is homogeneous.

168. An array comprising a plurality of in situ synthesized peptides on the array, the peptides produced by a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask.

169. A method for characterizing antibody binding against at least one protein target, the method comprising:
(a) contacting a peptide array with said antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides;
(b) aligning the individual peptides to said at least one protein target, wherein the alignments between the individual peptides of step (a) and at least one protein target are assigned alignment scores; and
(c) characterizing binding of the antibody against the at least one protein target using the alignment scores of step (b).

170. The method of claim 169, wherein the predetermined threshold is a binding signal in the presence of competitor peptides within at least 20-fold of the binding signal in the absence of competitor peptides.

171. The method of claim 169, wherein the predetermined threshold is a binding signal in the presence of competitor peptides of at least 5% of the binding signal as compared in the absence of competitor.

172. The method of claim 169, wherein the competitor peptides comprise a biological sample.

173. The method of claim 169, wherein the biological sample is serum.

174. The method of claim 169, wherein the competitor peptides are derived from the target protein.

175. The method of claim 174, wherein the competitor peptides are at least 50% similar to the target protein.

176. The method of claim 169, wherein the competitor peptides are derived from a known epitope of the antibody.

177. The method of claim 176, wherein the competitor peptides are at least 50% similar to the known epitope of the antibody.

178. The method of claim 169, wherein the competitor peptides comprise a biological sample and a peptide of any of claims 174 to 177.

179. The method of claim 169, wherein the peptide array comprises at least 1000 unique peptides.

180. The method of claim 169, wherein the peptide array comprises at least 10,000 unique peptides.

181. The method of claim 169, wherein the peptide array comprises at least 100,000 unique peptides.

182. The method of claim 169, wherein the peptide array comprises at least 1,000,000 unique peptides.

183. The method of claim 169, wherein the peptide array is in situ synthesized.

184. The method of claim 183, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

185. The method of claim 169, wherein the binding signal is measured as an intensity of the signal in the absence and presence of the competitor peptides at one or more concentrations.

186. The method of claim 169, wherein an apparent Kd is obtained in the presence and absence of the competitor peptides at one or more concentrations.

187. The method of claim 169, wherein at least one additional antibody is contacted with the peptide array, and the alignment scores obtained with each antibody are ranked to determine the propensity of each antibody to bind to the protein target.

188. The method of claim 169, further comprising determining a metric score for each antibody, wherein each antibody is assigned a single binding profile metric derived from the combination of the alignment scores from step (b) in claim 169 and the signal of the individual peptides of step (a) with more than one aligned position from step (b).

189. The method of claim 169, further comprising determining a metric score for each antibody, wherein each antibody is assigned a single specificity profile metric derived from the combination of the alignment scores from step (b) in claim 169, the number of peptides with more than one aligned position from step (b) and the signal of the individual peptides of step (a) with more than one aligned position from step (b).

190. A method for identifying an antibody epitope in a target protein, the method comprising:
(a) contacting a peptide array with said antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the individual peptides to said at least one protein target, wherein the alignments between the individual peptides of step (a) and at least one protein target are assigned alignment scores; and
(c) determining conserved amino acids in the individual peptides of step (a) to identify a conserved binding peptide motif and aligning the individual motifs to said at least one target protein in order to identify at least one antibody epitope of the target protein.

191. The method of claim 190, wherein the predetermined threshold is a binding signal in the presence of competitor peptides within at least 20-fold of the binding signal in the absence of competitor peptides.

192. The method of claim 190, wherein the predetermined threshold is a binding signal in the presence of competitor peptides wherein the predetermined threshold is a binding signal in the presence of competitor peptides of at least 5% of the binding signal as compared in the absence of competitor.

193. The method of claim 190, wherein the competitor peptides comprise a biological sample.

194. The method of claim 190, wherein the biological sample is serum.

195. The method of claim 190, wherein the competitor peptides are derived from the target protein.

196. The method of claim 195, wherein the competitor peptides are at least 50% similar to the target protein.

197. The method of claim 190, wherein the competitor peptides are derived from a known epitope of the antibody.

198. The method of claim 197, wherein the competitor peptides are at least 50% similar to the known epitope of the antibody.

199. The method of claim 190, wherein the competitor peptides comprise a biological sample and a peptide of any of claims 193 to 198.

200. The method of claim 190, wherein the peptide array comprises at least 1000 unique peptides.

201. The method of claim 190, wherein the peptide array comprises at least 10,000 unique peptides.

202. The method of claim 190, wherein the peptide array comprises at least 100,000 unique peptides.

203. The method of claim 190, wherein the peptide array comprises at least 1,000,000 unique peptides.

204. The method of claim 190, wherein the peptide array is in situ synthesized.

205. The method of claim 204, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

206. The method of claim 190, wherein the binding signal is measured as an intensity of the signal in the absence and presence of the competitor peptides at one or more concentrations.

207. The method of claim 190, wherein an apparent Kd is obtained in the presence and absence of the competitor peptides at one or more concentrations.

208. The method of claim 190, wherein at least one additional antibody is contacted with the peptide array, and the alignment scores obtained with each antibody are ranked to determine the propensity of each antibody to bind to the protein target.

209. The method of claim 190, further comprising determining a metric score for each antibody, wherein each antibody is assigned a single binding profile metric derived from the combination of the alignment scores from step (b) in claim 190 and the signal of the individual peptides of step (a) with more than one aligned position from step (b).

210. The method of claim 190, further comprising determining a metric score for each antibody, wherein each antibody is assigned a single specificity profile metric derived from the combination of the alignment scores from step (b) in claim 190, the number of peptides with more than one aligned position from step (b) and the signal of the individual peptides of step (a) with more than one aligned position from step (b).

211. The method of claim 190, further comprising aligning the at least one antibody epitope as a search criteria against a protein database.

212. The method of claim 211, wherein the protein database is a proteome database and wherein additional antibody target proteins and/or cross-reactive proteins are identified.

213. A method for characterizing antibody binding regions in a target protein, the method comprising:
(a) contacting a first peptide array with said antibody in the presence and absence of a plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides;
(b) creating a second peptide array using an input peptide sequence chosen from at least one of the individual peptides in step (a), a conserved motif derived from an alignment of the individuals peptides in step (a) or an aligned motif derived from an alignment of the individual peptides in step (a), the second peptide array synthesized by:
i. determining a number of synthesis steps;
ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (ii) and (iii) assembles one said synthesis step and said synthesis step is repeated to form the peptide array;
(c) contacting said second peptide array with said antibody to identify a second set of peptides; and
(d) contacting said second peptide array with said antibody in the presence of a plurality of competitor peptides, and identifying a second set of individual peptides from step (c) that exhibit a binding signal within a second predetermined threshold of the binding signal in step (c); and
(e) aligning said second set of individual peptides to said target protein and identifying regions in the target protein which align to the second set of individual peptides identified, thereby characterizing antibody binding regions in the target protein.

214. The method of claim 213, wherein the competitor peptides comprise a biological sample.

215. The method of claim 213, wherein the biological sample is serum.

216. The method of claim 213, wherein the competitor peptides are derived from the target protein.

217. The method of claim 216, wherein the competitor peptides are at least 50% similar to the target protein.

218. The method of claim 213, wherein the competitor peptides are derived from a known epitope of the antibody.

219. The method of claim 218, wherein the competitor peptides are at least 50% similar to the known epitope of the antibody.

220. The method of claim 213, wherein the competitor peptides comprise a biological sample and a peptide of any of claims 216 to 219.

221. The method of claim 213, wherein the peptide array comprises at least 1000 unique peptides.

222. The method of claim 213, wherein the peptide array comprises at least 10,000 unique peptides.

223. The method of claim 213, wherein the peptide array comprises at least 100,000 unique peptides.

224. The method of claim 213, wherein the peptide array comprises at least 1,000,000 unique peptides.

225. The method of claim 213, wherein the peptide array is in situ synthesized.

226. The method of claim 213, wherein the first peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

227. The method of claim 213, wherein the binding signal is measured as an intensity of the signal in the absence and presence of the competitor peptides at one or more concentrations.

228. The method of claim 213, wherein an apparent Kd is obtained in the presence and absence of the competitor peptides at one or more concentrations.

229. The method of claim 213, wherein at least one additional antibody is contacted with the peptide array, and the alignment scores obtained with each antibody are ranked to determine the propensity of each antibody to bind to the protein target.

230. The method of claim 213, further comprising determining a metric score for each antibody, wherein each antibody is assigned a single binding profile metric derived from the combination of the alignment scores from step (b) in claim 213 and the signal of the individual peptides of step (a) with more than one aligned position from step (b).

231. The method of claim 213, further comprising determining a metric score for each antibody, wherein each antibody is assigned a single specificity profile metric derived from the combination of the alignment scores from step (b) in claim 213, the number of peptides with more than one aligned position from step (b) and the signal of the individual peptides of step (a) with more than one aligned position from step (b).

232. The method of claim 213, further comprising aligning the at least one antibody epitope as a search criteria against a protein database.

233. The method of claim 232, wherein the protein database is a proteome database and wherein additional antibody target proteins and/or cross-reactive proteins are identified.

234. The method of claim 213, wherein the first predetermined threshold is a binding signal in the presence of competitor peptides within at least 20-fold of the binding signal in the absence of competitor peptides.

235. The method of claim 213, wherein the second predetermined threshold is a binding signal in the presence of competitor peptides within at least 20-fold of the binding signal in the absence of competitor peptides.

236. The method of claim 213, wherein the first predetermined threshold is a binding signal in the presence of competitor peptides of at least 5% of the binding signal as compared in the absence of competitor.

237. The method of claim 213, wherein the second predetermined threshold is a binding signal in the presence of competitor peptides of at least 5% of the binding signal as compared in the absence of competitor.

238. The method of claim 213, wherein the antibody binding region(s) is a linear epitope of the target protein.

239. The method of claim 213, wherein the antibody binding regions(s) is a structural epitope of the target region.

240. The method of claim 239, wherein steps (b) through din claim 213 are repeated with additional peptides chosen from the at least one of the individual peptides in step (a) of claim 213.

241. A method for identifying a target protein of an antibody, the method comprising:
(a) contacting a first peptide array with said antibody at one or more concentrations in the presence and absence of a plurality of competitor peptides at one or more concentrations to obtain one or more input amino acid sequences, wherein the identified input amino acid sequences exhibit a binding signal in the presence of the plurality of competitor peptides within a first predetermined threshold of the binding signal in the absence of the plurality of competitor peptides;
(b) obtaining one or more secondary peptide array(s) using one or more input amino acid sequences chosen from at least one of the individual peptides in step (a), a conserved motif derived from an alignment of the individuals peptides in step (a) or an aligned motif derived from an alignment of the individual peptides in step (a), the one or more secondary peptide arrays synthesized by:
i. determining a number of synthesis steps;
ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (ii) and (iii) assembles one said synthesis step and said synthesis step is repeated to form the peptide array;
(c) contacting each of said secondary peptide array(s) with said antibody in the presence and absence of the plurality of competitor peptides to obtain a set of peptide sequences, wherein the identified set of peptide sequences exhibit a binding signal measured in the presence of the plurality of competitor peptides within a second predetermined threshold of the binding signal measured in the absence of the plurality of competitor peptides;
(d) aligning said set of peptide sequences with each other to obtain at least one predictive binding motif; and
(e) aligning said predictive binding motif as a search criteria against a protein database, thereby identifying target proteins of the antibody based on the protein database search results score.

242. The method of claim 241, wherein the competitor peptides comprise a biological sample.

243. The method of claim 241, wherein the biological sample is serum.

244. The method of claim 241, wherein the competitor peptides are derived from the target protein.

245. The method of claim 244, wherein the competitor peptides are at least 50% similar to the target protein.

246. The method of claim 241, wherein the competitor peptides are derived from a known epitope of the antibody.

247. The method of claim 246, wherein the competitor peptides are at least 50% similar to the known epitope of the antibody.

248. The method of claim 241, wherein the competitor peptides comprise a biological sample and a peptide of any of claims 244 to 247.

249. The method of claim 241, wherein the peptide array comprises at least 1000 unique peptides.

250. The method of claim 241, wherein the peptide array comprises at least 10,000 unique peptides.

251. The method of claim 241, wherein the peptide array comprises at least 100,000 unique peptides.

252. The method of claim 241, wherein the peptide array comprises at least 1,000,000 unique peptides.

253. The method of claim 241, wherein the peptide array is in situ synthesized.

254. The method of claim 241, wherein the first peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

255. The method of claim 241, wherein the binding signal is measured as an intensity of the signal in the absence and presence of the competitor peptides at one or more concentrations.

256. The method of claim 241, wherein an apparent Kd is obtained in the presence and absence of the competitor peptides at one or more concentrations.

257. The method of claim 241, wherein at least one additional antibody is contacted with the peptide array, and the alignment scores obtained with each antibody are ranked to determine the propensity of each antibody to bind to the protein target.

258. The method of claim 241, further comprising determining a metric score for each antibody, wherein each antibody is assigned a single binding profile metric derived from the combination of the alignment scores from step (b) in claim 241 and the signal of the individual peptides of step (a) with more than one aligned position from step (b).

259. The method of claim 241, further comprising determining a metric score for each antibody, wherein each antibody is assigned a single specificity profile metric derived from the combination of the alignment scores from step (b) in claim 241, the number of peptides with more than one aligned position from step (b) and the signal of the individual peptides of step (a) with more than one aligned position from step (b).

260. The method of claim 241, further comprising aligning the at least one antibody epitope as a search criteria against a protein database.

261. The method of claim 260, wherein the protein database is a proteome database and wherein additional antibody target proteins and/or cross-reactive proteins are identified.

262. The method of claim 241, wherein the first predetermined threshold is a binding signal in the presence of competitor peptides within at least 20-fold of the binding signal in the absence of competitor peptides.

263. The method of claim 241, wherein the predetermined threshold is a binding signal in the presence of competitor peptides of at least 5% of the binding signal as compared in the absence of competitor.

264. A method for determining the propensity of antibody binding to at least one protein target, the method comprising:
(a) contacting a peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the individual peptides of step (a) to a first protein target, wherein the alignments between the individual peptides of step (a) and the first protein target are assigned alignment scores;
(c) repeating the alignment of individual peptides of step (a) with at least one additional protein target(s), wherein the alignments between the individual peptides of step (a) and the additional protein targets are assigned alignment scores; and
(d) comparing the alignment scores from steps (b) and (c) to obtain a relative propensity of the antibody to bind to said protein targets.

265. The method of claim 264, wherein the competitor peptides comprise a biological sample.

266. The method of claim 265, wherein the biological sample is serum.

267. The method of claim 264, wherein the competitor peptides are derived from the target protein.

268. The method of claim 264, wherein the competitor peptides are at least 50% similar to the target protein.

269. The method of claim 264, wherein the competitor peptides are derived from a known epitope of the antibody.

270. The method of claim 269, wherein the competitor peptides are at least 50% similar to the known epitope of the antibody.

271. The method of claim 264, wherein the competitor peptides comprise a biological sample and a peptide of any of claims 267 to 270.

272. The method of claim 264, wherein the peptide array comprises at least 1000 unique peptides.

273. The method of claim 264, wherein the peptide array comprises at least 10,000 unique peptides.

274. The method of claim 264, wherein the peptide array comprises at least 100,000 unique peptides.

275. The method of claim 264, wherein the peptide array comprises at least 1,000,000 unique peptides.

276. The method of claim 264, wherein the peptide array is in situ synthesized.

277. The method of claim 264, wherein the peptide array is synthesized by:
i. determining a number of synthesis steps;
ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (b) and (c) assembles one said synthesis step and said synthesis step is repeated to form the peptide array.

278. The method of claim 264, wherein the binding signal is measured as an intensity of the signal in the absence and presence of the competitor peptides at one or more concentrations.

279. The method of claim 264, wherein an apparent Kd is obtained in the presence and absence of the competitor peptides at one or more concentrations.

280. The method of claim 264, wherein at least one additional antibody is contacted with the peptide array, and the alignment scores obtained with each antibody are ranked to determine the propensity of each antibody to bind to the protein target.

281. The method of claim 264, further comprising determining a metric score for each antibody, wherein each antibody is assigned a single binding profile metric derived from the combination of the alignment scores from step (b) in claim 264 and the signal of the individual peptides of step (a) with more than one aligned position from step (b).

282. The method of claim 264, further comprising determining a metric score for each antibody, wherein each antibody is assigned a single specificity profile metric derived from the combination of the alignment scores from step (b) in claim 264, the number of peptides with more than one aligned position from step (b) and the signal of the individual peptides of step (a) with more than one aligned position from step (b).

283. The method of claim 264, further comprising aligning the at least one antibody epitope as a search criteria against a protein database.

284. The method of claim 283, wherein the protein database is a proteome database and wherein additional antibody target proteins and/or cross-reactive proteins are identified.

285. The method of claim 264, wherein the predetermined threshold is a binding signal in the presence of competitor peptides within at least 20-fold of the binding signal in the absence of competitor peptides.

286. The method of claim 264, wherein the predetermined threshold is a binding signal in the presence of competitor peptides of at least 5% of the binding signal as compared in the absence of competitor.

287. A method for determining the propensity of antibody binding to at least one protein target, the method comprising:
(a) contacting a first peptide array with an antibody at one or more concentrations in the presence and absence of a plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(b) aligning the one or more individual peptides of step (a) to obtain at least one predictive target motif;
(c) aligning the at least one predictive target motif to a first protein target, wherein the alignments between the individual peptides of step (a) and the first protein target are assigned alignment scores;
(d) repeating the alignment of at least one predictive target motif of step (b) with at least one additional protein target(s), wherein the alignments between the at least one predictive target motif of step (b) and the additional protein target(s) are assigned alignment scores; and
(e) comparing the alignment scores from steps (c) and (d) to obtain a relative propensity of the antibody to bind to said protein targets.

288. The method of claim 287, wherein the competitor peptides comprise a biological sample.

289. The method of claim 288, wherein the biological sample is serum.

290. The method of claim 287, wherein the competitor peptides are derived from the target protein.

291. The method of claim 290, wherein the competitor peptides are at least 50% similar to the target protein.

292. The method of claim 287, wherein the competitor peptides are derived from a known epitope of the antibody.

293. The method of claim 292, wherein the competitor peptides are at least 50% similar to the known epitope of the antibody.

294. The method of claim 287, wherein the competitor peptides comprise a biological sample and a peptide of any of claims 290 to 293.

295. The method of claim 287, wherein the peptide array comprises at least 1000 unique peptides.

296. The method of claim 287, wherein the peptide array comprises at least 10,000 unique peptides.

297. The method of claim 287, wherein the peptide array comprises at least 100,000 unique peptides.

298. The method of claim 287, wherein the peptide array comprises at least 1,000,000 unique peptides.

299. The method of claim 287, wherein the peptide array is in situ synthesized.

300. The method of claim 287, wherein the peptide array is synthesized by:
i. receiving an input amino acid sequence;
ii. determining a number of synthesis steps;
iii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iv. assigning at least one monomer to each patterned mask; and
v. coupling the monomers onto the features, wherein (c) and (d) assembles one said synthesis step and said synthesis step is repeated to form the peptide array.

301. The method of claim 287, wherein the binding signal is measured as an intensity of the signal in the absence and presence of the competitor peptides at one or more concentrations.

302. The method of claim 287, wherein an apparent Kd is obtained in the presence and absence of the competitor peptides at one or more concentrations.

303. The method of claim 287, wherein at least one additional antibody is contacted with the peptide array, and the alignment scores obtained with each antibody are ranked to determine the propensity of each antibody to bind to the protein target.

304. The method of claim 287, further comprising determining a metric score for each antibody, wherein each antibody is assigned a single binding profile metric derived from the combination of the alignment scores from step (b) in claim 287 and the signal of the individual peptides of step (a) with more than one aligned position from step (b).

305. The method of claim 287, further comprising determining a metric score for each antibody, wherein each antibody is assigned a single specificity profile metric derived from the combination of the alignment scores from step (b) in claim 287, the number of peptides with more than one aligned position from step (b) and the signal of the individual peptides of step (a) with more than one aligned position from step (b).

306. The method of claim 287, further comprising aligning the at least one antibody epitope as a search criteria against a protein database.

307. The method of claim 306, wherein the protein database is a proteome database and wherein additional antibody target proteins and/or cross-reactive proteins are identified.

308. The method of claim 287, wherein the predetermined threshold is a binding signal in the presence of competitor peptides within at least 20-fold of the binding signal in the absence of competitor peptides.

309. The method of claim 287, wherein the predetermined threshold is a binding signal in the presence of competitor peptides of at least 5% of the binding signal as compared in the absence of competitor.

310. A kit for characterizing antibody binding against at least one protein target, the kit comprising:
(a) providing a peptide array;
(b) providing a plurality of competitor peptides;
(c) providing instructions for a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides at one or more concentrations within a predetermined threshold of the binding signal measured in the absence of the plurality of competitor peptides;
(d) providing instructions for the user to align the individual peptides to said at least one protein target, wherein the alignments between the individual peptides of step (c) and at least one protein target are assigned alignment scores; and
(e) providing instructions for the user to characterize binding of the antibody against the at least one protein target using the alignment scores of step (d).

311. A kit for identifying an antibody epitope in a target protein, the kit comprising:
(a) providing a peptide array;
(b) providing a plurality of competitor peptides;
(c) providing instructions for a user to contact the peptide array with said antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions for the user to align the individual peptides to said at least one protein target, wherein the alignments between the individual peptides of step (c) and at least one protein target are assigned alignment scores; and
(e) providing instructions for the user to determine conserved amino acids in the individual peptides of step (c) to identify a conserved binding peptide motif and aligning the individual motifs to said at least one target protein in order to identify at least one antibody epitope of the target protein.

312. A kit for characterizing antibody binding regions in a target protein, the kit comprising:
(a) providing a first peptide array;
(b) providing a plurality of competitor peptides;
(c) providing instructions for a user to contact a first peptide array with an antibody in the presence and absence of the plurality of competitor peptides to obtain one or more individual peptides, wherein the identified one or more individual peptides exhibit a binding signal measured in the presence of the plurality of competitor peptides within a first predetermined threshold of the binding signal measured in the absence of the plurality of competitor peptides;
(d) providing instructions for a user to create a second peptide array using an input peptide sequence chosen from at least one of the individual peptides in step (c), a conserved motif derived from an alignment of the individuals peptides in step (c) or an aligned motif derived from an alignment of the individual peptides in step (c), the second peptide array synthesized by:
i. determining a number of synthesis steps;
ii. determining a plurality of patterned masks, wherein each patterned mask is assigned an activated or inactivated designation to each feature on the substrate, and wherein about 1% to about 75% of the activated designation features in each sequential patterned mask overlaps with the activated designation features of an immediately patterned mask;
iii. assigning at least one monomer to each patterned mask; and
iv. coupling the monomers onto the features, wherein (ii) and (iii) assembles one said synthesis step and said synthesis step is repeated to form the peptide array;
(e) providing instructions for the user to contact the second peptide array with the antibody to identify a second set of peptides;
(f) providing instructions for the user to contact the second peptide array with said antibody in the presence of the plurality of competitor peptides, and identifying a second set of individual peptides from step (e) that exhibit a binding signal within a second predetermined threshold of the binding signal in step (e); and
(g) providing instructions for a user to align said second set of individual peptides to said target protein and identifying regions in the target protein which align to the second set of individual peptides identified, thereby characterizing antibody binding regions in the target protein.

313. A kit for determining the propensity of antibody binding to at least one protein target, the kit comprising:
(a) providing a peptide array;
(b) providing a plurality of competitor peptides;
(c) providing instructions to a user to contact the peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions to the user to align the individual peptides of step (c) to a first protein target, wherein the alignments between the individual peptides of step (c) and the first protein target are assigned alignment scores;
(e) providing instructions to the user to repeat the alignment of individual peptides of step (c) with at least one additional protein target(s), wherein the alignments between the individual peptides of step (c) and the additional protein targets are assigned alignment scores; and
(f) providing instructions to the user to compare the alignment scores from steps (c) and (d) to obtain a relative propensity of the antibody to bind to said protein targets.

314. A kit for determining the propensity of antibody binding to at least one protein target, the kit comprising:
(a) providing a first peptide array;
(b) providing a plurality of competitor peptides;
(c) providing instructions for a user to contact the first peptide array with an antibody at one or more concentrations in the presence and absence of the plurality of competitor peptides at one or more concentrations to obtain one or more individual peptides, wherein the identified one or more individual peptides exhibit a binding signal measured in the presence of the plurality of competitor peptides within a predetermined threshold of the binding signal measured in the absence of the plurality of competitor peptides;
(d) providing instructions for the user to align the one or more individual peptides of step (c) to obtain at least one predictive target motif;
(e) providing instructions for the user to align the at least one predictive target motif to a first protein target, wherein the alignments between the individual peptides of step (c) and the first protein target are assigned alignment scores;
(f) providing instructions for the user to repeat the alignment of at least one predictive target motif of step (e) with at least one additional protein target(s), wherein the alignments between the at least one predictive target motif of step (e) and the additional protein target(s) are assigned alignment scores; and
(g) providing instructions for the user to compare the alignment scores from steps (c) and (d) to obtain a relative propensity of the antibody to bind to said protein targets.
